(19) United States
(12) Patent Application Publication
(10) Pub. No.: US 2020/0138304 A1
(43) Pub. Date: May 7, 2020

(54) ANTENNA DEVICE FOR BIOLOGICAL MEASUREMENT, PULSE WAVE MEASURING DEVICE, BLOOD PRESSURE MEASURING DEVICE, APPARATUS, BIOLOGICAL INFORMATION MEASURING METHOD, PULSE WAVE MEASURING METHOD, AND BLOOD PRESSURE MEASURING METHOD

(71) Applicants: Omron Corporation, Kyoto (JP); OMRON HEALTHCARE Co., Ltd., Kyoto (JP)

(72) Inventors: Hisashi Ozawa, Kyoto (JP); Keisuke Saito, Osaka (JP); Keigo Kamada, Tokyo (JP); Yasuhiro Kawabata, Kyoto (JP)

(73) Assignees: Omron Corporation, Kyoto (JP); OMRON HEALTHCARE Co., Ltd., Kyoto (JP)

(21) Appl. No.: 16/735,074

(22) Filed: Jan. 6, 2020

Related U.S. Application Data
(63) Continuation of application No. PCT/JP2018/024034, filed on Jun. 25, 2018.

(30) Foreign Application Priority Data
Jul. 21, 2017 (JP) 2017-142221

(51) Int. Cl. A61B 5/021; H01Q 1/27; H04B 1/3827; H04B 17/336; A61B 5/00; A61B 5/022

(52) U.S. Cl. CPC A61B 5/02125; H01Q 1/273; H04B 1/385; A61B 5/0507; A61B 5/6802; A61B 5/02225; H04B 2001/3855; H04B 17/336

(57) ABSTRACT

An antenna device for biological measurement according to the present invention includes a belt to be worn as surrounding a measurement target site of a living body. A transmission/reception antenna group is provided to the belt and includes a plurality of antenna elements. In a wearing state where the belt is worn as surrounding an outer surface of the measurement target site, a radio wave is emitted toward the measurement target site using any one of the antenna elements as a transmission antenna. A reflected radio wave is received using any one of the antenna elements as a reception antenna. A transmission/reception antenna pair formed of the transmission antenna and the reception antenna is selected by switching or weighted among the plurality of antenna elements based on a reception output.

### CROSS-REFERENCE TO RELATED APPLICATION

This is a continuation application of International Application No. PCT/JP2018/024034, with an International filing date of Jun. 25, 2018, which claims priority of Japanese Patent Application No. 2017-142221 filed on Jul. 21, 2017, the entire content of which is hereby incorporated by reference.

### TECHNICAL FIELD

The present invention relates to an antenna device for biological measurement, and more particularly to an antenna device for biological measurement that emits radio waves toward a measurement target site of a living body or receives radio waves from the measurement target site to measure biological information. The present invention also relates to a pulse wave measuring device, a blood pressure measuring device, and an apparatus provided with such an antenna device for biological measurement. The present invention also relates to a biological information measuring method for emitting radio waves toward a measurement target site of a living body or receiving radio waves from the measurement target site. The present invention also relates to a pulse wave measuring method and a blood pressure measuring method, including such a biological information measuring method.

### BACKGROUND ART

Conventionally, as this type of antenna device for biological measurement, for example, as disclosed in Patent Document 1 (JP 5879407 B), there is a known device in which a transmission (emission) antenna and a reception antenna that face a measurement target site are provided and the radio wave (measurement signal) is emitted from the transmission antenna toward the measurement target site (target object), and the radio wave (reflection signal) reflected by the measurement target site is received by the reception antenna to measure biological information.

### SUMMARY OF INVENTION

By the way, when measuring a pulse wave (or a signal related to a pulse wave) as biological information for example, a wrist through which an artery passes may be used as a measurement target site. For example, there may be an aspect in which a belt (or cuff) of a wearable device to be worn around a wrist is provided with a transmission antenna and a reception antenna (which is referred to as “transmission/reception antenna pair” as appropriate) arranged spaced apart from each other in a width direction of the belt (corresponding to the longitudinal direction of the wrist) to measure a pulse wave signal using the transmission/reception antenna pair. In this aspect, the transmission/reception antenna pair may be displaced every time the belt is worn to a wrist.

However, Patent Document 1 does not disclose or suggest how a position displacement is to be handled and measured when a position displacement of the transmission/reception antenna pair occurs with respect to the measurement target site. Without any countermeasure, for example, there may be a problem that, in a case where a position displacement of the transmission/reception antenna pair occurs in the circumferential direction of the wrist, the received signal level varies, and the pulse wave as biological information cannot be measured with high accuracy.

Accordingly, an object of the present invention is to provide an antenna device for biological measurement capable of accurately measuring biological information from a measurement target site even when a position displacement of the transmission/reception antenna group occurs with respect to the measurement target site. Another object of the present invention is to provide a pulse wave measuring device, a blood pressure measuring device, and an apparatus provided with the antenna device for biological measurement. Another object of the present invention is to provide a biological information measuring method capable of accurately measuring biological information from a measurement target site even when the position of the transmission/reception antenna group is displaced with respect to the measurement target site. Another object of the present invention is to provide a pulse wave measuring method and a blood pressure measuring method including such a biological information measuring method.

In order to achieve the above object, in a first aspect, an antenna device for biological measurement of the present disclosure is a device that emits radio waves toward a measurement target site of a living body or receives radio waves from the measurement target site to measure biological information, the device comprising:

a belt worn as surrounding a measurement target site of a living body;

a transmission/reception antenna group provided to the belt and including a plurality of antenna elements arranged, in an area where the belt is spread in a strip-like manner, being spaced apart from each other in one direction or two orthogonal directions;

a transmission circuit configured to emit a radio wave toward the measurement target site using any one of antenna elements included in the transmission/reception antenna group as a transmission antenna, in a wearing state where the belt is worn as surrounding an outer surface of the measurement target site;

a reception circuit configured to receive a radio wave reflected from the measurement target site using any one of antenna element included in the transmission/reception antenna group as a reception antenna; and

an antenna control unit configured to weight a transmission/reception antenna pair formed of the transmission antenna and the reception antenna among the plurality of antenna elements based on an output of the reception circuit.

In the present specification, the “measurement target site” may be a trunk in addition to a rod-shaped site such as an upper limb (wrist, upper arm, or the like) or a lower limb (ankle, or the like).

Further, the “outer surface” of the measurement target site refers to a surface exposed to the outside. For example, in a case the measurement target site is a wrist, an outer surface refers to the outer peripheral surface of the wrist or a part thereof (for example, the palmar side surface corresponding to the palm side portion of the outer peripheral surface in the circumferential direction).

Further, the “belt” refers to a band-like member for surrounding the measurement target site, and another term such as “band” may be used.

Further, each “antenna element” refers to an element used as a transmission antenna or a reception antenna, or as a transmission/reception shared antenna via a known circulator.

In addition, the “surface” of the belt spreads in a band-like shape does not indicate whether it is an inner peripheral surface or an outer peripheral surface. The “one direction” in the plane typically refers to the “longitudinal direction” or “width direction” of the belt, but may be a direction obliquely inclined with respect to the “longitudinal direction” or “width direction.” In addition, the “two orthogonal directions” in the plane along the measurement target site of the belt refers to two directions, for example, the “one direction” and a direction orthogonal to the “one direction.” The “longitudinal direction” of the belt corresponds to the circumferential direction of the measurement target site in a wearing state to the measurement target site. The “width direction” of the belt refers to a direction crossing the “longitudinal direction” of the belt.

In addition, to “weight” the transmission/reception antenna pair refers to, for example, that a weight of an antenna element used as a certain transmission/reception antenna pair is set relatively heavy among a plurality of antenna elements, and the weights of other antenna elements are set relatively light.

In this specification, “weight” does not refer to physical weight, but refers to a relative degree (large or small) of usage of each element in a case where a plurality of elements (antenna elements) are used in parallel at the same time.

In a second aspect, an antenna device for biological measurement according to the present disclosure is an antenna device for biological measurement that measures biological information, the device comprising:

a belt worn as surrounding a measurement target site of a living body;

a transmission/reception antenna group provided to the belt and including a plurality of antenna elements arranged, in an area where the belt is spread in a strip-like manner, being spaced apart from each other in one direction or two orthogonal directions;

a transmission circuit configured to emit a radio wave toward the measurement target site using any one of antenna elements included in the transmission/reception antenna group as a transmission antenna, in a wearing state where the belt is worn as surrounding an outer surface of the measurement target site;

a reception circuit configured to receive a radio wave reflected from the measurement target site using any one of antenna element included in the transmission/reception antenna group as a reception antenna;

an antenna control unit configured to select or to weight by switching a transmission/reception antenna pair formed of the transmission antenna and the reception antenna among the plurality of antenna elements based on an output of the reception circuit; and

a storage unit configured to store a signal-to-noise ratio of received signal corresponding to selection or weighting every time the antenna control unit switches the selection or weighting once,

wherein the antenna control unit determines a next selection or weighting based on a signal-to-noise ratio corresponding to past selection or weighting, which is stored in the storage unit, and a signal-to-noise ratio corresponding to the current selection or weighting.

In the present specification, “by switching” is not limited to switching both a transmission antenna and a reception antenna among a plurality of antenna elements and includes, for example, a case where a certain antenna element is fixedly used as the transmission antenna and the reception antenna is switched among a plurality of antenna elements, and a case where a certain antenna element is fixedly used as the reception antenna and the transmission antenna is switched among a plurality of antenna elements.

Further, to “select” a transmission/reception antenna pair refers to, for example, selecting antenna elements used as a certain transmission/reception antenna pair among a plurality of antenna elements and deselecting other antenna elements.

In a third aspect, a pulse wave measuring device according to present disclosure is a pulse wave measuring device that measures a pulse wave at a measurement target site of a living body, the device comprising the antenna device for biological measurement of the second aspect, wherein

the area where the transmission/reception antenna group is provided is placed corresponding to an artery that passes through the measurement target site in the wearing state where the belt is worn as surrounding the outer surface of the measurement target site, and

in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit selects by switching or weights the transmission/reception antenna pair formed of the transmission antenna and the reception antenna among the plurality of antenna elements based on an output from the reception circuit,

further comprising a pulse wave detection unit configured to acquire a pulse wave signal indicating a pulse wave at the artery passing through the measurement target site based on the output from the reception circuit received via the selected or weighted transmission/reception antenna pair.

In a fourth aspect, a blood pressure measuring device according to the present disclosure is a blood pressure measuring device that measures blood pressure at a measurement target site of a living body, the device comprising two sets of pulse wave measuring devices of the third aspect,

wherein the belts of the two sets are integrally formed,

the transmission/reception antenna group of the two sets are arranged spaced apart from each other in a width direction of the belt,

in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,

in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit selects by switching or weights the transmission/reception antenna pair formed of the transmission antenna and the reception antenna among the plurality of antenna elements based on an output from the reception circuit, and

respectively in the two sets, the pulse wave detection unit acquires the pulse wave signal indicating the pulse wave at the artery passing through the measuring site based on the output from the reception circuit received via the selected or weighted transmission/reception antenna pair,

further comprising:

a time difference acquisition unit configured to acquire a time difference between the pulse wave signals respectively acquired by the pulse wave detection unit of the two sets as a pulse wave transit time; and

a first blood pressure calculation unit configured to calculate blood pressure value based on the pulse wave transit time acquired by the time difference acquisition unit using a predetermined correspondence equation between the pulse wave transit time and the blood pressure.

In a fifth aspect, a pulse wave measuring device according to the present disclosure is a device that measures a pulse wave at a measurement target site of a living body, the device comprising the antenna device for biological measurement of the first aspect, wherein

the area where the transmission/reception antenna group is provided is placed corresponding to an artery that passes through the measurement target site in the wearing state where the belt is worn as surrounding the outer surface of the measurement target site, and

in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit weights the transmission/reception antenna pair formed of the transmission antenna and the reception antenna among the plurality of antenna elements based on an output from the reception circuit,

further comprising a pulse wave detection unit configured to acquire a pulse wave signal indicating a pulse wave at the artery passing through the measurement target site based on the output from the reception circuit received via the weighted transmission/reception antenna pair.

In a sixth aspect, a blood pressure measuring device according to the present disclosure is a device that measures blood pressure at a measurement target site of a living body, the device comprising two sets of the pulse wave measuring devices of the fifth aspect,

wherein the belts of the two sets are integrally formed,

the transmission/reception antenna group of the two sets are arranged spaced apart from each other in a width direction of the belt,

in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,

in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit weights the transmission/reception antenna pair formed of the transmission antenna and the reception antenna among the plurality of antenna elements based on an output from the reception circuit, and

respectively in the two sets, the pulse wave detection unit acquires the pulse wave signal indicating the pulse wave at the artery passing through the measuring site based on the output from the reception circuit received via the weighted transmission/reception antenna pair,

further comprising:

a time difference acquisition unit configured to acquire a time difference between the pulse wave signals respectively acquired by the pulse wave detection unit of the two sets as a pulse wave transit time; and

a first blood pressure calculation unit configured to calculate blood pressure value based on the pulse wave transit time acquired by the time difference acquisition unit using a predetermined correspondence equation between the pulse wave transit time and the blood pressure.

In a seventh aspect, an apparatus according to the present disclosure comprises the above-described antenna device for biological measurement, the above-described pulse wave measuring device, or the above-described blood pressure measuring device.

In an eighth aspect, a biological information measuring method according to the present disclosure is a method that measures biological information using a belt to which a transmission/reception antenna group is provided, wherein

the transmission/reception antenna group includes a plurality of antenna elements arranged spaced apart from each other in a longitudinal direction and/or a width direction of the belt,

the biological information measuring method comprising:

wearing the belt as surrounding an outer surface of a measurement target site of the living body into a wearing state so that the transmission/reception antenna group is placed corresponding to an artery passing through the measurement target site; and

in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit.

In a ninth aspect, a pulse wave measuring method according to the present disclosure is a method that measures a pulse wave of a measurement target site of a living body using a belt to which a transmission/reception antenna group is provided, wherein

the transmission/reception antenna group includes a plurality of antenna elements arranged spaced apart from each other in a longitudinal direction and/or a width direction of the belt,

the pulse wave measuring method comprising:

wearing the belt as surrounding an outer surface of a measurement target site into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit; and

acquiring a pulse wave signal indicating a pulse wave at the artery passing through the measurement target site based on the output from the reception circuit received via the weighted transmission/reception antenna pair.

In a tenth aspect, a blood pressure measuring method according to the present disclosure is a method that measures blood pressure at a measurement target site of a living body using a belt to which two sets of transmission/reception antenna groups are integrally provided, wherein

the two sets of the transmission/reception antenna groups are arranged spaced apart from each other in a width direction of the belt and respectively include a plurality of antenna elements arranged spaced apart from each other in a longitudinal direction and/or the width direction of the belt,

the blood pressure measuring method comprising:

wearing the belt as surrounding an outer surface of the measurement target site into a wearing state so that a first set of transmission/reception antenna group of the two sets is placed corresponding to an upstream portion of an artery passing through the measurement target site and a second set of transmission/reception antenna group is placed corresponding to a downstream portion of the artery;

in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, weighting a transmission/reception antenna pair formed of the transmission antenna and the reception antenna among the plurality of antenna elements based on an output from the reception circuit;

respectively in the two sets, acquiring a pulse wave signal indicating a pulse wave at the artery passing through the measurement target site based on the output from the reception circuit received via the weighted transmission/reception antenna pair;

acquiring a time difference between the pulse wave signals respectively received in the two sets as a pulse wave transit time; and

calculating a blood pressure value based on the acquired pulse wave transit time using a predetermined correspondence equation between the pulse wave transit time and the blood pressure.

In an eleven aspect, a biological information measuring method of the present disclosure is a biological information measuring method that measures biological information using a belt to which a transmission/reception antenna group is provided, wherein

the transmission/reception antenna group includes a plurality of antenna elements arranged spaced apart from each other in a longitudinal direction and/or a width direction of the belt,

the biological information measuring method comprising:

wearing the belt as surrounding an outer surface of a measurement target site of a living body into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit,

storing a signal-to-noise ratio of received signal corresponding to selection or weighting in a storage unit every time the selection or weighting is switched once, and

determining a next selection or weighting based on a signal-to-noise ratio corresponding to past selection or weighting stored in the storage unit and a signal-to-noise ratio corresponding to current selection or weighting.

In a twelve aspect, a pulse wave measuring method according to the present disclosure is a pulse wave measuring method that measures a pulse wave at a measurement target site of a living body using a belt to which a transmission/reception antenna group is provided, wherein

the transmission/reception antenna group includes a plurality of antenna elements arranged spaced apart from each other in a longitudinal direction and/or a width direction of the belt,

the pulse wave measuring method comprising:

wearing the belt as surrounding an outer surface of the measurement target site into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;

storing a signal-to-noise ratio of received signal corresponding to selection or weighting in a storage unit every time the selection or weighting is switched once;

determing a next selection or weighting based on a signal-to-noise ratio corresponding to past selection or weighting stored in the storage unit and a signal-to-noise ratio corresponding to current selection or weighting; and

acquiring a pulse wave signal indicating a pulse wave at the artery passing through the measurement target site based on the output from the reception circuit received via the selected or weighted transmission/reception antenna pair.

In a thirteen aspect, a blood pressure measuring method according to the present disclosure is a blood pressure measuring method that measures blood pressure at a measurement target site of a living body using a belt to which two sets of transmission/reception antenna groups are integrally provided, wherein

the two sets of the transmission/reception antenna group are arranged spaced apart from each other in a width direction of the belt and respectively include a plurality of antenna elements arranged spaced apart from each other in a longitudinal direction and/or the width direction of the belt,

the blood pressure measuring method comprising:

wearing the belt as surrounding an outer surface of the measurement target site into a wearing state so that a first set of the transmission/reception antenna group of the two sets is placed corresponding to an upstream portion of an artery passing through the measurement target site and a second set of the transmission/reception antenna group is placed corresponding to a downstream portion of the artery;

in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;

storing a signal-to-noise ratio of received signal corresponding to selection or weighting in a storage unit every time the selection or weighting is switched once;

determing a next selection or weighting based on a signal-to-noise ratio corresponding to past selection or weighting stored in the storage unit and a signal-to-noise ratio corresponding to current selection or weighting;

respectively in the two sets, acquiring a pulse wave signal indicating a pulse wave at the artery passing through the measurement target site based on the output from the reception circuit received via the selected or weighted transmission/reception antenna pair,

acquiring a time difference between the pulse wave signals respectively acquired in the two sets as a pulse wave transit time; and

calculating a blood pressure value based on the acquired pulse wave transit time using a predetermined correspondence equation between the pulse wave transit time and the blood pressure.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a perspective view illustrating an appearance of a wrist sphygmomanometer according to an embodiment of an antenna device for biological measurement, a pulse wave measuring device, and a blood pressure measuring device of the present invention.

FIG. 2 is a diagram schematically illustrating a cross section perpendicular to the longitudinal direction of a wrist in a case where the sphygmomanometer is worn on a left wrist.

FIG. 3 is a diagram illustrating a planar layout of a transmission/reception antenna group constituting first and second pulse wave sensors in a state where the sphygmomanometer is worn on the left wrist.

FIG. 4 is a diagram illustrating an overall block configuration of a control system of the sphygmomanometer.

FIG. 5 is a diagram illustrating a partial and functional block configuration of the control system of the sphygmomanometer.

FIG. 6 is a diagram illustrating a configuration of a transmission antenna switching circuit and a reception antenna switching circuit included in the transmission/reception circuit group of the sphygmomanometer.

FIG. 7A is a diagram schematically illustrating a cross-section along the longitudinal direction of the wrist in a state where the sphygmomanometer is worn to the left wrist. FIG. 7B is a diagram illustrating waveforms of first and second pulse wave signals output from the first and second pulse wave sensors, respectively.

FIG. 8A is a diagram illustrating a block configuration implemented by a program for performing an oscillometric method in the sphygmomanometer.

FIG. 8B is a diagram illustrating an operation flow in a case where the sphygmomanometer performs blood pressure measurement by the oscillometric method.

FIG. 9 is a diagram illustrating changes in cuff pressure and pulse wave signals caused by the operation flow of FIG. 8B.

FIG. 10 is an overall operation flow according to a biological information measuring method, a pulse wave measuring method, and a blood pressure measuring method according to one embodiment of the present invention, in which the sphygmomanometer performs pulse wave measurement to acquire a pulse wave transit time (PTT) and performs blood pressure measurement (estimation) based on the pulse wave transit time.

FIGS. 11A to 11D are diagrams each illustrating a mode how a transmission/reception antenna group mounted on a belt is displaced with respect to the wrist.

FIG. 12A is a diagram illustrating an operation flow of a method for selecting by switching a transmission/reception antenna pair by a CPU of the sphygmomanometer. FIG. 12B is a diagram illustrating a modification of the operation flow in FIG. 12A.

FIG. 13A is a diagram illustrating a waveform (S/N=34 dB) of a pulse wave signal acquired as a result of position displacement of the transmission/reception antenna group with respect to a radial artery in the longitudinal direction of the belt.

FIG. 13B is a diagram illustrating a waveform (S/N=47 dB) of the pulse wave signal acquired by the operation flow of FIG. 12.

FIG. 14 illustrates a partial and functional block configuration of a control system in a case where the sphygmomanometer includes a transmission antenna weighting and phase shift circuit and a reception antenna weighting and phase shift circuit, in contrast to FIG. 5.

FIG. 15 is a diagram illustrating a configuration of the transmission antenna weighting and phase shift circuit and the reception antenna weighting and phase shift circuit.

FIG. 16A is a diagram illustrating an operation flow of a method of weighting a transmission/reception antenna pairs by the CPU of the sphygmomanometer.

FIG. 16B is a diagram illustrating the operation flow of the method of weighting the transmission/reception antenna pairs by the CPU of the sphygmomanometer.

FIG. 16C is a diagram illustrating the operation flow of the method of weighting the transmission/reception antenna pairs by the CPU of the sphygmomanometer.

FIGS. 17A to 17H schematically illustrates weighting states in a first set of transmission/reception antenna pairs and a second set of transmission/reception antenna pairs in accordance with the operation flows of FIGS. 16A to 16C.

FIG. 18A is a diagram illustrating an operation flow in a case where the CPU controls a function A described in FIGS. 16A to 16C.

FIG. 18B is a diagram illustrating the operation flow in a case where the CPU controls the function A described in FIGS. 16A to 16C.

FIG. 19A is a diagram illustrating an operation flow in a case where the CPU controls a function C described in FIGS. 16A to 16C.

FIG. 19B is a diagram illustrating the operation flow in a case where the CPU controls the function C described in FIGS. 16A to 16C.

FIG. 20A is a diagram illustrating an operation flow in a case where the CPU of the sphygmomanometer weights with respect to transmission/reception antennas of two rows and two columns.

FIG. 20B is a diagram illustrating the operation flow in a case where the CPU of the sphygmomanometer weights the transmission/reception antennas in two rows and two columns.

FIG. 20C is a diagram illustrating the operation flow in the case where the CPU of the sphygmomanometer weights the transmission/reception antennas in two rows and two columns.

FIGS. 21A to 21I schematically illustrate weighting states in the first sets of transmission/reception antenna pairs and the second set of transmission/reception antenna pairs in accordance with the operation flow of FIGS. 20A to 20C.

FIG. 22A is a diagram illustrating an operation flow in a case where the CPU controls a function B described in FIGS. 20A to 20C.

FIG. 22B is a diagram illustrating the operation flow in a case where the CPU controls the function B described in FIGS. 20A to 20C.

FIG. 23A is a diagram illustrating an operation flow of a method of dynamically searching for a transmission/reception antenna pair suitable for use by the CPU of the sphygmomanometer.

FIG. 23B is a diagram illustrating the operation flow of the method of dynamically searching for a transmission/reception antenna pair suitable for use by the CPU of the sphygmomanometer.

FIG. 23C is a diagram illustrating the operation flow of the method of dynamically searching for a transmission/reception antenna pair suitable for use by the CPU of the sphygmomanometer.

FIGS. 24A to 24F are diagrams illustrating modifications of the second set of transmission/reception antenna pairs (and the first set of transmission/reception antenna pairs).

FIGS. 25A and 25B are diagrams illustrating another modification of the second set of transmission/reception antenna pairs (and the first set of transmission/reception antenna pairs).

FIGS. 26A to 26C are diagrams illustrating still another modification of the second set of transmission/reception antenna pairs (and the first set of transmission/reception antenna pairs).

FIG. 27 is a diagram illustrating another planar layout of the transmission/reception antenna group constituting the first and second pulse wave sensors in a state where the sphygmomanometer is worn to the left wrist, as compared with FIG. 3.

FIG. 28A is an enlarged view illustrating one antenna element in FIG. 3.

FIGS. 28B and 28C are diagrams illustrating modifications of the antenna element.

### DESCRIPTION OF EMBODIMENT

Hereinafter, an embodiment of the present invention will be described in detail with reference to the drawings.

### (Configuration of Sphygmomanometer)

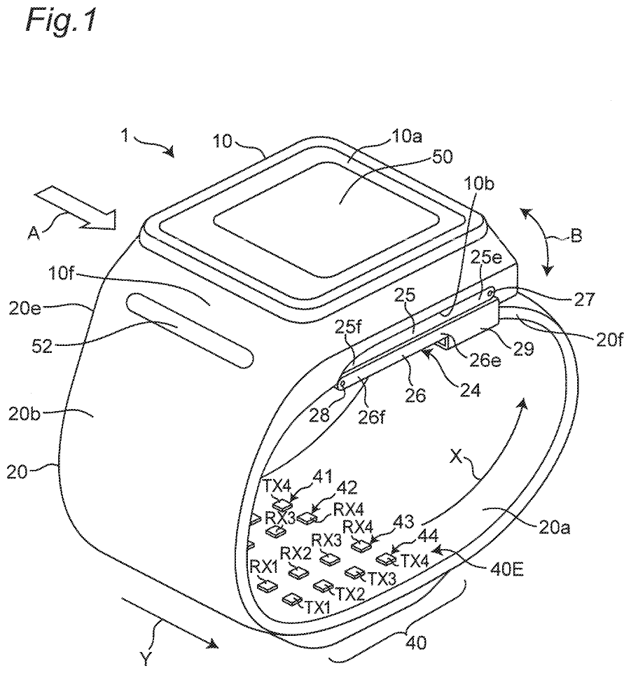

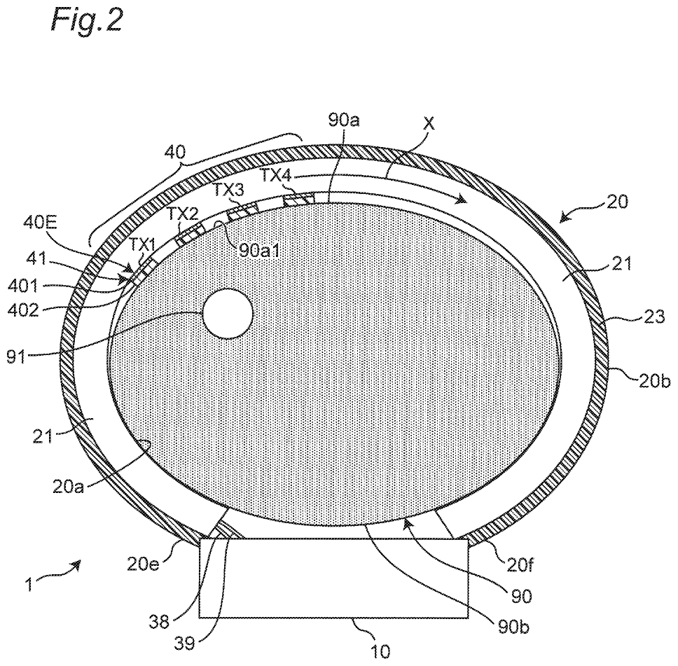

FIG. 1 illustrates a perspective view of an appearance of a wrist sphygmomanometer (the whole body is indicated by reference numeral 1) according to an embodiment of an antenna device for biological measurement, a pulse wave measuring device, and a blood pressure measuring device of the present invention. FIG. 2 schematically illustrates a cross section perpendicular to a longitudinal direction of a left wrist 90 in a state where the sphygmomanometer 1 is worn on the left wrist 90 as a measurement target site (hereinafter, referred to as “wearing state”).

As illustrated in the drawings, the sphygmomanometer 1 roughly includes a belt 20 to be worn so as to surround the user's left wrist 90 and a main body 10 integrally fitted to the belt 20. This sphygmomanometer 1 is configured as a whole corresponding to a blood pressure measuring device including two sets of pulse wave measuring devices. Each pulse wave measuring device includes an antenna device for biological measurement.

As can be seen from FIG. 1, the belt 20 has an elongated band-like shape so as to surround the left wrist 90 along a circumferential direction, an inner peripheral surface 20a being in contact with the left wrist 90, and an outer peripheral surface 20b in an opposite side of the inner peripheral surface 20a. The dimension (width dimension) in the width direction Y of the belt 20 is set to about 30 mm in this example.

The main body 10 is integrally provided at one end 20e in the circumferential direction of the belt 20 by being integrally formed in this example. Note that the belt 20 and the main body 10 may be formed separately, and the main body 10 may be integrally attached to the belt 20 via an engaging member (a hinge, for example). In this example, a site where the main body 10 is arranged is supposed to correspond to a back side surface (a surface on a back side of a hand) 90b of a left wrist 90 in the wearing state (see FIG. 2). FIG. 2 illustrates a radial artery 91 passing through near a palmar side surface (a surface on a palm side) 90a as an outer surface in the left wrist 90.

As can be seen from FIG. 1, the main body 10 has a three-dimensional shape having a thickness in a direction perpendicular to the outer peripheral surface 20b of the belt 20. The main body 10 is formed to be compact and thin so as not to disturb user's daily activities. In this example, the main body 10 has a contour having a truncated quadrangular pyramid shape projecting outward from the belt 20.

A display unit 50 serving as a display screen is provided on a top surface (a surface farthest from a measurement target site) 10a of the main body 10. Further, an operation unit 52 for inputting an instruction from the user is provided along a side surface (a side surface on a left front side in FIG. 1) 10f of the main body 10.

A transmission/reception unit 40 constituting first and second pulse wave sensors is provided on a site of the belt 20 between one end 20e and an other end 20f in the circumferential direction. On the inner peripheral surface 20a of the site of the belt 20 where the transmission/reception unit 40 is arranged, a transmission/reception antenna group 40E including a plurality of antenna elements TX1, TX2, . . . , RX1, RX2, which are arranged by being spaced apart from each other with respect to the longitudinal direction X and the width direction Y of the belt 20, is mounted (described in detail later). In this example, a range where the transmission/reception antenna group 40E is provided in the longitudinal direction X of the belt 20 is supposed to correspond to the radial artery 91 of the left wrist 90 in the wearing state (see FIG. 2).

As illustrated in FIG. 1, a bottom surface (a surface closest to the measurement target site) 10b of the main body 10 and the end 20f of the belt 20 are connected by a threefold buckle 24. The buckle 24 includes a first plate-like member 25 arranged on an outer peripheral side and a second plate-like member 26 arranged on an inner peripheral side. One end portion 25e of the first plate-like member 25 is rotatably fitted to the main body 10 via a connecting rod 27 extending along the width direction Y. An other end portion 25f of the first plate-like member 25 is rotatably fitted to one end portion 26f of the second plate-like member 26 via a connecting rod 28 extending along the width direction Y. An other end portion 26e of the second plate-like member 26 is fixed in the neighborhood of the end portion 20f of the belt 20 by a fixing portion 29. Note that the fitting position of the fixing portion 29 in the longitudinal direction X of the belt 20 (corresponding to the circumferential direction of the left wrist 90 in the wearing state) is variably set in advance according to the circumferential length of the left wrist 90 of the user. Thus, the sphygmomanometer 1 (belt 20) is configured in a substantially annular shape as a whole, and the bottom surface 10b of the main body 10 and the end portion 20f of the belt 20 can be opened and closed by the buckle 24 in the direction of arrow B.

When the user wears the sphygmomanometer 1 on the left wrist 90, the user inserts his or her left hand through the belt 20 in a direction indicated by arrow A in FIG. 1 in a state where the buckle 24 is opened to increase a diameter of a ring formed by the belt 20. Then, as illustrated in FIG. 2, the user adjusts an angular position of the belt 20 around the left wrist 90 to position the transmission/reception unit 40 of the belt 20 on the radial artery 91 passing through the left wrist 90. As a result, the transmission/reception antenna group 40E of the transmission/reception unit 40 is set to contact with a portion 90al of the palmar side surface 90a of the left wrist 90 which meets the radial artery 91. In this state, the user closes and fixes the buckle 24. Thus, the user wears the sphygmomanometer 1 (belt 20) on the left wrist 90.

As illustrated in FIG. 2, in this example, the belt 20 includes a strip 23 forming an outer peripheral surface 20b, and a pressing cuff 21 as a press member attached along the inner peripheral surface of the strip 23. The strip 23 is made of a plastic material (silicone resin, in this example) which is flexible in the thickness direction and substantially non-stretchable in the longitudinal direction X (corresponding to the circumferential direction of the left wrist 90) (substantially no elastic property), in this example. In this example, the pressing cuff21 is configured as a fluid bag by confronting two stretchable polyurethane sheets in the thickness direction and welding peripheral edge portions thereof. As described above, the transmission/reception antenna group 40E of the transmission/reception unit 40 is arranged at a site of the inner peripheral surface 20a of the pressing cuff 21 (belt 20) which meets the radial artery 91 of the left wrist 90.

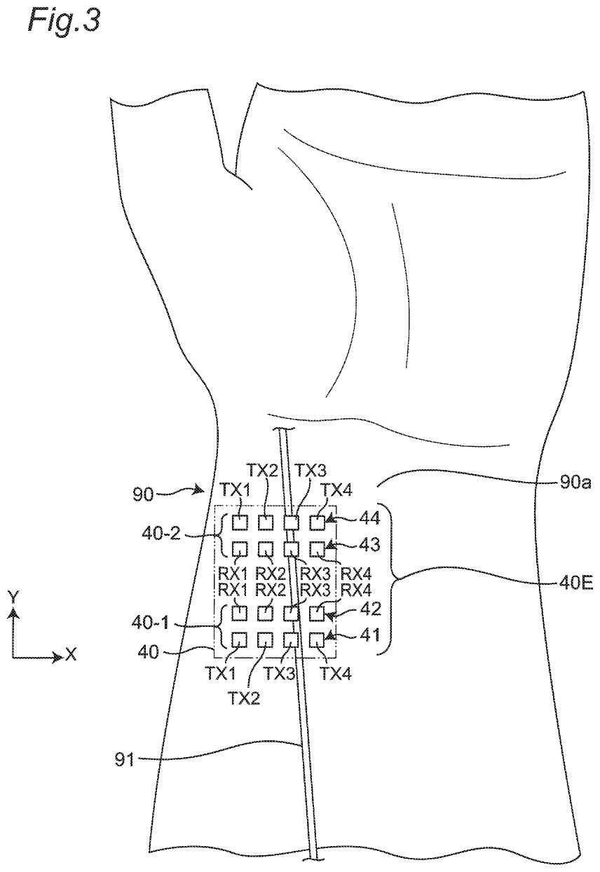

As illustrated in FIG. 3, the transmission/reception antenna group 40E of the transmission/reception unit 40 includes two transmission antenna arrays 41 and 44 and two reception antenna arrays 42 and 43 respectively arranged in rows along the circumferential direction of the left wrist 90 (corresponding to the longitudinal direction X of the belt 20) as being separated from each other roughly along the longitudinal direction of the left wrist 90 (corresponding to the width direction Y of the belt 20) corresponding to the radial artery 91 of the left wrist 90 in the wearing state. In this example, in the width direction Y, the transmission antenna arrays 41 and 44 are arranged on opposite sides within an area where the transmission/reception antenna group 40E is provided, and the reception antenna arrays 42 and 43 are arranged between these transmission antenna arrays 41 and 44. Each of the transmission antenna arrays 41 and 44 includes four antenna elements TX1, TX2, TX3, and TX4 used as transmission antennas in a state of being spaced apart from each other along the longitudinal direction X (hereinafter, these antenna elements are referred to as transmission antennas TX1, TX2, TX3, and TX4). Each of the reception antenna arrays 42 and 43 includes four antenna elements RX1, RX2, RX3, and RX4 used as reception antennas in a state of being spaced apart from each other along the longitudinal direction X (hereinafter, these antenna elements are referred to as reception antennas RX1, RX2, RX3, and RX4). The transmission antennas TX1, TX2, TX3, and TX4 included in the transmission antenna array 41 and the reception antennas RX1, RX2, RX3, and RX4 which are included in adjacent reception antenna array 42 and respectively receive radio waves from the transmission antennas TX1, TX2, TX3, and TX4 form a first set of transmission/reception antenna pairs (TX1, RX1), (TX2, RX2), (TX3, RX3), and (TX4, RX4) (each pair is expressed in parentheses. These pairs are collectively referred to as “first set of transmission/reception antenna pairs (41, 42)”.). In a similar manner, the transmission antennas TX1, TX2, TX3, and TX4 included in the transmission antenna array 44 and the reception antennas RX1, RX2, RX3, and RX4 which are included in adjacent reception antenna array 43 and respectively receive radio waves from the transmission antennas TX1, TX2, TX3, and TX4 form a second set of transmission/reception antenna pairs (TX1, RX1), (TX2, RX2), (TX3, RX3), and (TX4, RX4) (Each pair is expressed in parentheses. These pairs are collectively referred to as “second set of transmission/reception antenna pairs (44, 43)”.). In this arrangement, the transmission antenna array 41 is closer to the reception antenna array 42 than the transmission antenna array 44 in the width direction Y. Further, the transmission antenna array 44 is closer to the reception antenna array 43 than the transmission antenna array 41 in the width direction Y. Therefore, interference between the first set of transmission/reception antenna pairs (41, 42) and the second set of transmission/reception antenna pairs (44, 43) can be reduced. Also, in the first set of transmission/reception antenna pairs (41, 42) and the second set of transmission/reception antenna pairs (44, 43), respectively, along the width direction Y of the belt 20, the transmission/reception antenna pairs (TX1, RX1), (TX2, RX2), (TX3, RX3), and (TX4, RX4) are arranged side by side apart from each other, so transmission/reception antenna pairs (TX1, RX1), (TX2, RX2), (TX3, RX3), and (TX4, RX4) can transmit and receive simultaneously without using a circulator.

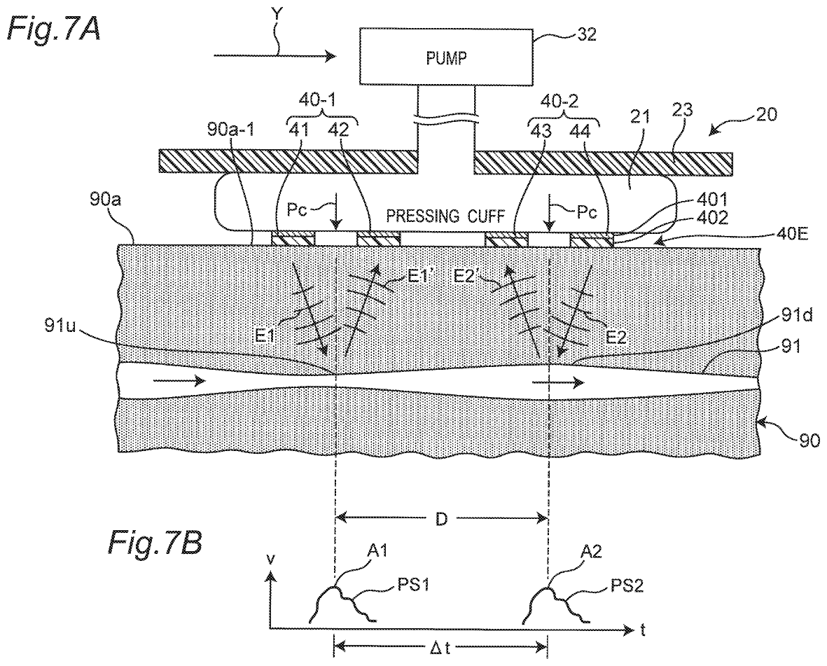

In this example, one transmission antenna or one reception antenna is in a square pattern shape having approximately 3 mm in both vertical and horizontal directions with respect to the plane direction (that is the direction of the paper surface of FIG. 3) so that radio waves having a frequency of 24 GHz band can be emitted or received. In the width direction Y of the belt 20, the distance between the center of the transmission antennas TX1, TX2, TX3, and TX4 and the center of the adjacent reception antennas RX1, RX2, RX3, and RX4 in the first set is in a range of 8 mm to 10 mm. In a similar manner, in the width direction Y of the belt 20, the distance between the center of the transmission antennas TX1, TX2, TX3, and TX4 and the center of each of the adjacent reception antennas RX1, RX2, RX3, and RX4 in the second set is in a range of 8 mm to of 10 mm. Further, in the width direction Y of the belt 20, a distance D between the center of the first set of transmission/reception antenna pairs (41, 42) and the center of the second set of transmission/reception antenna pairs (44, 43) (see FIG. 7A) is set to 20 mm in this example. This distance D corresponds to a substantial distance between the first set of transmission/reception antenna pairs (41, 42) and the second set of transmission/reception antenna pairs (44, 43).

Further, as illustrated in FIG. 2, in this example, each of the transmission antennas TX1, TX2, TX3, and TX4 has a conductor layer 401 for emitting radio waves. A dielectric layer 402 is attached along the surface of the conductor layer 401 in a part facing the left wrist 90 (the same configuration is used for each transmission antenna and reception antenna). In this example, the pattern shape of the dielectric layer 402 is set to be the same as the pattern shape of the conductor layer 401; however, different pattern shapes may be used. In a wearing state in which the transmission/reception antenna group 40E is worn to the left wrist 90, a surface of the dielectric layer 402 opposite to a surface attached to the conductor layer 401 contacts with the palmar side surface 90a of the left wrist 90. In this wearing state, the conductor layer 401 faces the palmar side surface 90a of the left wrist 90, and the dielectric layer 402 serves as a spacer to increase the distance between the palmar side surface 90a of the left wrist 90 and the conductor layer 401. With this configuration, biological information from the left wrist 90 can be accurately measured.

In this example, the conductor layer 401 is made of metal (copper, for example). In this example, the dielectric layer 402 is made of polycarbonate, so that the dielectric constant of the dielectric layer 402 is uniformly set to εᵣ≈3.0. Note that the dielectric constant means a dielectric constant at a frequency of 24 GHz band of radio waves used for transmitting and receiving.

Such a transmission/reception antenna group 40E can be configured to be flat along the surface direction. Therefore, in the sphygmomanometer 1, the belt 20 as a whole can be made thin.

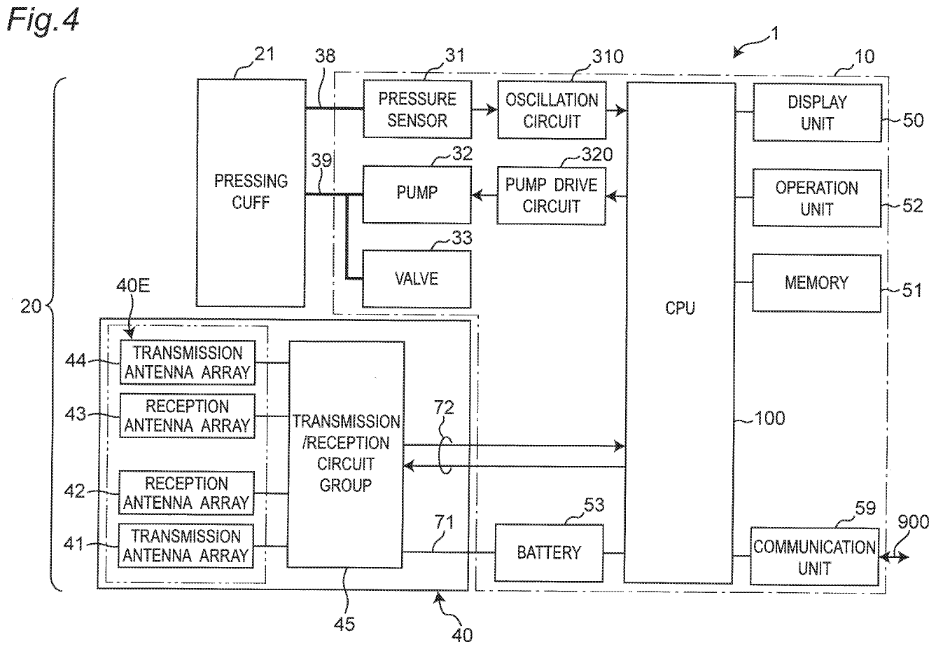

FIG. 4 illustrates an overall block configuration of a control system of the sphygmomanometer 1. In the main body 10 of the sphygmomanometer 1, in addition to the above-described display unit 50 and operation unit 52, a central processing unit (CPU) 100 as a control unit, a memory 51 as a storage unit, a communication unit 59, a pressure sensor 31, a pump 32, a valve 33, an oscillation circuit 310 that converts an output from the pressure sensor 31 into a frequency, and a pump drive circuit 320 that drives the pump 32 are mounted. Furthermore, in addition to the above-described transmission/reception antenna group 40E, the transmission/reception unit 40 includes a transmission/reception circuit group 45 that is controlled by the CPU 100 executing a predetermined program stored in the memory 51.

In this example, the display unit 50 is formed of an organic electro luminescence (EL) display, and displays information related to blood pressure measurement such as a blood pressure measurement result and other information according to a control signal from the CPU 100. Here, the display unit 50 is not limited to an organic EL display, and may be another type of display device such as a liquid crystal display (LCD).

In this example, the operation unit 52 is configured by a push-type switch, and inputs an operation signal according to an instruction to start or stop blood pressure measurement by the user to the CPU 100. Note that the operation unit 52 is not limited to a push-type switch, and may be, for example, a pressure-sensitive (resistance) or proximity (capacitance) touch panel switch. In addition, a microphone (not illustrated) may be provided, and a blood pressure measurement start instruction may be input by a user's voice.

The memory 51 stores data of a program for controlling the sphygmomanometer 1, data used for controlling the sphygmomanometer 1, setting data for setting various functions of the sphygmomanometer 1, data of blood pressure value measurement results, and the like on a non-transitory basis. Further, the memory 51 is used as a work memory when the program is executed.

The CPU 100 executes, as a control unit, various functions in accordance with the program for controlling the sphygmomanometer 1 stored in the memory 51. For example, when executing blood pressure measurement by the oscillometric method, the CPU 100 controls to drive the pump 32 (and the valve 33) based on a signal from the pressure sensor 31 in response to an instruction to start blood pressure measurement from the operation unit 52. Here, in this example, the CPU 100 performs control to calculate the blood pressure value based on the signal from the pressure sensor 31.

The communication unit 59 is controlled by the CPU 100 to transmit predetermined information to an external device via a network 900, or receive information from the external device via the network 900 and transfer the data to the CPU 100. Communication via the network 900 may be performed by either wireless or wired. The network 900 is the Internet in this embodiment; however this does not set any limitation, and other types of network such as an in-hospital local area network (LAN) or one-to-one communication using a USB cable or the like may be used. The communication unit 59 may include a micro USB connector.

The pump 32 and the valve 33 are connected to the pressing cuff21 via an air pipe 39 and the pressure sensor 31 is connected to the pressing cuff 21 via an air pipe 38. Note that the air pipes 39 and 38 may be a single common pipe. The pressure sensor 31 detects the pressure in the pressing cuff 21 via the air pipe 38. In this example, the pump 32 is a piezoelectric pump, and supplies air as a pressing fluid to the pressing cuff 21 through the air pipe 39 in order to increase the pressure (cuff pressure) in the pressing cuff21. The valve 33 is mounted on the pump 32 and is configured to be opened and closed as the pump 32 is turned on/off. In other words, when the pump 32 is turned on, the valve 33 is closed to enclose air in the pressing cuff21 and, when the pump 32 is turned off, the valve 33 is open to discharge the air in the pressing cuff 21 to the atmosphere through the air pipe 39. The valve 33 has a check valve function, and the discharged air does not flow backward. The pump drive circuit 320 drives the pump 32 based on a control signal provided from the CPU 100.

The pressure sensor 31 is a piezoresistive pressure sensor in this example, and detects the pressure of the belt 20 (pressing cuff 21) through the air pipe 38, which is the pressure based on atmospheric pressure as a reference (zero) in this example, and outputs detected results as time series signal. The oscillation circuit 310 oscillates according to an electric signal value based on a change in electric resistance due to the piezoresistance effect from the pressure sensor 31, and outputs a frequency signal having a frequency corresponding to the electric signal value of the pressure sensor 31 to the CPU 100. In this example, the output from the pressure sensor 31 is used to control the pressure in the pressing cuff21 and to calculate blood pressure values including systolic blood pressure (SBP) and diastolic blood pressure (DBP) by the oscillometric method.

A battery 53 supplies power to elements mounted on the main body 10, which are, in this example, each element of the CPU 100, the pressure sensor 31, the pump 32, the valve 33, the display unit 50, the memory 51, the communication unit 59, the oscillation circuit 310, and the pump drive circuit 320. Further, the battery 53 also supplies power to the transmission/reception circuit group 45 of the transmission/reception unit 40 through a wiring 71. This wiring 71, together with a signal wiring 72, is provided being sandwiched between the strip 23 of the belt 20 and the pressing cuff 21, and extending along the longitudinal direction X of the belt 20 between the main body 10 and the transmission/reception unit 40.

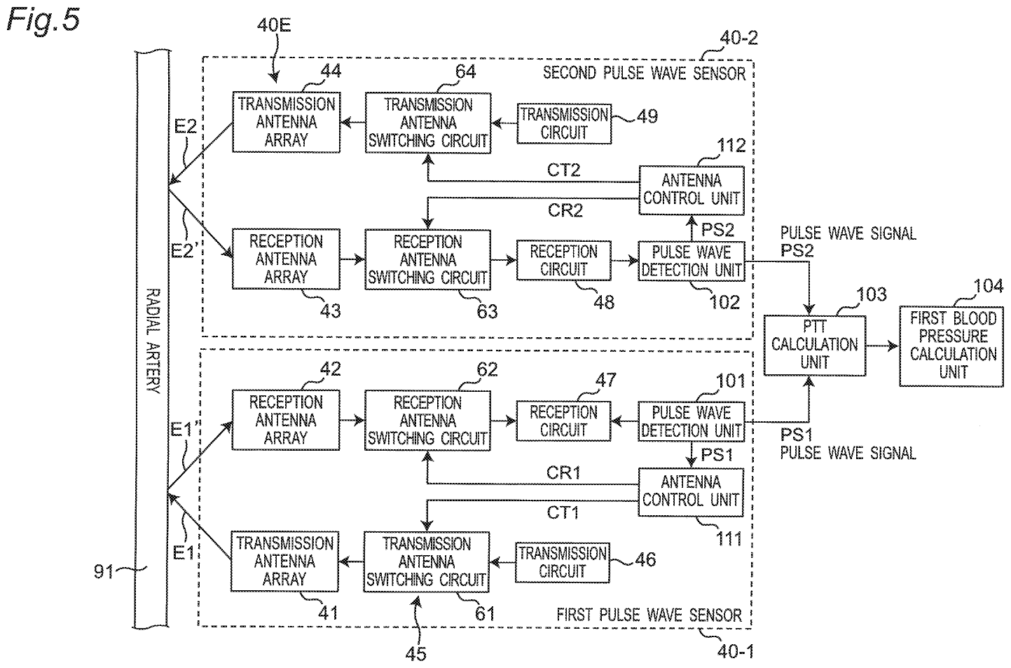

As illustrated in FIG. 5, the transmission/reception circuit group 45 of the transmission/reception unit 40 includes transmission antenna switching circuits 61 and 64 respectively connected to the transmission antenna arrays 41 and 44, transmission circuits 46 and 49 respectively connected to the transmission antenna switching circuits 61 and 64, reception antenna switching circuits 62 and 63 respectively connected to the reception antenna arrays 42 and 43, and reception circuits 47 and 48 respectively connected to the reception antenna switching circuits 62 and 63. In the operation, the transmission circuits 46 and 49 respectively emit radio waves E1 and E2 having a frequency of 24 GHz band in this example via the transmission antenna switching circuits 61 and 64 and the transmission antenna arrays 41 and 44. The reception circuits 47 and 48 respectively receive radio waves E1′ and E2′ reflected by the left wrist 90 (more precisely, the corresponding part of the radial artery 91) as the measurement target site via the reception antenna arrays 42 and 43 and the reception antenna switching circuits 62 and 63 to detect and amplify the waves. The transmission antenna switching circuits 61 and 64 and the reception antenna switching circuits 62 and 63 may be realized by hardware such as a switching element, or may be realized by software by a program in the CPU 100.

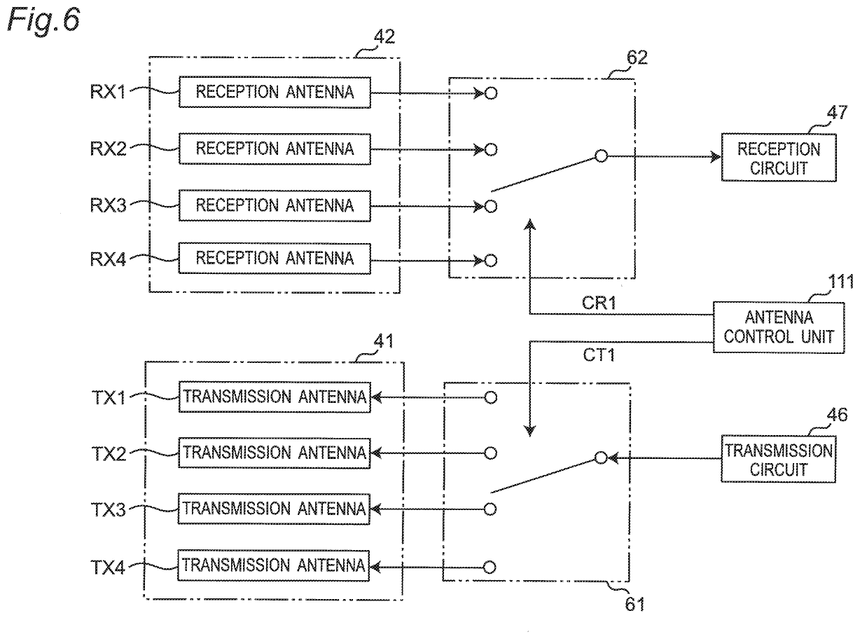

In this example, as schematically illustrated in FIG. 6, the transmission antenna switching circuit 61 may function as a one-circuit/four-contact changeover switch, and select a transmission antenna to be used from the transmission antennas TX1, TX2, TX3, and TX4 included in the transmission antenna array 41 according to the transmission antenna control signal CT1 from the antenna control unit 111. In a similar manner, the reception antenna switching circuit 62 functions as a one-circuit/four-contact changeover switch, and selects a reception antenna to be used from the reception antennas RX1, RX2, RX3, and RX4 included in the reception antenna array 42 according to the reception antenna control signal CR1 from the antenna control unit 111. In this example, the transmission antenna switching circuit 61 and the reception antenna switching circuit 62 are switched in conjunction with each other, and a transmission/reception antenna pair (TXi, RXi) (where i is any one of 1, 2, 3, and 4) to be used is selected from the transmission/reception antenna pairs (TX1, RX1), (TX2, RX2), (TX3, RX3), and (TX4, RX4) included in the first set of transmission/reception antenna pairs (41, 42). However, when m is any one of 1, 2, 3, and 4, n is any one of 1, 2, 3, and 4, and m is not equal to n, such as a combination (TX1, RX2) or the like, a combination of transmission/reception antenna pair (TXm, RXn) is also available.

Further, the transmission antenna switching circuit 64 illustrated in FIG. 5 is configured in the similar manner as the transmission antenna switching circuit 61, and in accordance with the transmission antenna control signal CT2 from the antenna control unit 112, a transmission antenna to be used is selected from the transmission antennas TX1, TX2, TX3, and TX4 included in the transmission antenna array 44. Further, the reception antenna switching circuit 63 is configured in a similar manner as the reception antenna switching circuit 62, and in accordance with the reception antenna control signal CR2 from the antenna control unit 112, a reception antenna to be used is selected from the reception antennas RX1, RX2, RX3, and RX4 included in the reception antenna array 43. In this example, the transmission antenna switching circuit 64 and the reception antenna switching circuit 63 are switched in conjunction with each other, and a transmission/reception antenna pair (TXj, RXj) (where j=1, 2, 3, or 4) to be used is selected from the transmission/reception antenna pairs (TX1, RX1), (TX2, RX2), (TX3, RX3), and (TX4, RX4) included in the second set of transmission/reception antenna pairs (44, 43). However, when m is any one of 1, 2, 3, and 4, n is any one of 1, 2, 3, and 4, and m is not equal to n, such as a combination (TX1, RX2) or the like, a combination of transmission/reception antenna pair (TXm, RXn) is also available.

As will be described in detail later, pulse wave detection units 101 and 102 illustrated in FIG. 5 respectively acquire pulse wave signals PS1 and PS2 indicating pulse waves at the radial artery 91 passing through the left wrist 90 based on the outputs of the reception circuits 47 and 48. Based on the pulse wave signal PS1 from the pulse wave detection unit 101, the antenna control unit 111 outputs a transmission antenna control signal CT1 and a reception antenna control signal CR1 for selecting a transmission/reception antenna pair to be used from the transmission/reception antenna pairs (TX1, RX1), (TX2, RX2), (TX3, RX3), and (TX4, RX4) included in the first set of transmission/reception antenna pairs (41, 42). In a similar manner, based on the pulse wave signal PS2 from the pulse wave detection unit 102, the antenna control unit 112 outputs a transmission antenna control signal CT2 and a reception antenna control signal CR2 for selecting a transmission/reception antenna pair to be used from the transmission/reception antenna pairs (TX1, RX1), (TX2, RX2), (TX3, RX3), and (TX4, RX4) included in the second set of transmission/reception antenna pairs (44, 43). Furthermore, a PTT calculation unit 103 as a time difference acquisition unit acquires a time difference between the pulse wave signals PS1 and PS2 respectively acquired by the two sets of pulse wave detection units 101 and 102 as a pulse wave transit time (PTT). Further, the first blood pressure calculation unit 104 calculates a blood pressure value based on the pulse wave transit time acquired by the PTT calculation unit 103 using a predetermined correspondence equation between the pulse wave transit time and the blood pressure. Here, the pulse wave detection units 101 and 102, the antenna control units 111 and 112, the PTT calculation unit 103, and the first blood pressure calculation unit 104 are realized by the CPU 100 executing a predetermined program stored in the memory 51. The transmission antenna array 41, the reception antenna array 42, the transmission antenna switching circuit 61, the reception antenna switching circuit 62, the transmission circuit 46, the reception circuit 47, the pulse wave detection unit 101, and the antenna control unit 111 configure a first pulse wave sensor 40-1 as the first set of pulse wave measuring devices. The transmission antenna array 44, the reception antenna array 43, the transmission antenna switching circuit 64, the reception antenna switching circuit 63, the transmission circuit 49, the reception circuit 48, the pulse wave detection unit 102, and the antenna control unit 112 configure a second pulse wave sensor 40-2 as a second set of pulse wave measuring devices.

In the wearing state, as illustrated in FIG. 7A, in the longitudinal direction of the left wrist 90 (corresponding to the width direction Y of the belt 20), the first set of transmission/reception antenna pairs (41, 42) corresponds to an upstream portion 91u of the radial artery 91 passing through the left wrist 90, and the second set of transmission/reception antenna pairs (44, 43) corresponds to a downstream portion 91d of the radial artery 91. The signal acquired by the first transmission/reception antenna pair (41, 42) indicates a change in distance corresponding to pulse waves (which leads expansion and contraction of blood vessels) between the upstream portion 91u of the radial artery 91 and the first set of transmission/reception antenna pairs (41, 42). The signal acquired by the second transmission/reception antenna pair (44, 43) indicates a change in distance corresponding to pulse waves between the downstream portion 91d of the radial artery 91 and the second set of transmission/reception antenna pairs (44, 43). The pulse wave detection unit 101 of the first pulse wave sensor 40-1 and the pulse wave detection unit 102 of the second pulse wave sensor 40-2 respectively output the first pulse wave signal PS1 and the second pulse wave signal PS2 in time series, which respectively have a mountain-like waveform as illustrated in FIG. 7B based on the outputs of the reception circuits 47 and 48.

In this example, the reception levels of the reception antenna arrays 42 and 43 are about 1 μW (−30 dBm in decibel value for 1 mW). The output levels of the reception circuits 47 and 48 are about 1 volt. Further, respective peaks A1 and A2 of the first pulse wave signal PS1 and the second pulse wave signal PS2 are about 100 mV to 1 volt.

Note that, in a case where the pulse wave velocity (PWV) of the blood flow in the radial artery 91 is in a range of 1000 cm/s to 2000 cm/s, since a substantial distance D between the first pulse wave sensor 40-1 and the second pulse wave sensor 40-2 is 20 mm, a time difference Δt between the first pulse wave signal PS1 and the second pulse wave signal PS2 is in a range of 1.0 ms to 2.0 ms.

### (Configuration and Operation of Blood Pressure Measurement by Oscillometric Method)

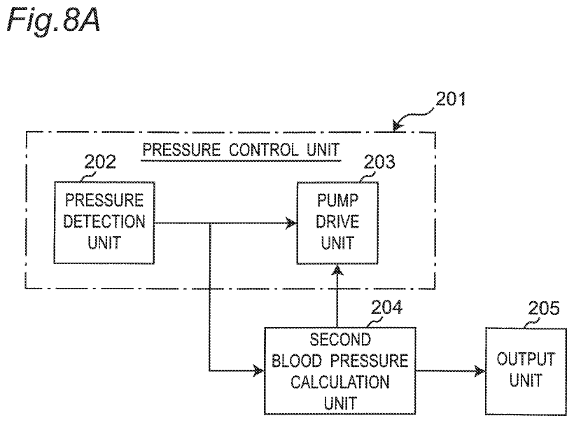

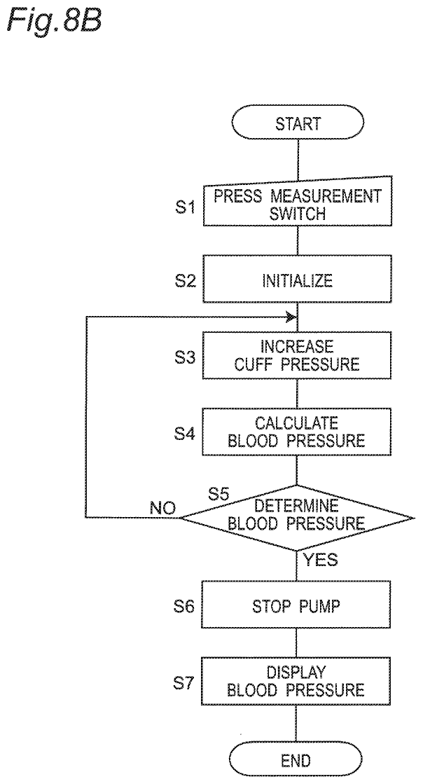

FIG. 8A illustrates a block configuration implemented by a program for performing the oscillometric method in the sphygmomanometer 1.

In this block configuration, roughly, a pressure control unit 201, a second blood pressure calculation unit 204, and an output unit 205 are mounted.

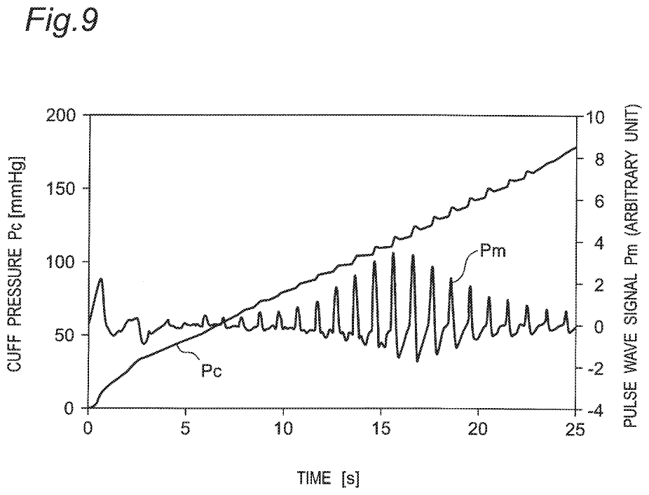

The pressure control unit 201 further includes a pressure detection unit 202 and a pump drive unit 203. The pressure detection unit 202 processes the frequency signal input from the pressure sensor 31 through the oscillation circuit 310, and performs processing for detecting the pressure (cuff pressure) in the pressing cuff21. The pump drive unit 203 performs a process for driving the pump 32 and the valve 33 through the pump drive circuit 320 based on the detected cuff pressure Pc (see FIG. 9). Thereby, the pressure control unit 201 controls the pressure by supplying air to the pressing cuff 21 at a predetermined pressing speed.

The second blood pressure calculation unit 204 acquires a fluctuation component of the arterial volume included in the cuff pressure Pc as a pulse wave signal Pm (see FIG. 9), and based on the acquired pulse wave signal Pm, calculates a blood pressure values (systolic blood pressure SBP and diastolic blood pressure DBP) by applying a known algorithm using the oscillometric method. When the calculation of the blood pressure value is completed, the second blood pressure calculation unit 204 stops the processing of the pump drive unit 203.

The output unit 205 performs processing for displaying the calculated blood pressure values (systolic blood pressure SBP and diastolic blood pressure DBP) on the display unit 50 in this example.

FIG. 8B illustrates an operation flow (blood pressure measuring method flow) when the sphygmomanometer 1 performs blood pressure measurement by the oscillometric method. The belt 20 of the sphygmomanometer 1 is assumed to be worn in advance so as to surround the left wrist 90.

When the user instructs blood pressure measurement by the oscillometric method using a push-type switch as the operation unit 52 provided in the main body 10 (step S1), the CPU 100 starts operation and initializes the processing memory area (step S2). Further, the CPU 100 turns off the pump 32 via the pump drive circuit 320, opens the valve 33, and exhausts the air in the pressing cuff 21. Subsequently, control is performed to set a current output value of the pressure sensor 31 as a value corresponding to the atmospheric pressure (0 mmHg adjustment).

Subsequently, the CPU 100 operates as the pump drive unit 203 of the pressure control unit 201, closes the valve 33, and then drives the pump 32 via the pump drive circuit 320 to perform control to send air to the pressing cuff 21. As a result, the pressing cuff 21 is inflated and the cuff pressure Pc (see FIG. 9) is gradually increased to press the left wrist 90 as the measurement target site (step S3 in FIG. 8B).

In this pressing process, the CPU 100 operates as the pressure detection unit 202 of the pressure control unit 201 to calculate the blood pressure value, monitors the cuff pressure Pc by the pressure sensor 31, and acquires fluctuation component of the arterial volume generated in the radial artery 91 of the left wrist 90 as a pulse wave signal Pm as illustrated in FIG. 9.

Next, in step S4 in FIG. 8B, the CPU 100 operates as a second blood pressure calculation unit, and applies a known algorithm by an oscillometric method based on the pulse wave signal Pm acquired at this time to attempt to calculate blood pressure values (systolic blood pressure SBP and diastolic blood pressure DBP).

At this time, in a case where the blood pressure value cannot be calculated yet due to lack of data (NO in step S5), the processes in steps S3 to S5 are repeated unless the cuff pressure Pc reaches an upper limit pressure (for example, 300 mmHg is set in advance for safety).

In a case where the blood pressure value can be calculated in this manner (YES in step S5), the CPU 100 performs control to stop the pump 32, open the valve 33, and discharge the air in the pressing cuff 21 (step S6). Finally, the CPU 100 serves as the output unit 205, displays the measurement result of the blood pressure value on the display unit 50 and records the result in the memory 51 (step S7).

Note that the calculation of the blood pressure value is not limited to the pressing process, and may be performed in a decompression process.

(Operation for Blood Pressure Measurement based on Pulse Wave Transit Time)

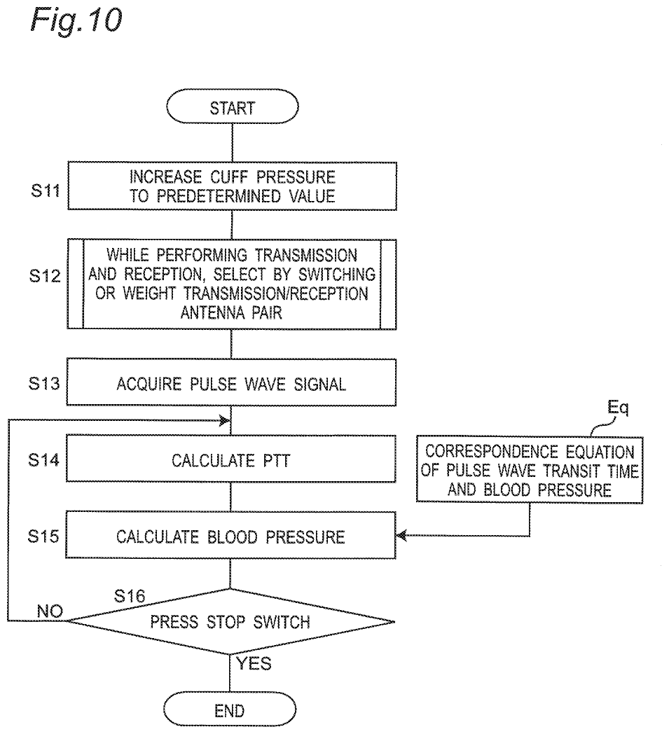

FIG. 10 is an operation flow according to the biological information measuring method, pulse wave measuring method, and blood pressure measuring method according to an embodiment of the present invention, in which the sphygmomanometer 1 performs pulse wave measurement to acquire the pulse wave transit time (PTT) and measure (estimate) blood pressure based on the pulse wave transit time. The belt 20 of the sphygmomanometer 1 is assumed to be worn in advance so as to surround the left wrist 90.

When the user instructs blood pressure measurement based on the PTT with a push-type switch as the operation unit 52 provided in the main body 10, the CPU 100 starts operation. In other words, the CPU 100 closes the valve 33, drives the pump 32 via the pump drive circuit 320, and performs control to send air to the pressing cuff 21, thereby expanding the pressing cuff21 and pressing the cuff pressure Pc (see FIG. 7A) to a predetermined value (step S11 in FIG. 10). In this example, in order to lighten the physical burden on the user, the pressure is kept high enough (for example, about 5 mmHg) just to make the belt 20 be in close contact with the left wrist 90. Thus, the transmission/reception antenna group 40E is securely brought into contact with the palmar side surface 90a of the left wrist 90, so that no gap is generated between the palmar side surface 90a and the transmission/reception antenna group 40E. Note that step S11 may be omitted.

At this time, as described with reference to FIG. 7A, with respect to the longitudinal direction of the left wrist 90 (corresponding to the width direction Y of the belt 20), the first set of transmission/reception antenna pairs (41, 42) is placed corresponding to the upstream portion 91u of the radial artery 91 passing through the left wrist 90 while the second set of transmission/reception antenna pairs (44, 43) is placed corresponding to the downstream portion 91d of the radial artery 91.

Next, in this wearing state, as described in step S12 of FIG. 10, the CPU 100 controls transmission and reception in each of the first pulse wave sensor 40-1 and the second pulse wave sensor 40-2 illustrated in FIG. 5.

More specifically, as illustrated in FIG. 7A, in the first pulse wave sensor 40-1, the transmission circuit 46 emits radio waves E1 toward the upstream portion 91u of the radial artery 91 via the transmission antenna array 41, that is, from the conductor layer 401 to the dielectric layer 402 (or an air gap existing around the side of the dielectric layer 402). At the same time, the reception circuit 47 receives, by the conductor layer 401, the radio waves E1′ reflected by the upstream portion 91u of the radial artery 91 via the reception antenna array 42, that is, via the dielectric layer 402 (or an air gap existing around the side of the dielectric layer 402), and detects and amplifies the radio waves E1′. Further, in the second pulse wave sensor 40-2, the transmission circuit 49 emits radio waves E2 toward the downstream portion 91d of the radial artery 91 via the transmission antenna array 44, that is, from the conductor layer 401 to the dielectric layer 402 (or an air gap existing around the side of the dielectric layer 402). At the same time, the reception circuit 48 receives, by the conductor layer 401, radio waves E2′ reflected by the downstream portion 91d of the radial artery 91 via the reception antenna array 43, that is, via the dielectric layer 402 (or an air gap existing around the side of the dielectric layer 402).

In step S12 of FIG. 10, while performing such transmission and reception, the CPU 100 serves as the antenna control units 111 and 112 to select by switching a transmission/reception antenna pair to be used from the transmission/reception antenna pairs (TX1, RX1), (TX2, RX2), (TX3, RX3), and (TX4, RX4) included in the first set of transmission/reception antenna pairs (41, 42) and select by switching a transmission/reception antenna pair to be used from the transmission/reception antenna pairs (TX1, RX1), (TX2, RX2), (TX3, RX3), and (TX4, RX4) included in the second set of transmission/reception antenna pairs (44, 43). This selection process in step S12 will be described in detail later.

Next, as described in step S13 of FIG. 10, the CPU 100 serves as the pulse wave detection units 101 and 102 in the respective first pulse wave sensor 40-1 and second pulse wave sensor 40-2 illustrated in FIG. 5 and acquires the pulse wave signals PSI and PS2 as illustrated in FIG. 7B. That is, in the first pulse wave sensor 40-1, the CPU 100 serves as the pulse wave detection unit 101, and acquires the pulse wave signal PS1 indicating the pulse wave of the upstream portion 91u of the radial artery 91 from an output during a vasodilation period and an output during a vasoconstriction period of the reception circuit 47, based on an output of the reception circuit 47 received via the transmission/reception antenna pair, which are selected or weighted in the first set of transmission/reception antenna pairs (41, 42). Further, in the second pulse wave sensor 40-2, the CPU 100 serves as the pulse wave detection unit 102 and acquires the pulse wave signal PS2 indicating the pulse wave of the downstream portion 91d of the radial artery 91 from an output during the vasodilation period and an output during the vasoconstriction period of the reception circuit 48, based on an output of the reception circuit 48 received via the transmission/reception antenna pair, which are selected or weighted in the second set of transmission/reception antenna pairs (44, 43).

Next, as illustrated in step S14 of FIG. 10, the CPU 100 serves as the PTT calculation unit 103 as a time difference acquisition unit, and calculates the time difference between the pulse wave signal PS1 and the pulse wave signal PS2 as the pulse wave transit time (PTT). More specifically, in this example, the time difference Δt between the peak A1 of the first pulse wave signal PS1 and the peak A2 of the second pulse wave signal PS2 illustrated in FIG. 7B is acquired as a pulse wave transit time (PTT).

Thereafter, as described in step S15 of FIG. 10, the CPU 100 serves as the first blood pressure calculation unit, and calculates (estimates) blood pressure based on the pulse wave transit time (PTT) acquired in step S14, using a predetermined correspondence equation Eq between the pulse wave transit time and the blood pressure. Here, the predetermined correspondence equation Eq between the pulse wave transit time and the blood pressure is provided as a known fractional function including a 1/DT² term as follows when the pulse wave transit time is represented as DT and the blood pressure is represented as EBP, for example:

EBP=α/DT²+β (Eq.1)

(Here, α and β each represent a known coefficient or constant.) (see JP 10-201724 A, for example). In addition, as the predetermined correspondence equation Eq between the pulse wave transit time and the blood pressure may be a different known correspondence equation including 1/DT term and a DT term in addition to the 1/DT² term as follows:

EBP=α/DT²+β/DT+γDT+δ (Eq.2)

(Here, α, β, γ, and δ each represent a known coefficient or constant.).

In this manner, the pulse wave signals PS1 and PS2 as biological information are acquired, the pulse wave transit time (PTT) is acquired, and the blood pressure value is calculated (estimated) based on the result. Note that the measurement result of the blood pressure value is displayed on the display unit 50 and recorded in the memory 51.

In this example, in a case where measurement stop is not instructed by the push-type switch as the operation unit 52 in step S16 of FIG. 10 (NO in step S16), the calculation of the pulse wave transit time (PTT) (step S14) and the calculation (estimation) of the blood pressure (step S15) are repeated periodically each time when the first and second pulse wave signals PS1 and PS2 are input according to the pulse wave. The CPU 100 updates and displays the blood pressure value measurement result on the display unit 50, and stores and records the result in the memory 51. Then, when measurement stop is instructed in step S16 of FIG. 10 (YES in step S16), the measurement operation is terminated.

With the sphygmomanometer 1, blood pressure can be continuously measured over a long period of time with a light physical burden on the user by measuring blood pressure based on the pulse wave transit time (PTT).

Further, according to the sphygmomanometer 1, the blood pressure measurement (estimation) based on the pulse wave transit time and the blood pressure measurement by the oscillometric method can be performed using the common belt 20 with a single device. This can improve the user convenience. For example, in general, when blood pressure measurement (estimation) based on the pulse wave transit time (PTT) is performed, calibration of the correspondence equation Eq between the pulse wave transit time and the blood pressure is appropriately performed (in the above example, update of values of coeflicients a and 3 based on the actually measured pulse wave transit time and the blood pressure value) needs to be performed. Here, according to the sphygmomanometer 1, the blood pressure measurement by the oscillometric method can be performed with the same apparatus, and the correspondence equation Eq can be calibrated based on the results, so that the convenience for the user is improved. In addition, a rapid increase in blood pressure can be captured by the PTT method (blood pressure measurement based on pulse wave transit time) that can be continuously measured, although accuracy is low, and the measurement with the more accurate oscillometric method can be started using the rapid increase in blood pressure as a trigger.

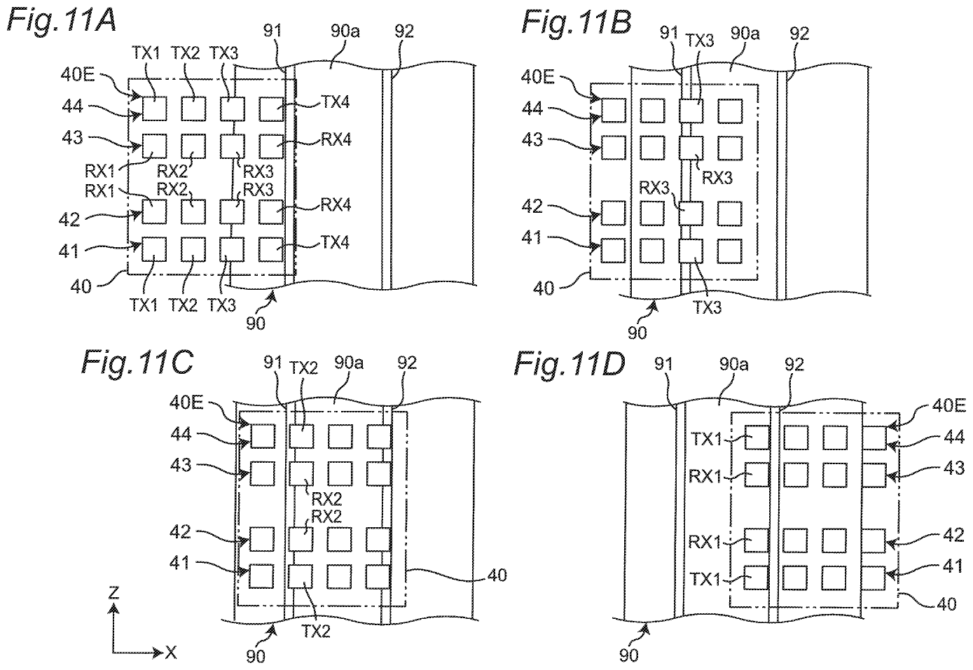

Here, in a case where measurement is performed in this manner, for example, as illustrated in FIGS. 11A to 11D, a position displacement of the transmission/reception antenna group 40E may occur with respect to the radial artery 91 in the longitudinal direction X of the belt every time the belt 20 is worn to the left wrist 90. For example, FIG. 11A illustrates a case where the transmission/reception antenna group 40E is largely displaced to the left with respect to the radial artery 91. FIG. 11B illustrates a case where the transmission/reception antenna group 40E is slightly displaced to the left with respect to the radial artery 91. FIG. 11C illustrates a case where the transmission/reception antenna group 40E is slightly displaced to the right with respect to the radial artery 91. FIG. 11D illustrates a case where the transmission/reception antenna group 40E is largely displaced to the right with respect to the radial artery 91. Note that, in the longitudinal direction X of the belt, it is assumed that there is no position displacement in a case where the radial artery 91 is between the transmission/reception antenna pairs (TX2, RX2) and (TX3, RX3) included in the first set of transmission/reception antenna pairs (41, 42), and between the transmission/reception antenna pairs (TX2, RX2) and (TX3, RX3) included in the second set of transmission/reception antenna pairs (44, 43).

### (Method for Selecting by Switching Transmission/Reception Antenna Pair)

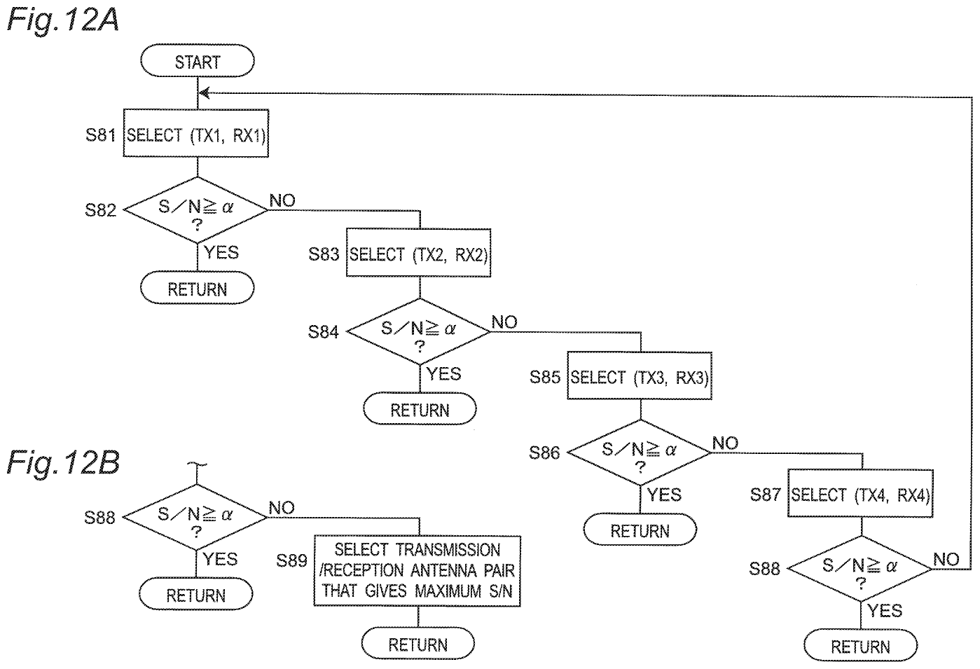

Therefore, in this sphygmomanometer 1, while performing transmission and reception in step S12 of FIG. 10 described above, the CPU 100 serves as the antenna control units 111 and 112, and performs control to select by switching the transmission/reception antenna pair as described in the operation flow of FIG. 12A. In the following description, it is assumed that when an antenna element is not explicitly described as “selected”, the antenna element is not selected.

First, as described in step S81 of FIG. 12A, in this example, the transmission/reception antenna pair (TX1, RX1) located at the left end is selected from the transmission/reception antenna pairs (TX1, RX1), (TX2, RX2) (TX3, RX3), and (TX4, RX4) included in the first set of transmission/reception antenna pairs (41, 42), and the transmission/reception antenna pair (TX1, RX1) located at the left end is selected from the transmission/reception antenna pair (TX1, RX1), (TX2, RX2), (TX3, RX3), and (TX4, RX4) included in the second set of transmission/reception antenna pairs (44, 43) (corresponding to later described “first time” in Table 1). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S82 of FIG. 12A, the CPU 100 serves as the antenna control units 111 and 112, acquires a signal-to-noise ratio (S/N) of the pulse wave signals PS1 and PS2, and determines whether or not the acquired S/Ns are both larger than a threshold value α as a reference value (in this example, it is defined as α=40 dB in advance. The same applies hereinafter.). Here, in a case where both S/N are equal to or larger than α (YES in step S82), it is determined that the current transmission/reception antenna pair selection is appropriate, and the process returns to the main flow (FIG. 10). For example, in a case where the transmission/reception antenna group 40E is largely displaced to the right with respect to the radial artery 91 as illustrated in FIG. 11D, this may correspond to the above case.

On the other hand, in a case where S/N in either of the pulse wave signals PS1 and PS2 is smaller than α in step S82 of FIG. 12A (NO in step S82), the process proceeds to step S83, and the CPU 100 serves as the antenna control units 111 and 112 to select the transmission/reception antenna pair (TX2, RX2) located on the right side of (TX1, RX1) from the transmission/reception antenna pairs (TX1, RX1), (TX2, RX2), (TX3, RX3), and (TX4, RX4) included in the first set of transmission/reception antenna pairs (41, 42) and select the transmission/reception antenna pair (TX2, RX2) located on the right side of (TX1, RX1) from the transmission/reception antenna pairs (TX1, RX1), (TX2, RX2), (TX3, RX3), and (TX4, RX4), included in the second set of transmission/reception antenna pairs (44, 43) (equivalent to “second time” in Table 1 below). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S84 of FIG. 12A, the CPU 100 serves as the antenna control units 111 and 112, acquires the signal-to-noise ratios (S/N) of the pulse wave signals PS1 and PS2, and determines whether or not the acquired S/Ns are larger than the threshold value α. Here, in a case where both S/Ns are equal to or larger than α (YES in step S84), it is determined that the current transmission/reception antenna pair selection is appropriate, and the process returns to the main flow (FIG. 10). For example, a case where the transmission/reception antenna group 40E is slightly displaced to the right with respect to the radial artery 91 as illustrated in FIG. 11C may correspond to the above case.

On the other hand, in a case where S/Ns in either the pulse wave signals PS1 and PS2 are smaller than α in step S84 of FIG. 12A (NO in step S84), the process proceeds to step S85, and the CPU 100 serves as the antenna control units 111 and 112 to select the transmission/reception antenna pair (TX3, RX3) located on the right side of (TX2, RX2) from the transmission/reception antenna pairs (TX1, RX1), (TX2, RX2), (TX3, RX3), and (TX4, RX4) included in the first set of transmission/reception antenna pairs (41, 42), and to select the transmission/reception antenna pair (TX3, RX3) located on the right side of (TX2, RX2) from the transmission/reception antenna pair (TX1, RX1), (TX2, RX2), (TX3, RX3), and (TX4, RX4) included in the second set of transmission/reception antenna pairs (44, 43) (equivalent to “third time” in Table 1 below). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S86 of FIG. 12A, the CPU 100 serves as the antenna control units 111 and 112, acquires the signal-to-noise ratios (S/N) of the pulse wave signals PS1 and PS2, and determines whether or not the acquired S/Ns are both larger than the threshold value α. Here, in a case where the both S/Ns are equal to or larger than α (YES in step S86), it is determined that the current transmission/reception antenna pair selection is appropriate, and the process returns to the main flow (FIG. 10). For example, a case where the transmission/reception antenna group 40E is slightly displaced to the left with respect to the radial artery 91 as illustrated in FIG. 1B, this may correspond to the above case.

On the other hand, in a case where S/Ns in either of the pulse wave signals PS1 and PS2 are smaller than α in step S86 of FIG. 12A (NO in step S86), the process proceeds to step S87, and the CPU 100 serves as the antenna control units 111 and 112 to select the transmission/reception antenna pair (TX4, RX4) located on the right side (right end) of (TX3, RX3) from the transmission/reception antenna pairs (TX1, RX1), (TX2, RX2), (TX3, RX3), and (TX4, RX4) included in the first set of transmission/reception antenna pairs (41, 42) and select the transmission/reception antenna pair (TX4, RX4) located on the right side (right end) of (TX3, RX3) from the transmission/reception antenna pairs (TX1, RX1), (TX2, RX2), (TX3, RX3), and (TX4, RX4) included in the second set of transmission/reception antenna pairs (44, 43) (relevant to “fourth time” in Table 1 below). In response to this selection, the CPU 100 serves as the pulse wave detection units 101 and 102 to acquire pulse wave signals PSI and PS2 indicating the pulse waves of the upstream portion 91u and the downstream portion 91d of the radial artery 91 described above.

Next, as described in step S88 of FIG. 12A, the CPU 100 serves as the antenna control units 111 and 112, acquires the signal-to-noise ratios (S/N) of the pulse wave signals PS1 and PS2, and determines whether or not the acquired S/Ns are larger than the threshold value α. Here, in a case where the both S/Ns are equal to or larger than α (YES in step S88), it is determined that the selection of the current transmission/reception antenna pair is appropriate, and the process returns to the main flow (FIG. 10). For example, as described in FIG. 11A, the case where the transmission/reception antenna group 40E is largely displaced to the left with respect to the radial artery 91 may correspond to the above case.

On the other hand, in a case where S/Ns in either of the pulse wave signals PS1 and PS2 are smaller than α in step S88 of FIG. 12A (NO in step S88), the process returns to step S81 and the processing is repeated. Note that, in a case where a transmission/reception antenna pair suitable for use is not found even when the processing of steps S81 to S88 in FIG. 12A is repeated a predetermined number of times, or a case where a transmitted/received antenna pair suitable for use is not found even after a predetermined period has elapsed, the CPU 100 displays an error on the display unit 50 and ends the process, in this example.

TABLE 1

| | TX1 | TX2 | TX3 | TX4 |
|---|---|---|---|---|
| Number of Times | RX1 | RX2 | RX3 | RX4 |
| First time | Select | — | — | — |
| Second time | — | Select | — | — |
| Third time | — | — | Select | — |
| Fourth time | — | — | — | Select |

(In Table 1, the symbol “—” indicates “not selected.” The same applies to the following tables.)

As described above, in the operation flow of FIG. 12A, for the first set of transmission/reception antenna pairs (41, 42) and the second set of transmission/reception antenna pairs (44, 43), the CPU 100 selects by switching from the transmission/reception antenna pair (TX1, RX1) arranged at the end on one side (the left side in this example) with respect to the longitudinal direction X of the belt 20 and then sequentially to the transmitter/receiver antenna pair (TX4, RX4) arranged at the other side (the right side in this example) as described in the above Table 1 respectively, to search for a transmitter/receiver antenna pair having a large signal-to-noise ratio (S/N). Thereby, a transmission/reception antenna pair suitable for use can be reliably determined among the plurality of transmission/reception antenna pairs (TX1, RX1), (TX2, RX2), (TX3, RX3), and (TX4, RX4). Therefore, the signal-to-noise ratio (S/N) of the received signal can be increased, and as a result, the pulse wave signal, pulse wave transit time, and blood pressure as biological information can be accurately measured.

Further, in the first set of transmission/reception antenna pairs (41, 42) and the second set of transmission/reception antenna pairs (44, 43), during a process for selecting by switching the respective transmission/reception antenna pairs (TX1, RX1), (TX2, RX2), (TX3, RX3), and (TX4, RX4), the switching can be stopped and the process can be completed when an acquired signal-to-noise ratio (S/N) is larger than the threshold value α. Therefore, the selection process can be completed more quickly than α case where all the switching operations are performed.

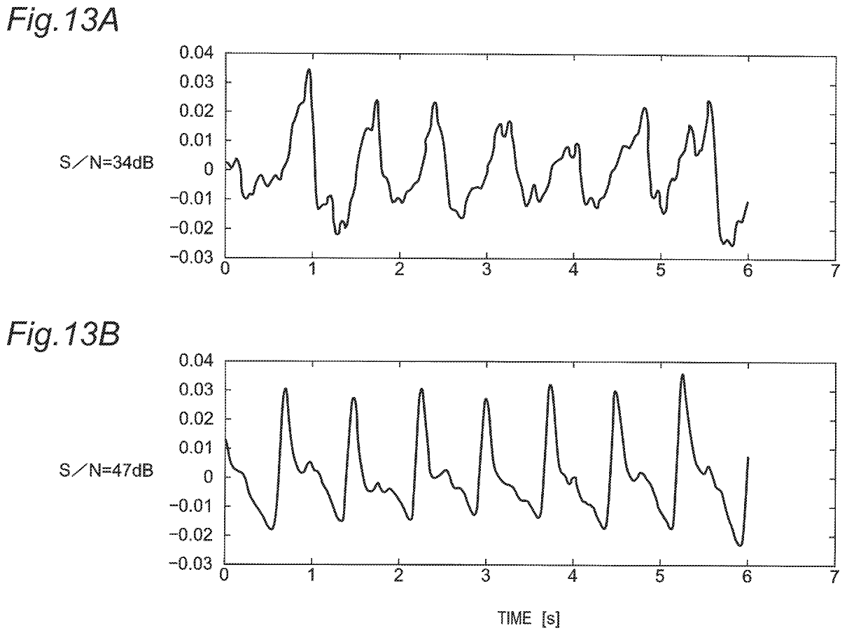

FIG. 13A illustrates the waveforms of the pulse wave signals PS1 and PS2 acquired as a result of the position displacement of the transmission/reception antenna group 40E with respect to the radial artery 91 in the longitudinal direction X of the belt. In this example, the S/N of the pulse wave signals PS1 and PS2 is 34 dB. On the other hand, FIG. 13B illustrates the waveforms of the pulse wave signals PS1 and PS2 acquired by the operation flow of FIG. 12A. In this example, the S/N of the pulse wave signals PS1 and PS2 is 47 dB. Thus, the signal-to-noise ratio (S/N) of the received signals (pulse wave signals PS1 and PS2 in this example) can be increased.

Here, in the above example, in a case where a transmission/reception antenna pair suitable for use is not found even after repeating the processing of steps S81 to S88 in FIG. 12A a predetermined number of times, or in a case where a transmission/reception antennas suitable for use cannot be found even after a predetermined period has elapsed, the CPU 100 displays an error on the display unit 50 and ends the processing. However, this example does not set any limitation. For example, it is assumed that the CPU 100 stores the signal-to-noise ratio (S/N) of the pulse wave signals PS1 and PS2 in the memory 51 in steps S82, S84, S86, and S88 in FIG. 12A. Then, in a case of NO in step S88 of FIG. 12A, as described in step S89 of FIG. 12B, a transmission/reception antenna pair that gives the maximum S/N may be selected from the plurality of transmission/reception antenna pairs (TX1, RX1), (TX2, RX2), (TX3, RX3), and (TX4, RX4).

Further, as a matter of course, as described in Table 2 below, in each of the first set of transmission/reception antenna pairs (41, 42) and the second set of transmission/reception antenna pairs (44, 43), the CPU 100 can sequentially select by switching from the transmission/reception antenna pair (TX4, RX4) arranged at the right end with respect to longitudinal direction X of the belt 20 to the transmission/reception antenna pair (TX1, RX1) arranged at the left end to search for a transmission/reception antenna pair with which the signal-to-noise ratio (S/N) becomes larger. Even in this case, it is possible to reliably determine a transmission/reception antenna pair which is suitable for use from a plurality of transmission/reception antenna pairs (TX1, RX1), (TX2, RX2), (TX3, RX3), and (TX4, RX4).

TABLE 2

| | TX1 | TX2 | TX3 | TX4 |
|---|---|---|---|---|
| Number of Times | RX1 | RX2 | RX3 | RX4 |
| First time | — | — | — | Select |
| Second time | — | — | Select | — |
| Third time | — | Select | — | — |
| Fourth time | Select | — | — | — |

Further, when the belt 20 is worn to the left wrist 90, the amount of positional displacement of the transmission/reception antenna group 40E with respect to the left wrist 90 is assumed to indicate frequency of normal distribution centered on an area corresponding to the radial artery 91 in the circumferential direction of the left wrist 90 from a statistical viewpoint. Therefore, in the first set of transmission/reception antenna pairs (41, 42) and the second set of transmission/reception antenna pairs (44, 43), respectively, the CPU 100 may sequentially select by switching from the transmission/reception antenna pair (TX2, RX2) arranged at an almost center in the longitudinal direction X of the belt 20, as described in Table 3 below, to the antenna elements arranged at ends in opposite sides alternately to search for a transmission/reception antenna pair with which the signal-to-noise ratio (S/N) becomes larger. This makes it possible to reliably and quickly determine a transmission/reception antenna pair suitable for use from the plurality of transmission/reception antenna pairs (TX1, RX1), (TX2, RX2), (TX3, RX3), and (TX4, RX4).

TABLE 3

| | TX1 | TX2 | TX3 | TX4 |
|---|---|---|---|---|
| Number of Times | RX1 | RX2 | RX3 | RX4 |
| First time | — | Select | — | — |
| Second time | — | — | Select | — |
| Third time | Select | — | — | — |
| Fourth time | — | — | — | Select |

Further, in this example, the left and right in Table 3 may be exchanged as described in Table 4 below, and the CPU 100 may sequentially select by switching from the transmission/reception antenna pair (TX3, RX3) arranged at almost center in the longitudinal direction X of the belt 20 to the antenna elements arranged at the ends in opposite sides alternately to search for a transmission/reception antenna pair with which the signal-to-noise ratio (S/N) becomes larger. In this case as well, it is possible to reliably and quickly determine a suitable transmission/reception antenna pair from the plurality of transmission/reception antenna pairs (TX1, RX1), (TX2, RX2), (TX3, RX3), and (TX4, RX4).

TABLE 4

| | TX1 | TX2 | TX3 | TX4 |
|---|---|---|---|---|
| Number of Times | RX1 | RX2 | RX3 | RX4 |
| First time | — | — | Select | — |
| Second time | — | Select | — | — |
| Third time | — | — | — | Select |
| Fourth time | Select | — | — | — |

In the above examples, in the first set of transmission/reception antenna pairs (41, 42) and the second set of transmission/reception antenna pairs (44, 43), a transmission/reception antenna pair lined up along the width direction Y of the belt 20 and having the same numbers are selected in conjunction with each other. However, this example does not set any limitation. The selection of the transmission/reception antenna pair in the first set of transmission/reception antenna pairs (41, 42) and the selection of the transmission/reception antenna pair in the second set of transmission/reception antenna pairs (44, 43) may be performed independently from each other. With this configuration, in a case where the belt 20 is worn to the left wrist 90, and the belt 20 obliquely intersects the radial artery 91 so that the transmission/reception antenna group 40E is obliquely displaced in the paper plane of FIG. 3 for example, a transmission/reception antenna pairs suitable for use can be selected respectively in the first set of transmission/reception antenna pairs (41, 42) and the second set of transmission/reception antenna pairs (44, 43). Therefore, the signal-to-noise ratio (S/N) of the received signal can be increased, and as a result, the pulse wave signal, pulse wave transit time, and blood pressure as biological information can be accurately measured.

### (Method for Weighting Transmission/Reception Antenna Pairs)

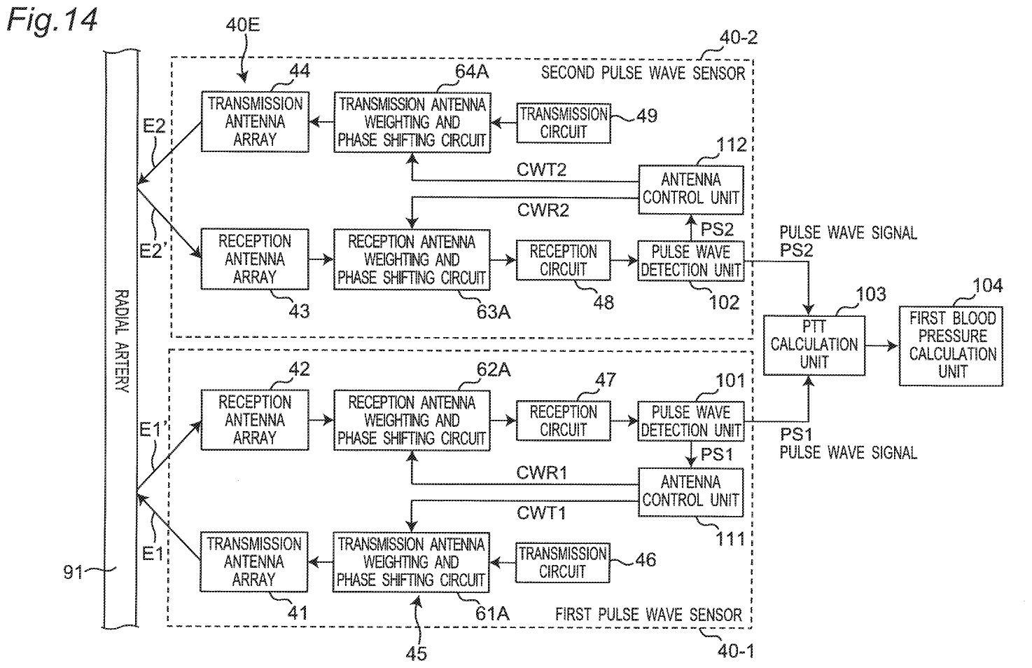

FIG. 14 illustrates an example that the sphygmomanometer 1 includes transmission antenna weighting and phase shift circuits 61A and 64A, reception antenna weighting and phase shift circuits 62A and 63A as a substitute for the transmission antenna switching circuits 61 and 64 and the reception antenna switching circuits 62 and 63 illustrated in FIG. 5. These transmission antenna weighting and phase shift circuits 61A and 64A and the reception antenna weighting and phase shift circuits 62A and 63A may be realized by hardware such as a switching element, or may be realized by software by a program in the CPU 100.

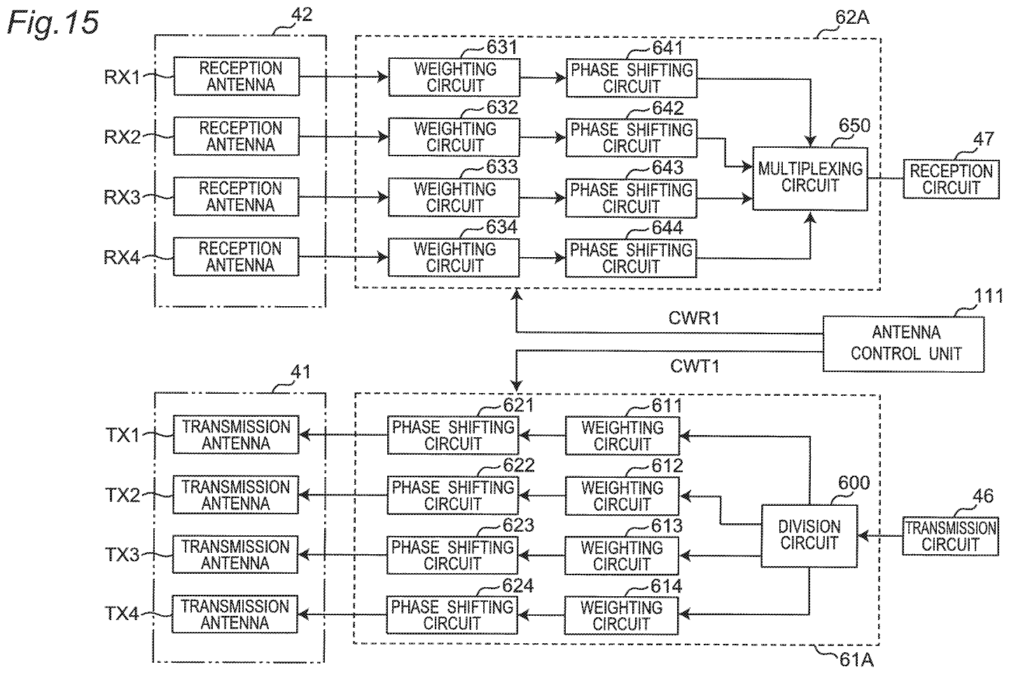

In this example, as illustrated in FIG. 15, the transmission antenna weighting and phase shift circuit 61A includes a division circuit 600 that evenly divides signal from the transmission circuit 46 into four according to the transmission antennas TX1, TX2, TX3, and TX4 included in the transmission antenna array 41, and weighting circuits 611, 612, 613, and 614 respectively provided corresponding to the transmission antennas TX1, TX2, TX3, and TX4, and a phase shift circuits 621, 622, 623, and 624 respectively provided corresponding to the transmission antennas TX1, TX2, TX3, and TX4. The weighting circuits 611, 612, 613, and 614 multiplex amplitude of the signal received from the division circuit 600 into m1, m2, m3, and m4 times respectively (in this example, it is assumed as 0≤m1, m2, m3, m4≤1) according to the transmission antenna control signal CWT1 from the antenna control unit 111. With this configuration, weights m1, m2, m3, and m4 are assigned to the transmission antennas TX1, TX2, TX3, and TX4, respectively. The phase shift circuits 621, 622, 623, and 624 shift the phases of the signals received from weighting circuits 611, 612, 613, and 614, respectively, according to transmission antenna control signal CWT1 from antenna control unit 111. With this configuration, the phases of the radio waves emitted via the transmission antennas TX1, TX2, TX3, and TX4 are shifted relative to each other.

The reception antenna weighting and phase shift circuit 62A includes weighting circuits 631, 632, 633, and 634 provided respectively corresponding to reception antennas RX1, RX2, RX3, and RX4 included in the reception antenna array 42, phase shift circuits 641, 642, 643, and 644 provided respectively corresponding to reception antennas RX1, RX2, RX3, and RX4, and a multiplexing circuit 650 for multiplexing signals received by the reception antennas RX1, RX2, RX3, and RX4 (outputs of the phase shift circuits 641, 642, 643, and 644). The weighting circuits 631, 632, 633, and 634 multiplexes the amplitudes of the signals received through the reception antennas RX1, RX2, RX3, and RX4 to n1, x2, n3, and n4 times respectively (in this example, 0≤n1, n2, n3, n4≤1) according to the reception antenna control signal CWR1 from the antenna control unit Ill. With this configuration, weights n1, n2, n3, and n4 are assigned to the reception antennas RX1, RX2, RX3, and RX4, respectively. The phase shift circuits 641, 642, 643, and 644 shift the phases of the signals received from weighting circuits 631, 632, 633, and 634, respectively, according to the reception antenna control signal CWR1 from antenna control unit 111. With this configuration, the phases of the signals received via the reception antennas RX1, RX2, RX3, and RX4 are shifted relative to each other.

Further, the transmission antenna weighting and phase shift circuit 64A illustrated in FIG. 14 is configured in the similar manner as the transmission antenna weighting and phase shift circuit 61A, weights the transmission antennas TX1, TX2, TX3 and TX4 respectively with m1′, m2′, m3′, and m4′ (in this example, it is assumed as 0≤m1′, m2′, m3′, m4′≤1) and shifts the phases of radio waves emitted via the transmission antennas TX1, TX2, TX3, and TX4 included in the transmission antenna array 44 relative to each other, according to the transmission antenna control signal CWT2 from the antenna control unit 111. Further, the reception antenna weighting and phase shift circuit 63A is configured in the similar manner as the reception antenna weighting and phase shift circuit 62A, weights the reception antenna RX1, RX2, RX3, and RX4 included in the reception antenna array 43 respectively with n1′, n2′, n3′, and n4′ (in this example, it is assumed as 0≤n1′, n2′, n3′, n4′≤1), and shifts the phases of signals received via the reception antennas RX1, RX2, RX3, and RX4 relative to each other, according to the reception antenna control signal CWR2 from the antenna control unit 111.

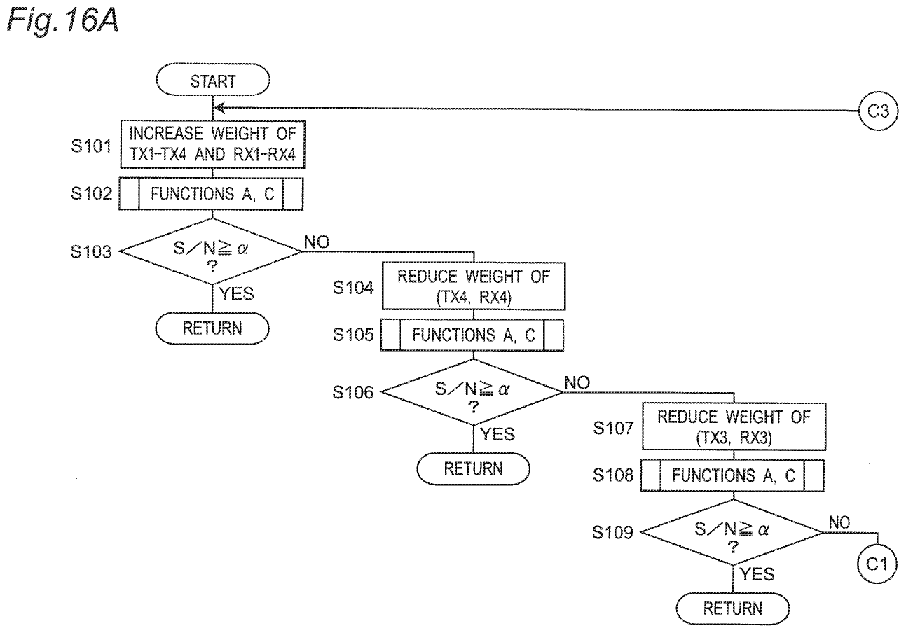

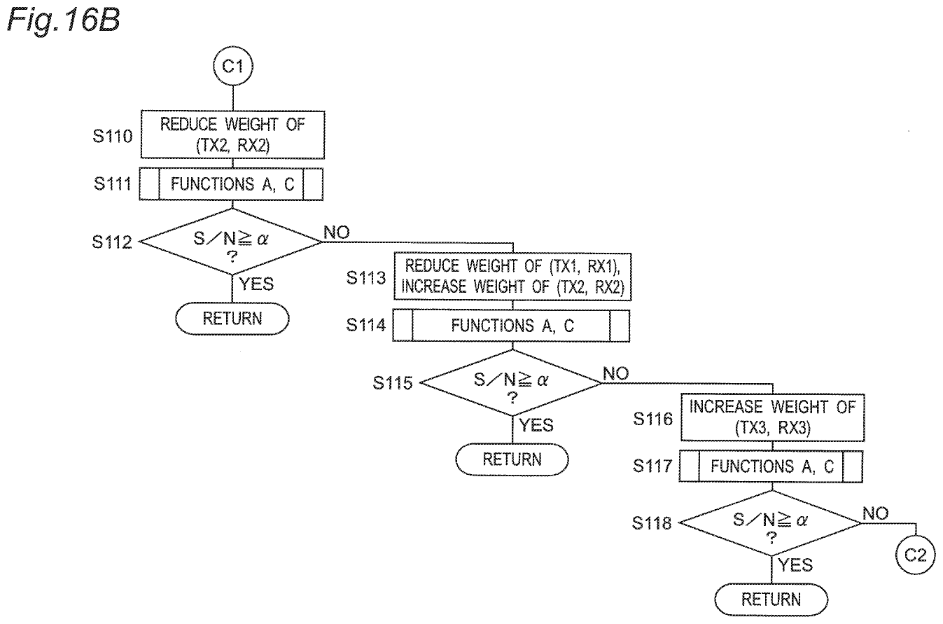

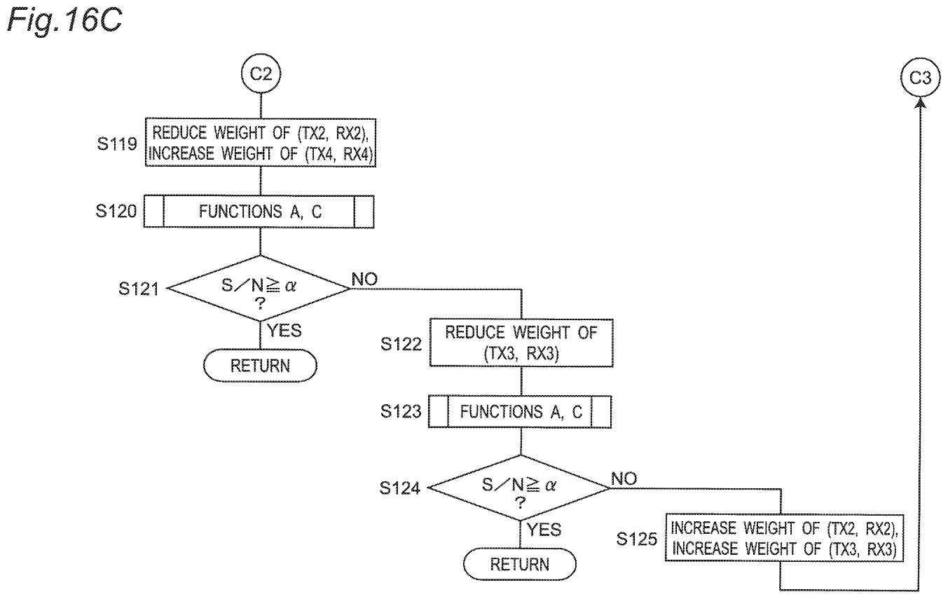

In this example, basically the same operation flow illustrated in FIG. 10 is executed for blood pressure measurement based on the pulse wave transit time. Then, in step S12 in FIG. 10, while performing the above-described transmission and reception, the CPU 100 serves as the antenna control units 111 and 112, and controls to weight the transmission/reception antenna pairs (TX1, RX1), (TX2, RX2), (TX3, RX3), and (TX4, RX4) included in the first set of transmission/reception antenna pairs (41, 42) and the transmission/reception antenna pairs (TX1, RX1), (TX2, RX2), (TX3, RX3), and (TX4, RX4) included in the second set of transmission/reception antenna pairs (44, 43) as illustrated in FIGS. 16A to 16C.

Note that, in the examples of FIGS. 16A to 16C, for the sake of simplicity, in the first set of transmission/reception antenna pairs (41, 42) and the second set of transmission/reception antenna pairs (44, 43), the weights of the transmission antennas TX1, TX2, TX3, and TX4 and the weights of the reception antennas RX1, RX2, RX3, and RX4 are switched into a large level (in this example, weight 1) or small level (in this example, weight 0.1) in conjunction with each other.

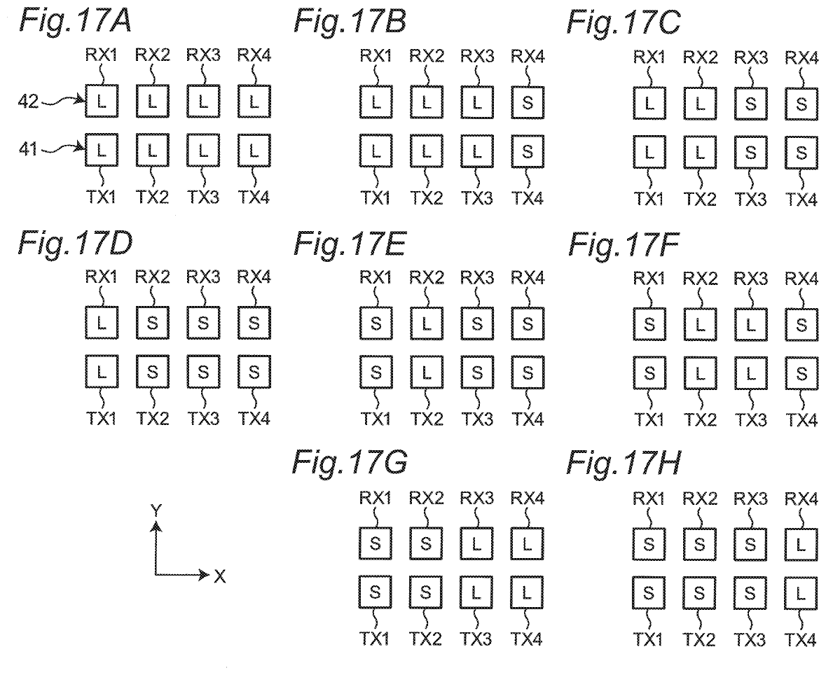

More specifically, first, as described in step S101 of FIG. 16A, the CPU 100 serves as the antenna control units 111 and 112, and sets the weights of the transmission/reception antenna pairs (TX1, RX1), (TX2, RX2), (TX3, RX3), and (TX4, RX4) in the large level respectively in the first set of transmission/reception antenna pairs (41, 42) and the second set of transmission/reception antenna pairs (44, 43). For example, as schematically illustrated in FIG. 17A, in the first set of transmission/reception antenna pairs (41, 42), the weights of the transmission antennas TX1, TX2, TX3, and TX4 and the reception antennas RX1, RX2, RX3, and RX4 are all in the large level. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S102 of FIG. 16A, the CPU 100 serves as the antenna control units 111 and 112, and controls to shift the relative relative phase of the radio waves emitted by the transmission antennas TX1, TX2, TX3, and TX4 and the relative phase of the signals received by the reception antennas RX1, RX2, RX3, and RX4, respectively in the first set of transmission/reception antenna pairs (41, 42) and second set of transmission/reception antenna pairs (44, 43), and control to increase the signal-to-noise ratio (S/N) of a combined signal obtained by combining the signals (which is referred to as “control of function A”). Further, the CPU 100 serves as the antenna control units 111 and 112 to change the relative weight of the radio waves emitted from the transmission antennas TX1, TX2, TX3, and TX4 and the relative weight of signals received by the reception antennas RX1, RX2, RX3, and RX4 in the first set of transmission/reception antenna pairs (41, 42) and the second set of transmission/reception antenna pairs (44, 43) respectively, and control to increase the signal-to-noise ratio (S/N) of the combined signal obtained by combining the signals (which is referred to as “control of function C”). The control of these functions A and C will be described in detail later.

Next, as described in step S103 of FIG. 16A, the CPU 100 serves as the antenna control units 111 and 112 to acquire the signal-to-noise ratio (S/N) of the pulse wave signals PS1 and PS2, and determines whether or not the acquired S/Ns are all larger than the threshold values a as a reference value (in this example, α=40 dB is defined in advance. The same applies below.). Here, in a case where any of the S/Ns are equal to or larger than α (YES in step S103), it is determined that the current weighting of the transmission/reception antenna pair is appropriate, and the process returns to the main flow (FIG. 10).

On the other hand, in step S103 in FIG. 16A, in a case where any of the S/Ns of the pulse wave signals PS1 and PS2 is smaller than α (NO in step S103), the process proceeds to step S104, and the CPU 100 serves as the antenna control units 111 and 112, and switches to set the weights of the transmission/reception antenna pair (TX4, RX4) to be the small level in the first set of transmission/reception antenna pairs (41, 42) and the second set of transmission/reception antenna pairs (44, 43). Thereby, as schematically illustrated in FIG. 17B, in the first set of transmission/reception antenna pairs (41, 42), the weights of the transmission antennas TX1, TX2, TX3 and the reception antennas RX1, RX2, RX3 are large, the weights of the transmission antenna TX4 and the reception antenna RX4 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S105 in FIG. 16A, the CPU 100 serves as the antenna control units 111 and 112 to control the above-described function A and function C.

Next, as described in step S106, the CPU 100 serves as the antenna control units 111 and 112 to acquire the signal-to-noise ratio (S/N) of the pulse wave signals PSI and PS2 and determine whether or not the acquired S/Ns are both larger than the threshold value α. Here, in a case where S/Ns are equal to or larger than α (YES in step S106), it is determined that the current weighting of the transmission/reception antenna pair is appropriate, and the process returns to the main flow (FIG. 10).

On the other hand, in a case where any of S/Ns of the pulse wave signals PS1 and PS2 is smaller than α in step S106 of FIG. 16A (NO in step S106), the process proceeds to step S107, and the CPU 100 serves as the antenna control units 111 and 112, to switch to set the weights of the transmission/reception antenna pair (TX3, RX3) to be the small level in the respective first set of transmission/reception antenna pairs (41, 42) and second set of transmission/reception antenna pairs (44, 43). With this configuration, as schematically illustrated in FIG. 17C, in the first set of transmission/reception antenna pairs (41, 42), the weights of the transmission antennas TX1 and TX2 and reception antennas RX1 and RX2 are large, and the the weights of the transmission antennas TX3 and TX4 and reception antennas RX3 and RX4 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S108 in FIG. 16A, the CPU 100 serves as the antenna control units 111 and 112 to control the above-described function A and function C.

Next, as described in step S109, the CPU 100 serves as the antenna control units 111 and 112 to acquire the signal-to-noise ratios (S/Ns) of the pulse wave signals PSI and PS2, and determine whether or not the acquired S/Ns are both larger than the threshold value α. Here, in a case where any of the S/Ns are equal to or larger than α (YES in step S109), it is determined that the current weighting of the transmission/reception antenna pair is appropriate, and the process returns to the main flow (FIG. 10). For example, a case where the transmission/reception antenna group 40E is slightly displaced to the right with respect to the radial artery 91 as illustrated in FIG. 11C may correspond to the above case.

On the other hand, in step S109 in FIG. 16A, in a case where any of the S/N of the pulse wave signals PS1 and PS2 is smaller than α (NO in step S109), the process proceeds to step S110 of FIG. 16B, and the CPU 100 serves as the antenna control units 111 and 112, and switches to set the weights of the transmission/reception antenna pair (TX2, RX2) to be the small level in the first set of transmission/reception antenna pairs (41, 42) and the second set of transmission/reception antenna pairs (44, 43). With this configuration, as schematically illustrated in FIG. 17D, in the first set of transmission/reception antenna pairs (41, 42), the weights of the transmission antenna TX1 and reception antenna RX1 are large, and the weights of the transmission antennas TX2, TX3 and TX4 and reception antennas RX2, RX3 and RX4 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S111 in FIG. 16B, the CPU 100 serves as the antenna control units 111 and 112 to control the above-described function A and function C.

Next, as described in step S112, the CPU 100 serves as the antenna control units 111 and 112 to acquire the signal-to-noise ratios (S/Ns) of the pulse wave signals PSI and PS2, and determine whether or not the acquired S/Ns are both larger than the threshold value α. Here, in a case where any of the S/Ns are equal to or larger than α (YES in step S112), it is determined that the current weighting of the transmission/reception antenna pair is appropriate, and the process returns to the main flow (FIG. 10). For example, in a case where the transmission/reception antenna group 40E is largely displaced to the right with respect to the radial artery 91 as illustrated in FIG. 11D, this may correspond to the above case.

On the other hand, in step S112 in FIG. 16B, in a case where any of the S/N of the pulse wave signals PS1 and PS2 is smaller than α (NO in step S112), the process proceeds to step S113, and the CPU 100 serves as the antenna control units 111 and 112, and switches to set the weight of the transmission/reception antenna pair (TX1, RX1) to be small and switches to set the weight of the transmission/reception antenna pair (TX2, RX2) to be large in the respective first set of transmission/reception antenna pairs (41, 42) and second set of transmission/reception antenna pairs (44, 43). With this configuration, as schematically illustrated in FIG. 17E, in the first set of transmission/reception antenna pairs (41, 42), the weights of the transmission antenna TX2 and reception antenna RX2 are large, and the weights of the transmission antennas TX1, TX3 and TX4 and reception antennas RX1, RX3 and RX4 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S114 in FIG. 16B, the CPU 100 serves as the antenna control units 111 and 112 to control the above-described function A and function C.

Next, as described in step S115, the CPU 100 serves as the antenna control units 111 and 112 to acquire the signal-to-noise ratios (S/Ns) of the pulse wave signals PS1 and PS2, and determine whether or not the acquired S/Ns are both larger than the threshold value α. Here, in a case where any of the S/Ns are equal to or larger than α (YES in step S115), it is determined that the current weighting of the transmission/reception antenna pair is appropriate, and the process returns to the main flow (FIG. 10).

On the other hand, in step S115 in FIG. 16B, in a case where any of the S/Ns of the pulse wave signals PS1 and PS2 is smaller than α (NO in step S115), the process proceeds to step S116, and the CPU 100 serves as the antenna control units 111 and 112, and switches to set the weights of the transmission/reception antenna pair (TX3, RX3) to be large in the first set of transmission/reception antenna pairs (41, 42) and the second set of transmission/reception antenna pairs (44, 43). With this configuration, as schematically illustrated in FIG. 17F, in the first set of transmission/reception antenna pairs (41, 42), the weights of the transmission antennas TX2 and TX3 and reception antennas RX2 and RX3 are large, and the weights of the transmission antennas TX1 and TX4 and reception antennas RX1 and RX4 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S117 in FIG. 16B, the CPU 100 serves as the antenna control units 111 and 112 to control the above-described function A and function C.

Next, as described in step S118, the CPU 100 serves as the antenna control units 111 and 112 to acquire the signal-to-noise ratios (S/Ns) of the pulse wave signals PSI and PS2, and determine whether or not the acquired S/Ns are both larger than the threshold value α. Here, in a case where any of the S/Ns are equal to or larger than α (YES in step S118), it is determined that the current weighting of the transmission/reception antenna pair is appropriate, and the process returns to the main flow (FIG. 10). For example, a case where the transmission/reception antenna group 40E is slightly displaced to the left with respect to the radial artery 91 as illustrated in FIG. 11B, this may correspond to the above case.

On the other hand, in step S118 in FIG. 16B, in a case where any of the S/Ns of the pulse wave signals PS1 and PS2 is smaller than α (NO in step S118), the process proceeds to step S119 in FIG. 16C, and the CPU 100 serves as the antenna control units 111 and 112, and switches to set the weight of the transmission/reception antenna pair (TX2, RX2) to be small and the weight of the transmission/reception antenna pair (TX4 RX4) to be large in the respective first set of transmission/reception antenna pairs (41, 42) and second set of transmission/reception antenna pairs (44, 43). Thereby, as schematically illustrated in FIG. 17G, in the first set of transmission/reception antenna pairs (41, 42), the weights of the transmission antennas TX3 and TX4 and reception antennas RX3 and RX4 are large, and the weights of the transmission antennas TX1 and TX2 and reception antennas RX1 and RX2 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S120 in FIG. 16C, the CPU 100 serves as the antenna control units 111 and 112 to control the above-described function A and function C.

Next, as described in step S121, the CPU 100 serves as the antenna control units 111 and 112 to acquire the signal-to-noise ratio (S/N) of the pulse wave signals PSI and PS2, and determine whether or not these acquired S/Ns are both larger than the threshold value α. Here, in a case where any of the S/Ns are equal to or larger than α (YES in step S121), it is determined that the current weighting of the transmission/reception antenna pair is appropriate, and the process returns to the main flow (FIG. 10).

On the other hand, in step S121 in FIG. 16C, in a case where any of the S/Ns of the pulse wave signals PS1 and PS2 is smaller than α (NO in step S121), the process proceeds to step S122, and the CPU 100 serves as the antenna control units 111 and 112, and switches to set the weights of the transmission/reception antenna pair (TX3, RX3) to be the small level in the first set of transmission/reception antenna pairs (41, 42) and the second set of transmission/reception antenna pairs (44, 43). Thereby, as schematically illustrated in FIG. 17H, in the first set of transmission/reception antenna pairs (41, 42), the weights of the transmission antenna TX4 and reception antenna RX4 are large, and the weights of the transmission antennas TX1, TX2, and TX3 and reception antennas RX1, RX2, and RX3 are small. The same applies to the second set of transmission/reception antenna pairs (44, 43). In accordance with this weighting, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S123 in FIG. 16C, the CPU 100 serves as the antenna control units 111 and 112 to control the above-described function A and function C.

Next, as described in step S124, the CPU 100 serves as the antenna control units 111 and 112 to acquire the signal-to-noise ratio (S/N) of the pulse wave signals PSI and PS2, and determine whether or not these acquired S/Ns are both larger than the threshold value α. Here, in a case where any of the S/Ns are equal to or larger than α (YES in step S124), it is determined that the current weighting of the transmission/reception antenna pair is appropriate, and the process returns to the main flow (FIG. 10). For example, as described in FIG. 11A, the case where the transmission/reception antenna group 40E is largely displaced to the left with respect to the radial artery 91 may correspond to the above case.

On the other hand, in step S124 in FIG. 16C, in a case where any of the S/Ns of the pulse wave signals PS1 and PS2 is smaller than α (NO in step S124), the process proceeds to step S125, and switches to set the weight of the transmission/reception antenna pair (TX2, RX2) to be large and the weight of the transmission/reception antenna pair (TX3, RX3) to be large in the respective first set of transmission/reception antenna pairs (41, 42) and second set of transmission/reception antenna pairs (44, 43). Thereafter, the process returns to step S101 in FIG. 16A to repeat the processing. Note that, in a case where a transmission/reception antenna pair suitable for use is not found even when the processing in FIGS. 16A to 16C is repeated a predetermined number of times, or a case where a transmitted/received antenna pair suitable for use is not found even after a predetermined period has elapsed, the CPU 100 displays an error on the display unit 50 and ends the process, in this example.

As described above, in the operation flow of FIGS. 16A to 16C, in the respective first set of transmission/reception antenna pairs (41, 42) and second set of transmission/reception antenna pairs (44, 43), firstly, the CPU 100 sequentially switches the weights of the transmission/reception antenna pairs (TX4, RX4) to (TX2, RX2) arranged at the right end in the longitudinal direction X of the belt 20 to be small as illustrated in FIGS. 17A to 17D, and then sequentially switches weights of the transmission/reception antenna pair (TX1, RX1) arranged at the left end to the transmission/reception antenna pairs (TX4, RX4) arranged at the right end to be larger as illustrated FIGS. 17D to 17H so as to search for a transmission/reception antenna pair with which the signal-to-noise ratio (S/N) becomes larger. Thereby, a transmission/reception antenna pair suitable for use can be reliably determined among the plurality of transmission/reception antenna pairs (TX1, RX1), (TX2, RX2), (TX3, RX3), and (TX4, RX4). Therefore, the signal-to-noise ratio (S/N) of the received signal can be increased, and as a result, the pulse wave signal, pulse wave transit time, and blood pressure as biological information can be accurately measured.

Further, in the first set of transmission/reception antenna pairs (41, 42) and the second set of transmission/reception antenna pairs (44, 43), during a process for weighting the respective transmission/reception antenna pairs (TX1, RX1), (TX2, RX2), (TX3, RX3), and (TX4, RX4), the switching can be stopped and the process can be completed when an acquired signal-to-noise ratio (S/N) is larger than the threshold value α. Therefore, the weighting process can be completed more quickly than α case where all the switching operations are performed.

In the above examples of FIGS. 16A to 16C, for the sake of simplicity, in the first set of transmission/reception antenna pairs (41, 42) and the second set of transmission/reception antenna pairs (44, 43), the weights of the transmission antennas TX1, TX2, TX3, and TX4 and the weights of the reception antennas RX1, RX2, RX3, and RX4 are respectively switched to be large (in this example, weight 1) or small (in this example, weight 0.1). However, this example does not set any limitation. The weights of the transmission antennas TX1, TX2, TX3, and TX4 and the weights of the reception antennas RX1, RX2, RX3, and RX4 can be arbitrarily set in the range from 0 to 1. In such a case, for example, in the four position displacement modes illustrated in FIG. 1I A to FIG. 1D, the results described in Table 5 below are obtained as optimum weights. In other words, as illustrated in FIG. 11A, in a case where the transmission/reception antenna group 40E is largely displaced to the left with respect to the radial artery 91, in this example, the weight of the transmission/reception antenna pair (TX1, RX1) is set as 0.174, the weight of the transmission/reception antenna pair (TX2, RX2) is set as 0.2, the weight of the transmission/reception antenna pair (TX3, RX3) is set as 0.4, and the weight of the transmission/reception antenna pair (TX4, RX4) is set as 1.0 in the first set of transmission/reception antenna pairs (41, 42) and second set of transmission/reception antenna pairs (44, 43). As illustrated in FIG. 11B, in a case where the transmission/reception antenna group 40E is slightly displaced to the left with respect to the radial artery 91, in this example, the weight of the transmission/reception antenna pair (TX1, RX1) is set as 0.1, the weight of the transmission/reception antenna pair (TX2, RX2) is set as 0.7, the weight of the transmission/reception antenna pair (TX3, RX3) is set as 1.0, and the weight of the transmission/reception antenna pair (TX4, RX4) is set as 0.6 in the first set of transmission/reception antenna pairs (41, 42) and second set of transmission/reception antenna pairs (44, 43). As illustrated in FIG. 11C, in a case where the transmission/reception antenna group 40E is slightly displaced to the right with respect to the radial artery 91, in this example, the weight of the transmission/reception antenna pair (TX1, RX1) is set as 1.0, the weight of the transmission/reception antenna pair (TX2, RX2) is set as 1.0, the weight of the transmission/reception antenna pair (TX3, RX3) is set as 0.3, and the weight of the transmission/reception antenna pair (TX4, RX4) is set as 0.1 in the first set of transmission/reception antenna pairs (41, 42) and second set of transmission/reception antenna pairs (44, 43). As illustrated in FIG. 11D, in a case where the transmission/reception antenna group 40E is largely displaced to the right with respect to the radial artery 91, in this example, the weight of the transmission/reception antenna pair (TX1, RX1) is set as 1.0, the weight of the transmission/reception antenna pair (TX2, RX2) is set as 0.1, the weight of the transmission/reception antenna pair (TX3, RX3) is set as 0.1, and the weight of the transmission/reception antenna pair (TX4, RX4) is set as 0.1 in the first set of transmission/reception antenna pairs (41, 42) and second set of transmission/reception antenna pairs (44, 43). Optimal weighting can be obtained by arbitrarily setting the weights of the transmission antennas TX1, TX2, TX3, and TX4 and the weights of the reception antennas RX1, RX2, RX3, and RX4 in the range from 0 to 1 in the above-described manner.

TABLE 5

| | TX1 | TX2 | TX3 | TX4 |
|---|---|---|---|---|
| Displacement manners | RX1 | RX2 | RX3 | RX4 |
| FIG. 11A | 0.1 | 0.2 | 0.4 | 1.0 |
| FIG. 11B | 0.1 | 0.7 | 1.0 | 0.6 |
| FIG. 11C | 1.0 | 1.0 | 0.3 | 0.1 |
| FIG. 11D | 1.0 | 0.1 | 0.1 | 0.1 |

In the example of FIGS. 16A to 16C above, for the sake of simplicity, the weights of the transmission/reception antenna pairs (TX1, RX1), (TX2, RX2), (TX3, RX3), and (TX4, RX4) included in the first set of transmission/reception antenna pairs (41, 42), and the weights of the transmission/reception antenna pairs (TX1, RX1), (TX2, RX2), (TX3, RX3), (TX4, RX4) included in the second set of transmission/reception antenna pairs (44, 43) can be switched to the same weight in conjunction with each other. However, this example does not set any limitation. The weighting of the transmission/reception antenna pair in the first set of transmission/reception antenna pairs (41, 42) and the weighting of the transmission/reception antenna pair in the second set of transmission/reception antenna pairs (44, 43) may be performed independently from each other. With this configuration, in a case where the belt 20 is worn to the left wrist 90, and the belt 20 obliquely intersects the radial artery 91 so that the transmission/reception antenna group 40E is obliquely displaced in the paper plane of FIG. 3 for example, a weight of a transmission/reception antenna pair suitable for use can be set respectively in the first set of transmission/reception antenna pairs (41, 42) and the second set of transmission/reception antenna pairs (44, 43). Therefore, the signal-to-noise ratio (S/N) of the received signal can be increased, and as a result, the pulse wave signal, pulse wave transit time, and blood pressure as biological information can be accurately measured.

### (Control of Function A)

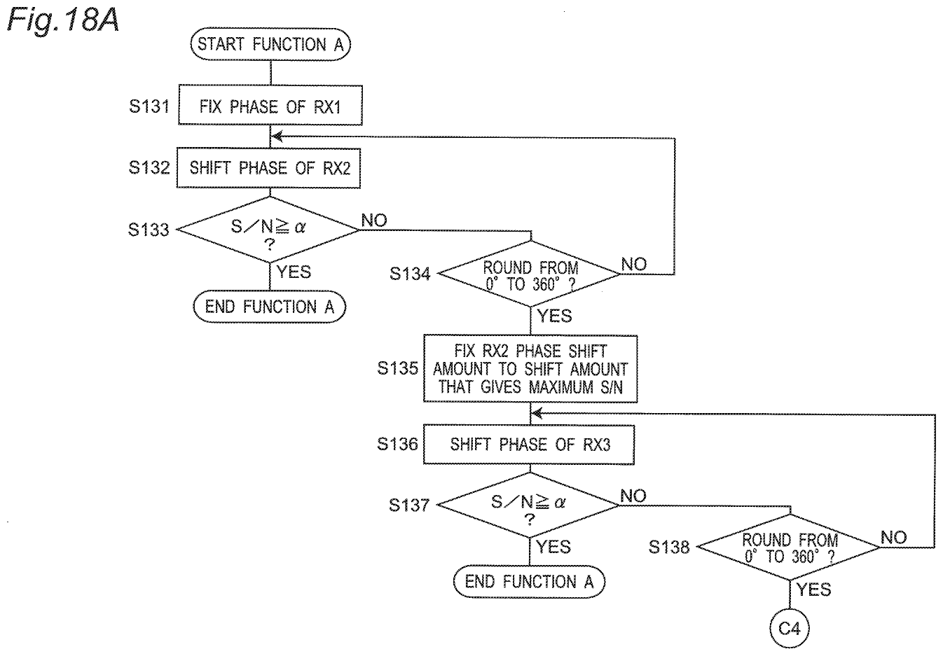

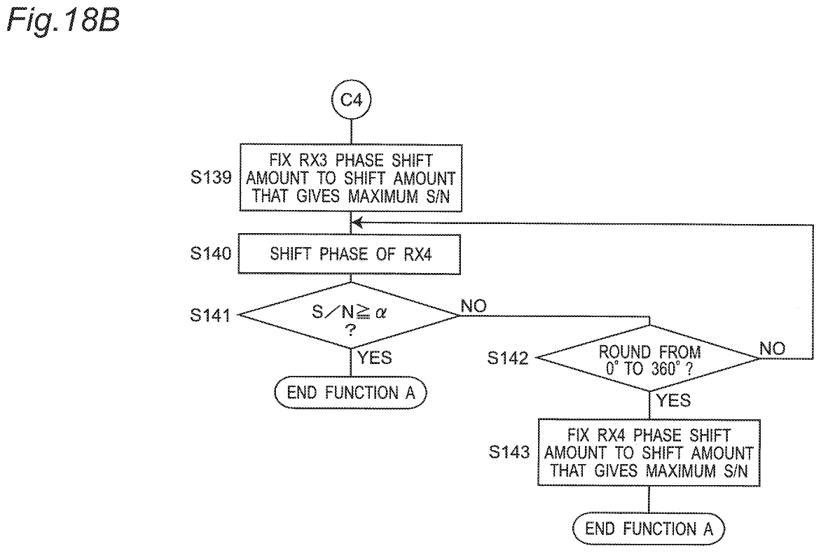

FIGS. 18A and 18B illustrate an operation flow of a case where the CPU 100 controls the function A illustrated in FIGS. 16A to 16C. FIGS. 18A and 18B illustrate the case where the relative phase of the signals received by the reception antennas RX1, RX2, RX3, and RX4 is shifted, however, processing of the same operation flow is performed when the relative phase of the radio wave emitted by the transmission antennas TX1, TX2, TX3, and TX4 are shifted. In the following description, it is assumed that when phase of an antenna element is not explicitly described as “shifted”, the phase is fixed.

More specifically, first, as described in step S131 of FIG. 18A, the phase of the reception antenna RX1 is fixed. Subsequently, as described in step S132, the phase of the reception antenna RX2 is started to be shifted relative to the phase of the reception antenna RX1. As described in step S133, in the process of shifting the phase of the reception antenna RX2, the CPU 100 acquires the signal-to-noise ratios (S/N) of the pulse wave signals PS1 and PS2, stores the S/Ns in the memory 51, and determines whether or not any of the acquired S/Ns are larger than the threshold value α. Here, in a case where both S/Ns are equal to or larger than α (YES in step S133), it is determined that the relative phase shift adjustment is completed, and the control of the function A is terminated.

On the other hand, in a case where any of S/Ns of the pulse wave signals PS1 and PS2 is smaller than α in step S133 (NO in step S133), the process proceeds to step S134 to determine whether or not the phase of the reception antenna RX2 has made a relative round from 0° to 360° with respect to the phase of the reception antenna RX1. In a case where the circuit has not made a round yet (NO in step S134), the process returns to step S132, and the processes in steps S132 to S134 are repeated. In a case where the phase of the reception antenna RX2 has made a round (YES in step S134), the process proceeds to step S135, and the phase shift amount of the reception antenna RX2 is fixed to a shift amount within the range from 0° to 360° with which the S/Ns of the pulse wave signals PS1 and PS2 becomes the maximum.

Next, as described in step S136, the phase of the reception antenna RX3 is started to be shifted relative to the phase of the reception antenna RX1. As described in step S137, in the process of shifting the phase of the reception antenna RX3, the CPU 100 acquires the signal-to-noise ratios (SI/N) of the pulse wave signals PS1 and PS2, stores the S/Ns in the memory 51, and determines whether or not any of the acquired S/Ns are larger than the threshold value α. Here, in a case where the both S/Ns are equal to or larger than α (YES in step S137), it is determined that the relative phase shift adjustment has been completed, and the control of the function A is terminated.

On the other hand, in a case where any of S/Ns of the pulse wave signals PS1 and PS2 is smaller than α in step S137 (NO in step S137), the process proceeds to step S138 to determine whether or not the phase of the reception antenna RX3 has made a relative round from 0° to 360° with respect to the phase of the reception antenna RX1. In a case where the circuit has not made a round yet (NO in step S138), the process returns to step S136, and the processes in steps S136 to S138 are repeated. In a case where the phase of the reception antenna RX3 has made a round (YES in step S138), the process proceeds to step S139 in FIG. 18B, and the phase shift amount of the reception antenna RX3 is fixed to a shift amount within the range from 00 to 360° with which the S/Ns of the pulse wave signals PS1 and PS2 becomes the maximum.

Next, as described in step S140, the phase of the reception antenna RX4 is started to be shifted relative to the phase of the reception antenna RX1. As described in step S141, in the process of shifting the phase of the reception antenna RX4, the CPU 100 acquires the signal-to-noise ratios (S/N) of the pulse wave signals PS1 and PS2, stores the S/Ns in the memory 51, and determines whether or not any of the acquired S/Ns are larger than the threshold value α. Here, in a case where the both S/Ns are equal to or larger than α (YES in step S141), it is determined that the relative phase shift adjustment has been completed, and the control of the function A is terminated.

On the other hand, in a case where any of S/Ns of the pulse wave signals PS1 and PS2 is smaller than α in step S141 (NO in step S141), the process proceeds to step S142 to determine whether or not the phase of the reception antenna RX4 has made a relative round from 0° to 360° with respect to the phase of the reception antenna RX1. In a case where the circuit has not made a round yet (NO in step S142), the process returns to step S140 and the processes in steps S140 to S142 are repeated. In a case where the phase of the reception antenna RX4 has made a round (YES in step S142), the process proceeds to step S143, and the phase shift amount of the reception antenna RX4 is fixed to a shift amount within the range from 0° to 360° with which the S/Ns of the pulse wave signals PS1 and PS2 becomes the maximum. Thereby, the control of the function A is finished.

As described above, this operation flow (control of function A) is also applied when shifting the relative phase of radio waves emitted by the transmission antennas TX1, TX2, TX3, and TX4.

In this manner, in the above operation flow (control of function A), the CPU 100 shifts the relative phase of the radio waves emitted by the transmission antennas TX1, TX2, TX3, and TX4 and the relative phase of the signals received by the reception antennas RX1, RX2, RX3, and RX4, respectively in the first set of transmission/reception antenna pairs (41, 42) and second set of transmission/reception antenna pairs (44, 43), and increases the signal-to-noise ratios (S/N) of the pulse wave signals PS1 and PS2 as a combined signal obtained by combining the signals. Therefore, the phase shift among the received signals is adjusted, and the signal-to-noise ratio (S/N) can be further improved.

### (Control of Function C)

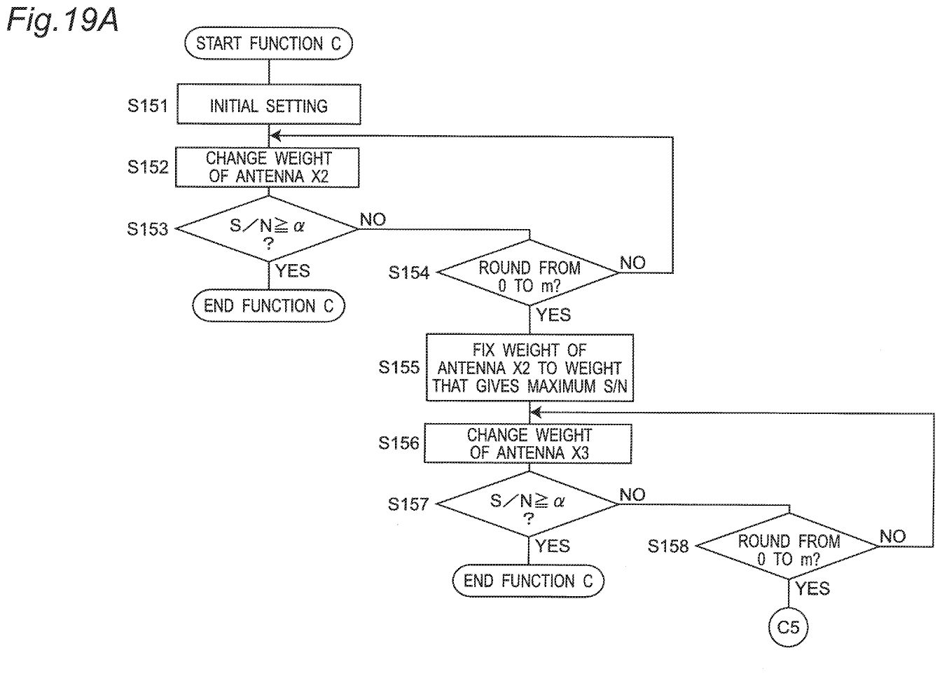

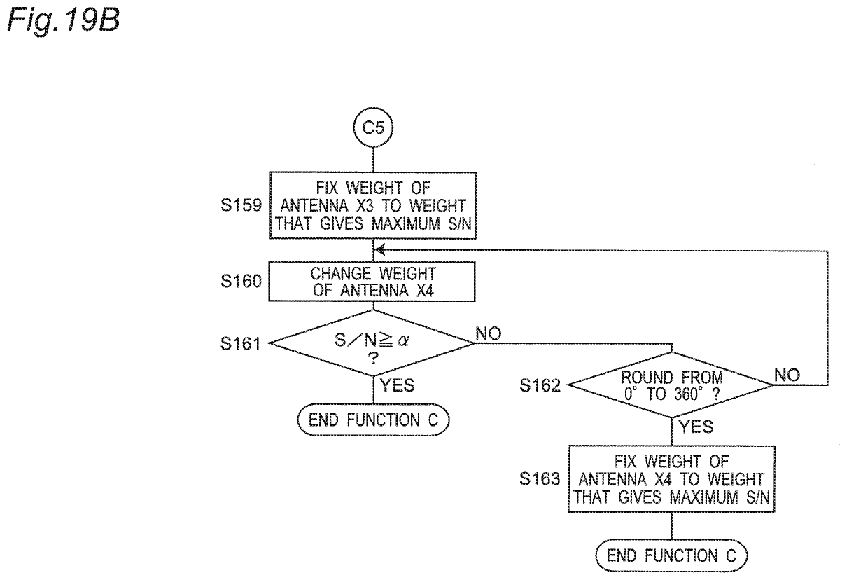

FIGS. 19A and 19B illustrate an operation flow of a case where the CPU 100 controls the function C illustrated in FIGS. 16A to 16C. In this operation flow, the antenna having the lowest weight in the main flow (FIG. 10) is X1, and the other antennas are X2, X3, and X4. Here, the antennas X1, X2, X3, and X4 are any of the transmission antennas TX1, TX2, TX3, and TX4 or the reception antennas RX1, RX2, RX3, and RX4. In the following description, it is assumed that when weight of an antenna element is not explicitly described as “changed”, the weight is fixed.

More specifically, first, as described in step S151 of FIG. 19A, initial setting is performed. In this initial setting, the weight of the antenna X1 is fixed, and the initial weights of the other antennas X2, X3, and X4 are set to the maximum weight m (=1).

Subsequently, as described in step S152, the weight of the antenna X2 is started to be changed. As described in step S153, in the process of changing the weight of the antenna X2, the CPU 100 acquires the signal-to-noise ratios (S/Ns) of the pulse wave signals PS1 and PS2, stores the S/Ns in the memory 51, and determines whether or not the acquired S/Ns are larger than the threshold value α. Here, in a case where the S/Ns are both equal to or larger than α (YES in step S153), it is determined that the adjustment of the relative weight among the received signals is completed, and the control of the function C is ended.

On the other hand, in a case where any of the S/Ns of the pulse wave signals PSI and PS2 is smaller than α in step S153 (NO in step S153), the process proceeds to step S154 to determine whether or not changing the weight of the antenna X2 has made a round from 0 to m. In a case where the round had not made yet (NO in step S154), the process returns to step S152 to repeat the processes in steps S152 to S154. When the weighting of the antenna X2 has made a round (YES in step S154), the process proceeds to step S155, and the weight of the antenna X2 is fixed to the weight within the range from 0 to m with which the S/Ns of pulse wave signals PS1 and PS2 becomes maximum.

Next, as described in step S156, the weight of the antenna X3 is started to be changed. As described in step S157, in the process of changing the weight of the antenna X3, the CPU 100 acquires the signal-to-noise ratios (S/Ns) of the pulse wave signals PS1 and PS2, stores the S/Ns in the memory 51, and determines whether or not the acquired S/Ns are larger than the threshold value α. Here, in a case where the S/Ns are both equal to or larger than α (YES in step S157), it is determined that the adjustment of the relative weight among the received signals is completed, and the control of the function C is ended.

On the other hand, in a case where any of the S/Ns of the pulse wave signals PS1 and PS2 is smaller than α in step S157 (NO in step S157), the process proceeds to step S158 to determine whether or not changing the weight of the antenna X3 has made a round from 0 to m. In a case where the round had not made yet (NO in step S158), the process returns to step S156 to repeat the processes in steps S156 to S158. When the weighting of antenna X3 has made a round (YES in step S158), the process proceeds to step S159 in FIG. 19B, and the weight of antenna X3 is fixed to the weight within the range from 0 to m with which the S/Ns of pulse wave signals PS1 and PS2 becomes maximum.

Next, as described in step S160, the weight of the antenna X4 is started to be changed. As described in step S161, in the process of changing the weight of the antenna X4, the CPU 100 acquires the signal-to-noise ratios (S/Ns) of the pulse wave signals PS1 and PS2, stores the S/Ns in the memory 51, and determines whether or not the acquired S/Ns are larger than the threshold value α. Here, in a case where the S/Ns are both equal to or larger than α (YES in step S161), it is determined that the adjustment of the relative weight among the received signals is completed, and the control of the function C is ended.

On the other hand, in a case where any of the S/Ns of the pulse wave signals PSI and PS2 is smaller than α in step S161 (NO in step S161), the process proceeds to step S162 to determine whether or not changing the weight of the antenna X4 has made a round from 0 to m. In a case where the round had not made yet (NO in step S162), the process returns to step S160 to repeat the processes in steps S160 to S162. When the weighting of antenna X4 has made a round (YES in step S162), the process proceeds to step S163, and the weight of antenna X4 is fixed to the weight within the range from 0 to m with which the S/Ns of pulse wave signals PS1 and PS2 becomes maximum. Thereby, the control of the function C is finished.

This operation flow (control of function C) is applied in a case where changing is made on the relative weight among the radio waves emitted by the transmission antennas TX1, TX2, TX3, and TX4 and the relative weight among the signals respectively received by the reception antennas RX1, RX2, RX3, and RX4.

In this manner, in the above operation flow (control of function A), the CPU 100 changes the relative weights of the radio waves emitted by the transmission antennas TX1, TX2, TX3, and TX4 and the relative weights of the signals received by the reception antennas RX1, RX2, RX3, and RX4, respectively in the first set of transmission/reception antenna pairs (41, 42) and second set of transmission/reception antenna pairs (44, 43), and increases the signal-to-noise ratio (S/N) of the pulse wave signals PS1 and PS2 as a combined signal obtained by combining the signals. Therefore, the relative weights among the received signals is adjusted, and the signal-to-noise ratio (S/N) can be further improved.

### (Example of Weighting for Two Rows and Two Columns of Transmission/Reception Antennas)

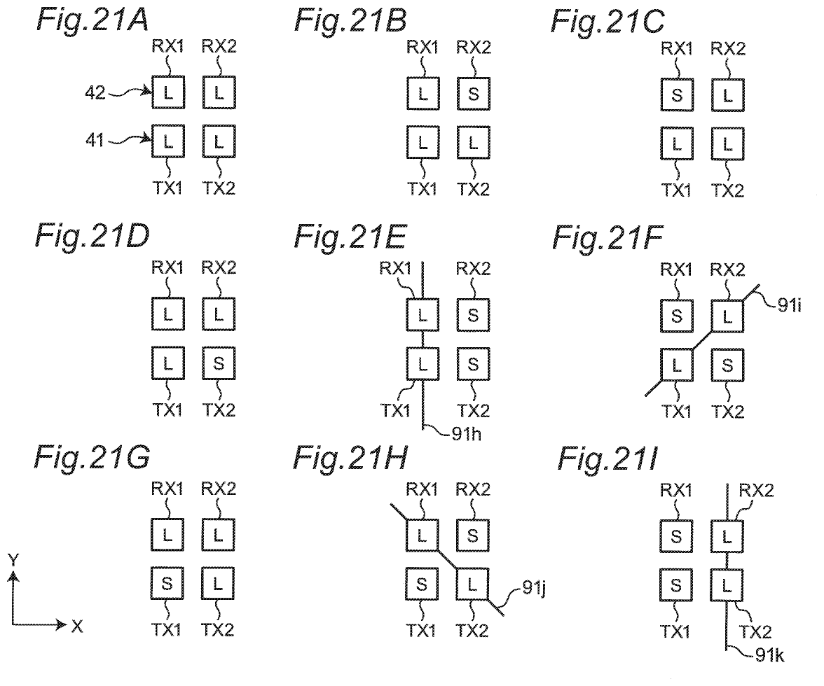

This example focuses on the two transmission antennas TX1 and TX2 arranged along the longitudinal direction X of the belt 20 and the two reception antennas RX1 and RX2 arranged spaced apart from each other along the longitudinal direction X of the belt 20 in the first set of transmission/reception antenna pairs (41, 42), as illustrated in FIG. 21A, as antenna elements arranged spaced apart from each other in two rows by two columns in the transmission/reception antenna group 40E of the transmission/reception unit 40.

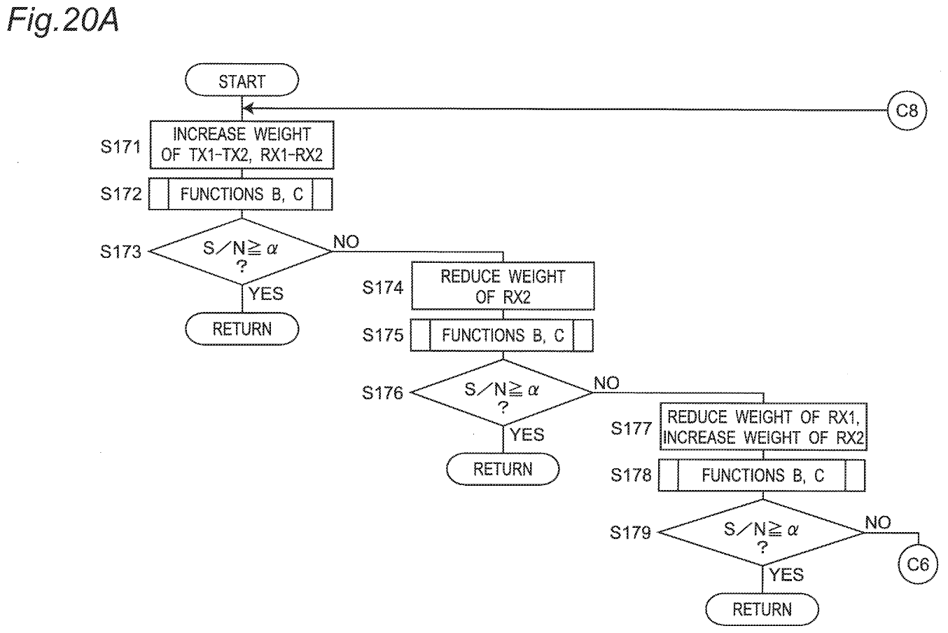

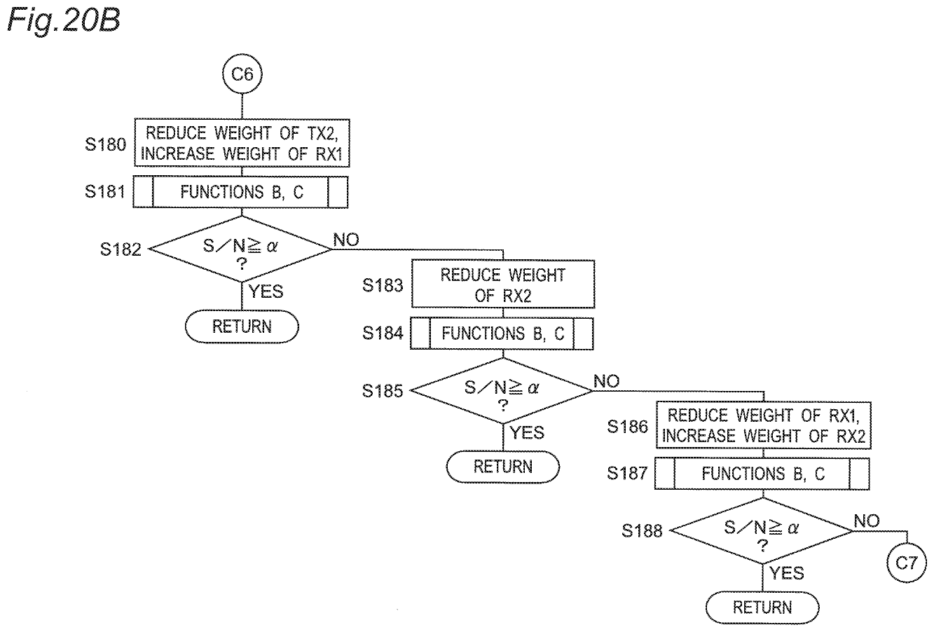

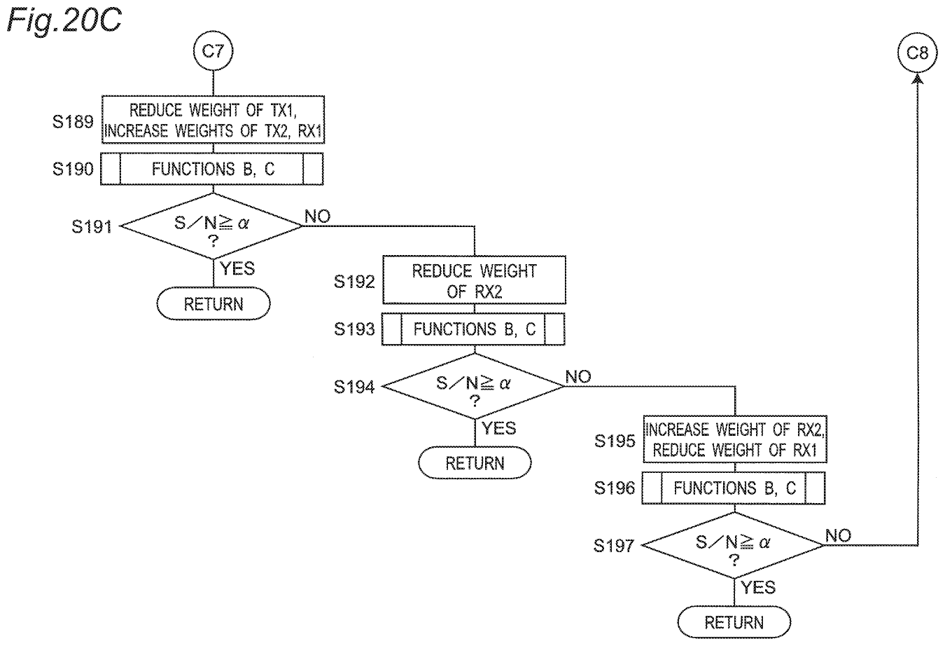

In this example, basically the same operation flow illustrated in FIG. 10 is executed for blood pressure measurement based on the pulse wave transit time. Then, in step S12 of FIG. 10, while performing the above-described transmission and reception, the CPU 100 serves as the antenna control unit 111, and controls to weight the antenna elements of the above two rows and two columns as illustrated in FIGS. 20A to 20C.

In the examples of FIGS. 20A to 20C, the weights of the transmission antennas TX1 and TX2 and reception antennas RX1 and RX2 are switched to large (weight 1 in this example) or small (weight 0.1 in this example).

More specifically, first, as described in step S171 of FIG. 20A, the CPU 100 serves as the antenna control unit 111, and sets the weights of all the transmission antennas TX1 and TX2 and the reception antenna RX1 and RX2 to large, in the first set of transmission/reception antenna pairs (41, 42). FIG. 21A schematically illustrates the weighting state. In accordance with this weighting, the CPU 100 serves as the pulse wave detection unit 101 to acquire a pulse wave signal PS1 indicating the pulse wave of the corresponding portion of the radial artery 91 described above.

Next, as described in step S172 of FIG. 20A, the CPU 100 serves as the antenna control unit 111 and controls to shift the relative phases of the radio waves emitted by the transmission antennas TX1 and TX2 and the relative phases of the signals received by the reception antennas RX1 and RX2 and increase the signal-to-noise ratio (S/N) of the combined signal obtained by combining the signals (this is referred to as “control of function B”). The control of the function B will be described in detail later. In addition, the control (control of function C) is performed to change the relative weight of the radio waves emitted by the transmission antennas TX1 and TX2 and the relative weight of the signals received by the reception antennas RX1 and RX2 and increase the signal-to-noise ratio (S/N) of a combined signal obtained by combining the signals. The control of the function C is the same as the control already described with reference to FIGS. 19A and 19B.

Next, as described in step S173 of FIG. 20A, the CPU 100 serves as the antenna control unit 111 to acquire the signal-to-noise ratio (S/N) of the pulse wave signal PS1 and determine whether or not the acquired S/N is larger than the threshold value α as a reference value (in this example, a is set to 40 dB in advance. The same applies below.). Here, in a case where the S/N is equal to or larger than α (YES in step S173), it is determined that the current weighting of the transmission/reception antenna pair is appropriate, and the process returns to the main flow (FIG. 10).

On the other hand, in a case where the S/N is smaller than α in step S173 of FIG. 20A (NO in step S173), the process proceeds to step S174, and the CPU 100 serves as the antenna control unit 111, and switches to set the weight of the reception antenna RX2 to a small level in the first set of transmission/reception antenna pairs (41, 42). Thereby, as schematically illustrated in FIG. 21B, in the first set of transmission/reception antenna pairs (41, 42), the weights of the transmission antennas TX1 and TX2 and the reception antenna RX1 are large, and the weight of the reception antenna RX2 is small. In accordance with this weighting, the CPU 100 serves as the pulse wave detection unit 101 to acquire a pulse wave signal PS1 indicating the pulse wave of the corresponding portion of the radial artery 91 described above.

Next, as described in step S175 in FIG. 20A, the CPU 100 serves as the antenna control unit 111 to control the above-described function B and function C.

Next, as described in step S176, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S176), it is determined that the current weighting of the transmission/reception antenna pair is appropriate, and the process returns to the main flow (FIG. 10).

On the other hand, in a case where the S/N is smaller than α in step S176 of FIG. 20A (NO in step S176), the process proceeds to step S177, and the CPU 100 serves as the antenna control unit 111 to switch to set the weight of the reception antenna RX1 to small and switch to set the weight of the reception antenna RX2 to large. Thereby, as schematically illustrated in FIG. 21C, in the first set of transmission/reception antenna pairs (41, 42), the weights of the transmission antennas TX1 and TX2 and the reception antenna RX2 are large, and the weight of the reception antenna RX1 is small. In accordance with this weighting, the CPU 100 serves as the pulse wave detection unit 101 to acquire a pulse wave signal PS1 indicating the pulse wave of the corresponding portion of the radial artery 91 described above.

Next, as described in step S178 in FIG. 20A, the CPU 100 serves as the antenna control unit 111 to control the above-described function B and function C.

Next, as described in step S179, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S179), it is determined that the current weighting of the transmission/reception antenna pair is appropriate, and the process returns to the main flow (FIG. 10).

On the other hand, in a case where the S/N is smaller than α in step S179 of FIG. 20A (NO in step S179), the process proceeds to step S180 in FIG. 20B, and the CPU 100 serves as the antenna control unit 111 to switch to set the weight of the transmission antenna TX2 to small and switch to set the weight of the reception antenna RX1 to large. Thereby, as schematically illustrated in FIG. 21D, in the first set of transmission/reception antenna pairs (41, 42), the weights of the transmission antennas TX1 and reception antenna RX1 and RX2 are large, and the weight of the transmission antenna TX2 is small. In accordance with this weighting, the CPU 100 serves as the pulse wave detection unit 101 to acquire a pulse wave signal PS1 indicating the pulse wave of the corresponding portion of the radial artery 91 described above.

Next, as described in step S181 in FIG. 20B, the CPU 100 serves as the antenna control unit 111 to control the above-described function B and function C.

Next, as described in step S182, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S182), it is determined that the current weighting of the transmission/reception antenna pair is appropriate, and the process returns to the main flow (FIG. 10).

On the other hand, in a case where the S/N is smaller than α in step S182 of FIG. 20B (NO in step S182), the process proceeds to step S183, and the CPU 100 serves as the antenna control unit 111, and switches to set the weight of the reception antenna RX2 to small. Thereby, as schematically illustrated in FIG. 21E, in the first set of transmission/reception antenna pairs (41, 42), the weights of the transmission antenna TX1 and the reception antenna RX1 are large, and the transmission antenna TX2 and the reception antenna RX2 are small (first setting). In accordance with this weighting, the CPU 100 serves as the pulse wave detection unit 101 to acquire a pulse wave signal PSI indicating the pulse wave of the corresponding portion of the radial artery 91 described above.

Next, as described in step S184 in FIG. 20B, the CPU 100 serves as the antenna control unit 111 to control the above-described function B and function C.

Next, as described in step S185, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S185), it is determined that the current weighting of the transmission/reception antenna pair is appropriate, and the process returns to the main flow (FIG. 10). For example, as illustrated by a straight line 91h in FIG. 21E, this case may correspond to a case where the radial artery 91 corresponds to the transmission antenna TX1 and the reception antenna RX1.

On the other hand, in a case where the S/N is smaller than α in step S185 of FIG. 20B (NO in step S185), the process proceeds to step S186, and the CPU 100 serves as the antenna control unit 111 to switch to set the weight of the reception antenna RX1 to small and switch to set the weight of the reception antenna RX2 to large. Thereby, as schematically illustrated in FIG. 21F, in the first set of transmission/reception antenna pairs (41, 42), the weights of the transmission antenna TX1 and reception antenna RX2 are large, and the transmission antenna TX2 and reception antenna RX1 are small (third setting). In accordance with this weighting, the CPU 100 serves as the pulse wave detection unit 101 to acquire a pulse wave signal PS1 indicating the pulse wave of the corresponding portion of the radial artery 91 described above.

Next, as described in step S187 in FIG. 20B, the CPU 100 serves as the antenna control unit 111 to control the above-described function B and function C.

Next, as described in step S188, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S188), it is determined that the current weighting of the transmission/reception antenna pair is appropriate, and the process returns to the main flow (FIG. 10). For example, as illustrated by a straight line 91i in FIG. 21F, this case may correspond to a case where the radial artery 91 diagonally corresponds to the transmission antenna TX1 and the reception antenna RX2.

On the other hand, in a case where the S/N is smaller than α in step S188 of FIG. 20B (NO in step S188), the process proceeds to step S189 in FIG. 20C, and the CPU 100 serves as the antenna control unit 111 to switch to set the weight of the transmission antenna TX1 to small and switch to set the weights of the transmission antenna TX2 and reception antenna RX1 to large. Thereby, as schematically illustrated in FIG. 21G, in the first set of transmission/reception antenna pairs (41, 42), the weights of the transmission antennas TX2 and reception antenna RX1 and RX2 are large, and the weight of the transmission antenna TX1 is small. In accordance with this weighting, the CPU 100 serves as the pulse wave detection unit 101 to acquire a pulse wave signal PS1 indicating the pulse wave of the corresponding portion of the radial artery 91 described above.

Next, as described in step S190 in FIG. 20C, the CPU 100 serves as the antenna control unit 111 to control the above-described function B and function C.

Next, as described in step S191, the CPU 100 serves as the antenna control unit Ill to acquire the signal-to-noise ratio (S/N) of the pulse wave signal PS1, and determine whether or not the acquired S/N is larger than the threshold value α. Here, in a case where the S/Ns is equal to or larger than α (YES in step S191), it is determined that the current weighting of the transmission/reception antenna pair is appropriate, and the process returns to the main flow (FIG. 10).

On the other hand, in a case where the S/N is smaller than α in step S191 of FIG. 20C (NO in step S191), the process proceeds to step S192, and the CPU 100 serves as the antenna control unit 111, and switches to set the weight of the reception antenna RX2 to small. Thereby, as schematically illustrated in FIG. 21H, in the first set of transmission/reception antenna pairs (41, 42), the weights of the transmission antenna TX2 and reception antenna RX1 are large, and the transmission antenna TX1 and reception antenna RX2 are small (fourth setting). In accordance with this weighting, the CPU 100 serves as the pulse wave detection unit 101 to acquire a pulse wave signal PSI indicating the pulse wave of the corresponding portion of the radial artery 91 described above.

Next, as described in step S193 in FIG. 20C, the CPU 100 serves as the antenna control unit 111 to control the above-described function B and function C.

Next, as described in step S194, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S194), it is determined that the current weighting of the transmission/reception antenna pair is appropriate, and the process returns to the main flow (FIG. 10). For example, as illustrated by a straight line 91j in FIG. 21H, this case may correspond to a case where the radial artery 91 diagonally corresponds to the reception antenna RX2 and the transmission antenna TX1.

On the other hand, in a case where the S/N is smaller than α in step S194 of FIG. 20C (NO in step S194), the process proceeds to step S195, and the CPU 100 serves as the antenna control unit 111 to switch to set the weight of the reception antenna RX2 to large and switch to set the weight of the reception antenna RX1 to small. Thereby, as schematically illustrated in FIG. 21I, in the first set of transmission/reception antenna pairs (41, 42), the weights of the transmission antenna TX2 and reception antenna RX2 are large, and the transmission antenna TX1 and reception antenna RX1 are small (second setting). In accordance with this weighting, the CPU 100 serves as the pulse wave detection unit 101 to acquire a pulse wave signal PS1 indicating the pulse wave of the corresponding portion of the radial artery 91 described above.

Next, as described in step S196 in FIG. 20C, the CPU 100 serves as the antenna control unit 111 to control the above-described function B and function C.

Next, as described in step S197, the CPU 100 serves as the antenna control unit 111 to acquire the signal-to-noise ratio (S/N) of the pulse wave signal PS1, and determine whether or not the acquired S/N is larger than the threshold value α. Here, in a case where the S/N is equal to or larger than α (YES in step S197), it is determined that the current weighting of the transmission/reception antenna pair is appropriate, and the process returns to the main flow (FIG. 10). For example, as illustrated by a straight line 91k in FIG. 21I, this case may correspond to a case where the radial artery 91 corresponds to the transmission antenna TX2 and the reception antenna RX2.

On the other hand, in a case where the S/N is smaller than α in step S197 in FIG. 20C (NO in step S197), the process returns to step S171 in FIG. 20A and the process is repeated. Note that, in a case where weighting of a transmission/reception antenna pair suitable for use is not found even when the processing in FIGS. 20A to 20C is repeated a predetermined number of times, or a case where weighting of a transmitted/received antenna pair suitable for use is not found even after a predetermined period has elapsed, the CPU 100 displays an error on the display unit 50 and ends the process, in this example.

As described above, in the operation flow of FIGS. 20A to 20C, regarding the two transmission antennas TX1 and TX2 and the two reception antennas RX1 and RX2 arranged spaced apart from each other along the longitudinal direction X of the belt 20, the CPU 100 executes processing by switching between a first setting (setting in FIG. 21E) that relatively increases the weight of the first transmission antenna TX1 and the first reception antenna RX1 arranged on the left side in the longitudinal direction X of the belt 20 and a second setting (setting in FIG. 21I) that relatively increases the weight of the second transmission antenna TX2 and the second reception antenna RX2 arranged on the right side in the longitudinal direction X of the belt 20. With this configuration, in a case where the belt 20 is worn to the left wrist 90, even when a position displacement of the transmission/reception antenna group 40E occurs in the circumferential direction with respect to the left wrist 90, any one of the transmission/reception antenna pairs (TX1, RX1) and (TX2, RX2) can increase the signal-to-noise ratio (S/N) of the received signal, and as a result, the pulse wave signal as biological information can be accurately measured. Further, regarding the two transmission antennas TX1 and TX2 and the two reception antennas RX1 and RX2 arranged spaced apart from each other along the longitudinal direction X of the belt 20, the CPU 100 executes processing by switching between a third setting (setting in FIG. 21F) that relatively increases the weight of the first transmission antenna TX1 and the second reception antenna RX2 and a fourth setting (setting in FIG. 21H) that relatively increases the weight of the second transmission antenna TX2 and the first reception antenna RX1. With this configuration, in a case where the belt 20 is worn to the left wrist 90, even when the belt 20 obliquely intersects the radial artery 91 and the transmission/reception antenna group 40E is obliquely displaced in the sheet plane of FIG. 3 for example, the signal-to-noise ratio (S/N) of the received signal can be increased by one of the transmission/reception antenna pairs (TX1, RX2) and (TX2, RX1) and, as a result, a pulse wave signal as biological information can be accurately measured.

Note that the matrix of the antenna elements that are the target of the operation flow of FIGS. 20A to 20C is not limited to two rows and two columns, and may be a multi-rows and multi-columns. In this case, the CPU 100 performs the switching described above for one or more sets of two rows and two columns of antenna elements included in the multiple rows and multiple columns. Also, the two rows and two columns of antenna elements to be controlled do not need to be adjacent to each other, and another antenna element may be arranged between these antenna elements.

### (Control of Function B)

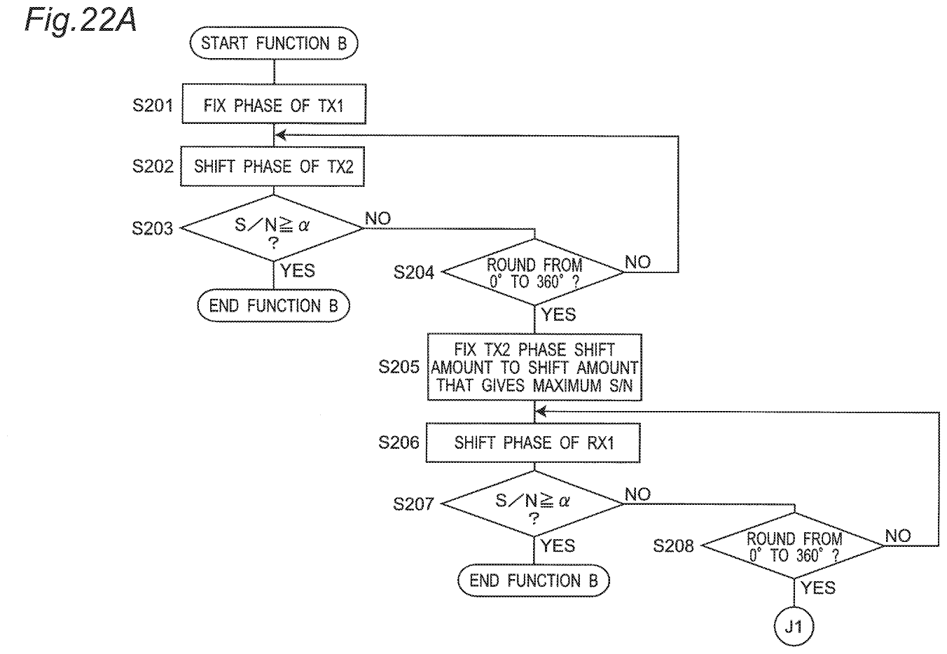

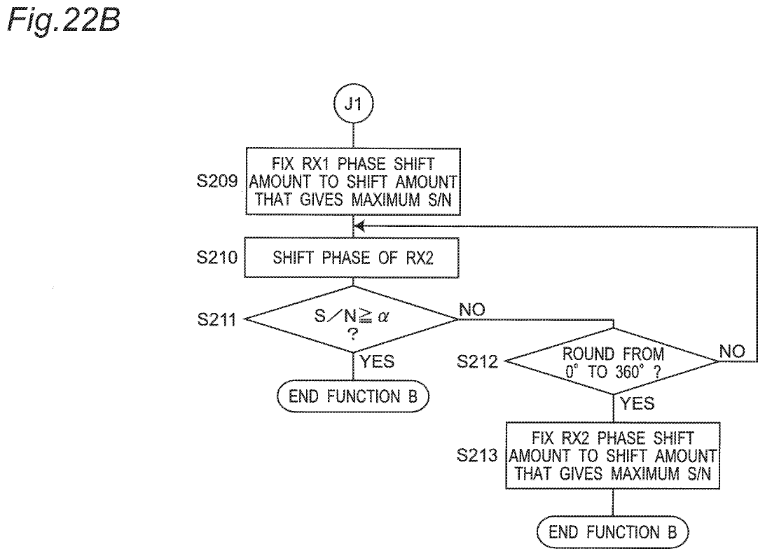

FIGS. 22A and 22B illustrate an operation flow of a case where the CPU 100 controls the function B illustrated in FIGS. 20A to 20C.

More specifically, first, as described in step S201 of FIG. 22A, the phase of the transmission antenna TX1 is fixed. Subsequently, as described in step S202, the phase of the transmission antenna TX2 is started to be shifted relative to the phase of the transmission antenna TX1. As described in step S203, in the process of shifting the phase of the transmission antenna TX2, the CPU 100 acquires the signal-to-noise ratio (S/N) of the pulse wave signal PS1, stores the S/N in the memory 51, and determines whether or not the acquired S/N is larger than the threshold value α. Here, in a case where the S/N is equal to or larger than α (YES in step S203), it is determined that the relative phase shift adjustment has been completed, and the control of the function B is terminated.

On the other hand, in a case where the S/N of the pulse wave signal PS1 is smaller than α in step S203 (NO in step S203), the process proceeds to step S204 to determine whether or not the phase of the transmission antenna TX2 has made a relative round from 0° to 360° with respect to the phase of the transmission antenna TX1. In a case where the circuit has not made a round yet (NO in step S204), the process returns to step S202, and the processes in steps S202 to S204 are repeated. In a case where the phase of the transmission antenna TX2 has made a round (YES in step S204), the process proceeds to step S205, and the phase shift amount of the transmission antenna TX2 is fixed to a shift amount within the range from 0° to 360° with which the S/N of the pulse wave signal PS1 becomes maximum.

Next, as described in step S206, the phase of the reception antenna RX1 is started to be shifted relative to the phase of the transmission antenna TX. As described in step S207, in the process of shifting the phase of the reception antenna RX1, the CPU 100 acquires the signal-to-noise ratio (S/N) of the pulse wave signal PS1, stores the S/N in the memory 51, and determines whether or not the acquired S/N is larger than the threshold value α. Here, in a case where the S/N is equal to or larger than α (YES in step S207), it is determined that the relative phase shift adjustment has been completed, and the control of the function B is terminated.

On the other hand, in a case where the S/N of the pulse wave signal PS1 is smaller than α in step S207 (NO in step S207), the process proceeds to step S208 to determine whether or not the phase of the reception antenna RX1 has made a relative round from 00 to 360° with respect to the phase of the transmission antenna TX1. In a case where the circuit has not made a round yet (NO in step S208), the process returns to step S206, and the processes in steps S206 to S208 are repeated. In a case where the phase of the reception antenna RX1 has made a round (YES in step S208), the process proceeds to step S209 in FIG. 22B, and the phase shift amount of the reception antenna RX1 is fixed to a shift amount within the range from 0° to 360° with which the S/N of the pulse wave signal PS1 becomes maximum.

Next, as described in step S210, the phase of the reception antenna RX2 is started to be shifted relative to the phase of the transmission antenna TX1. As described in step S211, in the process of shifting the phase of the reception antenna RX2, the CPU 100 acquires the signal-to-noise ratio (S/N) of the pulse wave signal PS1, stores the S/N in the memory 51, and determines whether or not the acquired S/N is larger than the threshold value α. Here, in a case where the S/N is equal to or larger than α (YES in step S211), it is determined that the relative phase shift adjustment has been completed, and the control of the function B is terminated.

On the other hand, in a case where the S/N of the pulse wave signal PS1 is smaller than α in step S211 (NO in step S211), the process proceeds to step S212 to determine whether or not the phase of the reception antenna RX2 has made a relative round from 00 to 360° with respect to the phase of the transmission antenna TX1. In a case where the circuit has not made a round yet (NO in step S212), the process returns to step S210, and the processes in steps S210 to S212 are repeated. In a case where the phase of the reception antenna RX4 has made a round (YES in step S212), the process proceeds to step S213, and the phase shift amount of the reception antenna RX4 is fixed to a shift amount within the range from 00 to 360° with which the S/N of the pulse wave signal PS1 becomes the maximum. Thereby, the control of the function B is finished.

In this manner, in this operation flow (control of function B), the CPU 100 shifts the relative phases of the radio waves emitted by the transmission antennas TX1 and TX2 and the relative phases of the signals received by the reception antennas RX1 and RX2 and increase the signal-to-noise ratio (S/N) of the pulse wave signal PS1 as the combined signal obtained by combining the signals. Therefore, the phase shift among the received signals is adjusted, and the signal-to-noise ratio (S/N) can be further improved.

### (Method for Dynamically Searching for Transmission/Reception Antenna Pair)

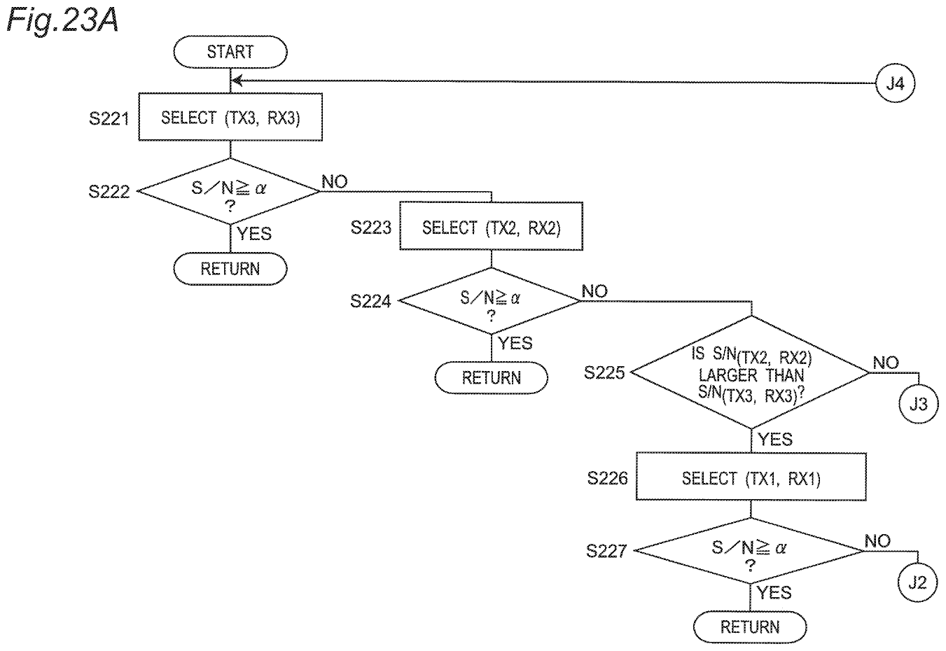

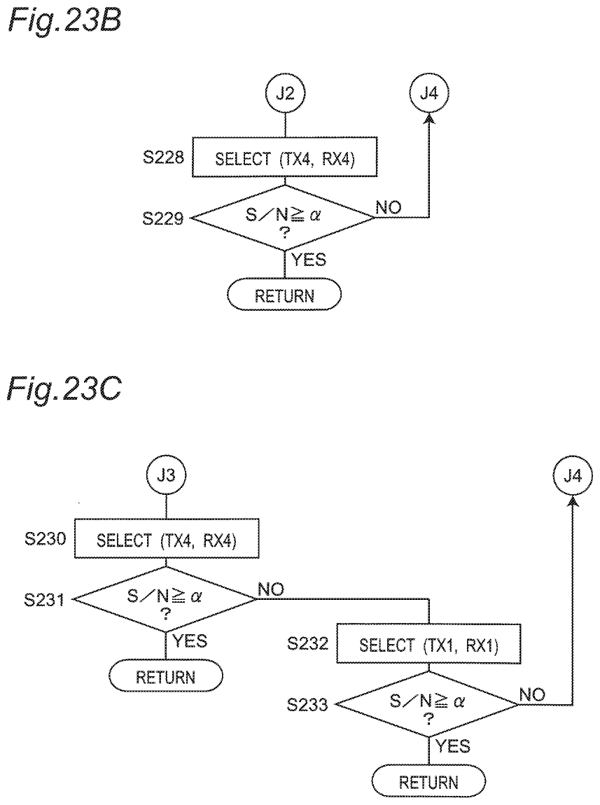

In the operation flows of FIGS. 12, 16A to 16C, and 20A to 20C described above, the order in which antenna elements are selected or weighted by switching is determined in advance. However, these examples do not set any limitation, and the order of selecting or weighting by switching antenna elements may be determined according to the condition of the signal-to-noise ratio (S/N). FIGS. 23A to 23C illustrate an operation flow of a case where antenna elements are selected by switching according to a signal-to-noise ratio (S/N) condition focusing on the transmission/reception antenna group 40E illustrated in FIG. 3.

First, the CPU 100 serves as the antenna control units 111 and 112, and in this example, as described in step S221 of FIG. 23A, the transmission/reception antenna pair (TX3, RX3) arranged at a substantially central portion in the longitudinal direction X of the belt 20 is selected from the transmission/reception antenna pairs (TX1, RX1), (TX2, RX2), (TX3, RX3), and (TX4, RX4) included in the first set of transmission/reception antenna pairs (41, 42), and the transmission/reception antenna pair (TX3, RX3) arranged at a substantially center portion in the longitudinal direction X of the belt 20 is selected form the transmission/reception antenna pairs (TX1, RX1), (TX2, RX2), (TX3, RX3), and (TX4, RX4) included in the second set of transmission/reception antenna pairs (44, 43) (corresponding to “first time” in Table 6 described later). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S222 of FIG. 23A, the CPU 100 serves as the antenna control units 111 and 112 to acquire the signal-to-noise ratio (S/N) of the pulse wave signals PS1 and PS2, stores the S/Ns in the memory 51, and determines whether or not the acquired S/Ns are all larger than the threshold values a as a reference value (In this example, α=40 dB is defined in advance. The same applies below.). Here, in a case where the both S/Ns are equal to or larger than α (YES in step S222), it is determined that the selection of the current transmission/reception antenna pairs is appropriate, and the process returns to the main flow (FIG. 10).

On the other hand, in a case where S/Ns in either the pulse wave signals PS1 and PS2 are smaller than α in step S222 of FIG. 23A (NO in step S222), the process proceeds to step S223, and the CPU 100 serves as the antenna control units 111 and 112 to select the transmission/reception antenna pair (TX2, RX2) located on the left side of (TX3, RX3) from the transmission/reception antenna pairs (TX1, RX1), (TX2, RX2), (TX3, RX3), and (TX4, RX4) included in the first set of transmission/reception antenna pairs (41, 42), and to select the transmission/reception antenna pair (TX2, RX2) located on the left side of (TX3, RX3) from the transmission/reception antenna pair (TX1, RX1), (TX2, RX2), (TX3, RX3), and (TX4, RX4) included in the second set of transmission/reception antenna pairs (44, 43) (equivalent to “second time” in Table 6 below). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S224 of FIG. 23A, the CPU 100 serves as the antenna control units 111 and 112, acquires the signal-to-noise ratios (S/N) of the pulse wave signals PS1 and PS2, stores the S/Ns in the memory 51, and determines whether or not the acquired S/Ns are larger than the threshold value α. Here, in a case where the both S/Ns are equal to or larger than α (YES in step S224), it is determined that the selection of the current transmission/reception antenna pairs is appropriate, and the process returns to the main flow (FIG. 10).

On the other hand, in a case where any of the S/Ns of the pulse wave signals PSI and PS2 are smaller than α in step S224 of FIG. 23A (NO in step S224), the process proceeds to step S225. Then, the CPU 100 serves as the antenna control units 111 and 112, and determines whether or not the signal-to-noise ratios of the pulse wave signals PSI and PS2 corresponding to the current selection (that is, the signal-to-noise ratio corresponding to the selection of the transmission/reception antenna pair (TX2, RX2) in step S223 in this example, which is expressed as S/N_{(TX2, RX2)}) is larger than the signal-to-noise ratios of the pulse wave signals PS1 and PS2 corresponding to the past selection (that is, the signal-to-noise ratio corresponding to the selection of the transmission/reception antenna pair (TX3, RX3) in step S221 in this example, which is expressed as S/N_{(TX3,RX3)}) stored in the memory 51.

Here, in a case where S/N_{(TX2,RX2)}) is larger than S/N_{(TX3,RX3)} in both of the pulse wave signals PS1 and PS2 (YES in step S225), the CPU 100 determines that the transmission/reception antenna pair (TX2, RX2) is likely to be displaced to the right from the radial artery 91. Here, the process proceeds to step S226, and the CPU 100 serves as the antenna control units 111 and 112 to select the transmission/reception antenna pair (TX1, RX1) located on the left side of (TX2, RX2) from the transmission/reception antenna pairs (TX1, RX1), (TX2, RX2), (TX3, RX3), and (TX4, RX4) included in the first set of transmission/reception antenna pairs (41, 42), and to select the transmission/reception antenna pair (TX1, RX1) located on the left side of (TX2, RX2) from the transmission/reception antenna pair (TX1, RX1), (TX2, RX2), (TX3, RX3), and (TX4, RX4) included in the second set of transmission/reception antenna pairs (44, 43) (equivalent to “third time” in Table 6 below). In response to this selection, the CPU 100 serves as the pulse wave detection units 101 and 102 to acquire pulse wave signals PS1 and PS2 indicating the pulse waves of the upstream portion 91u and the downstream portion 91d of the radial artery 91 described above.

Next, as described in step S227, the CPU 100 serves as the antenna control units 111 and 112, acquires the signal-to-noise ratios (S/N) of the pulse wave signals PS1 and PS2, stores the S/Ns in the memory 51, and determines whether or not the acquired S/Ns are larger than the threshold value α. Here, in a case where the both S/Ns are equal to or larger than α (YES in step S227), it is determined that the selection of the current transmission/reception antenna pair is appropriate, and the process returns to the main flow (FIG. 10).

On the other hand, in a case where S/Ns in either the pulse wave signals PS1 and PS2 are smaller than α in step S227 of FIG. 23A (NO in step S227), the process proceeds to step S228 in FIG. 23B, and the CPU 100 serves as the antenna control units 111 and 112 to select the remaining transmission/reception antenna pair (TX4, RX4) from the transmission/reception antenna pairs (TX1, RX1), (TX2, RX2), (TX3, RX3), and (TX4, RX4) included in the first set of transmission/reception antenna pairs (41, 42), and to select the remaining transmission/reception antenna pair (TX4, RX4) from the transmission/reception antenna pair (TX1, RX1), (TX2, RX2), (TX3, RX3), and (TX4, RX4) included in the second set of transmission/reception antenna pairs (44, 43) (equivalent to “fourth time” in Table 6 below). In response to this selection, the CPU 100 serves as the pulse wave detection units 101 and 102 to acquire pulse wave signals PSI and PS2 indicating the pulse waves of the upstream portion 91u and the downstream portion 91d of the radial artery 91 described above.

Next, as described in step S229 of FIG. 23B, the CPU 100 serves as the antenna control units 111 and 112, acquires the signal-to-noise ratios (S/N) of the pulse wave signals PS1 and PS2, stores the S/Ns in the memory 51, and determines whether or not the acquired S/Ns are larger than the threshold value α. Here, in a case where the both S/Ns are equal to or larger than α (YES in step S229), it is determined that the selection of the current transmission/reception antenna pair is appropriate, and the process returns to the main flow (FIG. 10).

On the other hand, in a case where S/Ns in either of the pulse wave signals PS1 and PS2 are smaller than α in step S229 of FIG. 23B (NO in step S229), the process returns to step S221 of FIG. 23A and the process is repeated.

Contrary to the above flow, in a case where S/N_{(TX3,RX3)} is larger than S/N_{(TX2,RX2)}) in both of the pulse wave signals PS1 and PS2 in step S225 in FIG. 23A (NO in step S225), the CPU 100 determines that the transmission/reception antenna pair (TX3, RX3) is likely to be displaced to the left from the radial artery 91. Here, the process proceeds to step S230 pf FIG. 23C, and the CPU 100 serves as the antenna control units 111 and 112 to select the transmission/reception antenna pair (TX4, RX4) located on the right side of (TX3, RX3) from the transmission/reception antenna pairs (TX1, RX1), (TX2, RX2), (TX3, RX3), and (TX4, RX4) included in the first set of transmission/reception antenna pairs (41, 42), and to select the transmission/reception antenna pair (TX4, RX4) located on the right side of (TX3, RX3) from the transmission/reception antenna pair (TX1, RX1), (TX2, RX2), (TX3, RX3), and (TX4, RX4) included in the second set of transmission/reception antenna pairs (44, 43) (equivalent to “third time” in Table 7 below). In response to this selection, the CPU 100 serves as the pulse wave detection units 101 and 102 to acquire pulse wave signals PSI and PS2 indicating the pulse waves of the upstream portion 91u and the downstream portion 91d of the radial artery 91 described above. Note that the selections of“first time” and “second time” in Table 7 are the same as in Table 6.

Next, as described in step S231 of FIG. 23C, the CPU 100 serves as the antenna control units 111 and 112, acquires the signal-to-noise ratios (S/N) of the pulse wave signals PS1 and PS2, stores the S/Ns in the memory 51, and determines whether or not the acquired S/Ns are larger than the threshold value α. Here, in a case where the both S/Ns are equal to or larger than α (YES in step S231), it is determined that the selection of the current transmission/reception antenna pair is appropriate, and the process returns to the main flow (FIG. 10).

On the other hand, in a case where S/Ns in either the pulse wave signals PS1 and PS2 are smaller than α in step S231 of FIG. 23C (NO in step S231), the process proceeds to step S232, and the CPU 100 serves as the antenna control units 111 and 112 to select the remaining transmission/reception antenna pair (TX1, RX1) from the transmission/reception antenna pairs (TX1, RX1), (TX2, RX2), (TX3, RX3), and (TX4, RX4) included in the first set of transmission/reception antenna pairs (41, 42), and to select the remaining transmission/reception antenna pair (TX1, RX1) from the transmission/reception antenna pair (TX1, RX1), (TX2, RX2), (TX3, RX3), and (TX4, RX4) included in the second set of transmission/reception antenna pairs (44, 43) (equivalent to “fourth time” in Table 7 below). In response to this selection, the CPU 100 serves as the pulse wave detection units 101 and 102 to acquire pulse wave signals PSI and PS2 indicating the pulse waves of the upstream portion 91u and the downstream portion 91d of the radial artery 91 described above.

Next, as described in step S233 of FIG. 23C, the CPU 100 serves as the antenna control units 111 and 112, acquires the signal-to-noise ratios (S/N) of the pulse wave signals PS1 and PS2, stores the S/Ns in the memory 51, and determines whether or not the acquired S/Ns are larger than the threshold value α. Here, in a case where the both S/Ns are equal to or larger than α (YES in step S233), it is determined that the selection of the current transmission/reception antenna pair is appropriate, and the process returns to the main flow (FIG. 10).

On the other hand, in a case where S/Ns in either of the pulse wave signals PS1 and PS2 are smaller than α in step S233 of FIG. 23C (NO in step S233), the process returns to step S221 of FIG. 23A and the process is repeated. Note that, in a case where a transmission/reception antenna pair suitable for use is not found even when the processing in FIGS. 23A to 23C is repeated a predetermined number of times, or a case where a transmitted/received antenna pair suitable for use is not found even after a predetermined period has elapsed, the CPU 100 displays an error on the display unit 50 and ends the process, in this example.

Note that, in this operation flow, for the sake of simplicity, in step S225 in FIG. 23A, it is assumed that S/N_{(TX2,RX2)} is larger than S/N_{(TX3,RX3)} in both of the pulse wave signals PS1 and PS2, or S/N_{(TX2,RX2)} is smaller than S/N_{(TX3,RX3)} in both of the wave signals PS1 and PS2.

TABLE 6

| | TX1 | TX2 | TX3 | TX4 |
|---|---|---|---|---|
| Number of Times | RX1 | RX2 | RX3 | RX4 |
| First time | — | — | Select | — |
| Second time | — | Select | — | — |
| Third time | Select | — | — | — |
| Fourth time | — | — | — | Select |

TABLE 7

| | TX1 | TX2 | TX3 | TX4 |
|---|---|---|---|---|
| Number of Times | RX1 | RX2 | RX3 | RX4 |
| First time | — | — | Select | — |
| Second time | — | Select | — | — |
| Third time | — | — | — | Select |
| Fourth time | Select | — | — | — |

Thus, in the operation flow of FIGS. 23A to 23C, each time the CPU 100 switches the selection once, the signal-to-noise ratio (S/N) of the signal received in accordance with the selection is stored in the memory 51. The CPU 100 determines a next selection based on the signal-to-noise ratio (S/N) corresponding to the previous selection stored in the memory 51 and the signal-to-noise ratio (S/N) corresponding to the current selection. In other words, in the above example, based on the result of step S225 in FIG. 23A, the process proceeds to step S226 in FIG. 23A to select the transmission/reception antenna pair (TX1, RX1) or the process proceeds to step S230 in FIG. 23C to select the antenna pair (TX4, RX4). Therefore, according to the operation flow of FIG. 23A to 23C, the transmission/reception antenna pairs (TX1, RX1), (TX2, RX2), (TX3, RX3), or (TX4, RX4) suitable for use can be searched for from a plurality of antenna elements according to the situation of the signal-to-noise ratio (S/N).

In the above operation flow, for the sake of simplicity, in step S225 in FIG. 23A, it is assumed that S/N_{(TX3,RX3)} is smaller than S/N_{(TX2,RX2)} in both of the pulse wave signals PS1 and PS2, or S/N_{(TX3,RX3)} is larger than S/N_{(TX2,RX2)} in both of the wave signals PS1 and PS2. However, this example does not set any limitation. For example, the selection of the transmission/reception antenna pair in the first set of transmission/reception antenna pairs (41, 42) and the selection of the transmission/reception antenna pair in the second set of transmission/reception antenna pairs (44, 43) are performed independently of each other, and in a case where S/N_{(TX3,RX3)} is smaller than S/N_{(TX2,RX2)} in the pulse wave signal PS1 and S/N_{(TX3,RX3)} is larger than S/N_{(TX2,RX2)} in the pulse wave signal PS2, the selection of the next transmission/reception antenna pair in the first transmission/reception antenna pair (41, 42) may be different from the selection of the next transmission/reception antenna pair in the second transmission/reception antenna pair (44, 43). Or contrary to the above, also in a case where S/N_{(TX3,RX3)} is larger than S/N_{(TX2,RX2)} in the pulse wave signal PS1 and S/N_{(TX3,RX3)} is larger than S/N_{(TX2,RX2)} in the pulse wave signal PS2, the selection of the next transmission/reception antenna pair in the first set of transmission/reception antenna pairs (41, 42) and the selection of next transmission/reception antenna in the second set of transmission/reception antenna pairs (44, 43) may be different from each other, in a similar manner. With this configuration, in a case where the belt 20 is worn to the left wrist 90, and the belt 20 obliquely intersects the radial artery 91 so that the transmission/reception antenna group 40E is obliquely displaced in the paper plane of FIG. 3 for example, a transmission/reception antenna pairs suitable for use can be respectively searched in the first set of transmission/reception antenna pairs (41, 42) and the second set of transmission/reception antenna pairs (44, 43) according to the situation of the signal-to-noise ratio (S/N).

Further, in the above example, the next selection is determined based on the signal-to-noise ratio S/N_{(TX3,RX3)} according to the previous selection as “past” and the signal-to-noise ratio S/N_{(TX2,RX2)} according to the current selection. However, this example does not set any limitation. A signal-to-noise ratio (S/N) corresponding to a plurality of selections may be used such as the last selection and the selection before the last selection as the “past.” Thereby, the search accuracy can be improved.

Here, in the operation flow of FIGS. 23A to 23C, the CPU 100 determines a next “selection” based on the signal-to-noise ratio (S/N) corresponding to the previous selection stored in the memory 51 and the signal-to-noise ratio (S/N) corresponding to the current selection. However, the dynamic search according to the condition of the signal-to-noise ratio (S/N) is not limited to “selection”, and can also be applied to the case of “weighting.” For example, each time the CPU 100 switches the weight once, the signal-to-noise ratio (S/N) of the signal received in accordance with the weighting may be stored in the memory 51. Then, the CPU 100 may determine a next weighting based on the signal-to-noise ratio (S/N) corresponding to the previous weighting stored in the memory 51 and the signal-to-noise ratio (S/N) corresponding to the current weighting. In this case, it is possible to search for a weight suitable for use among a plurality of antenna elements according to the situation of the signal-to-noise ratio (S/N).

### Modification

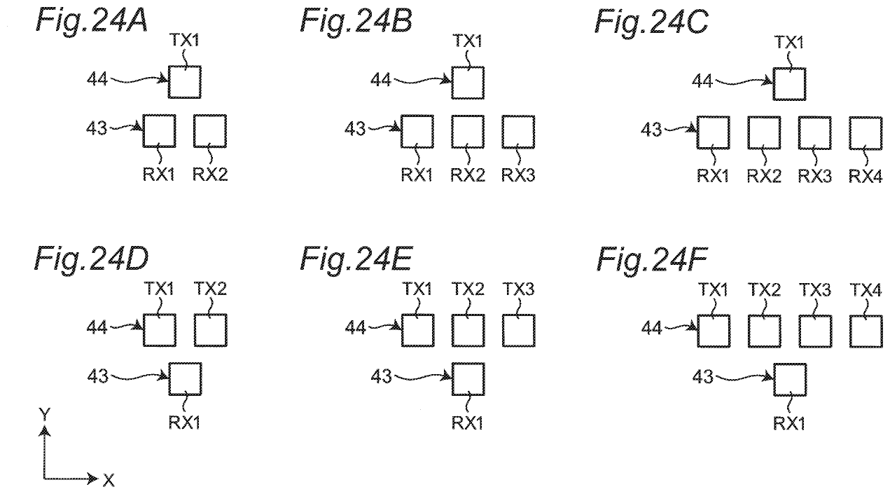

In the above-described embodiment, for example, as illustrated in FIG. 3, the second set of transmission/reception antennas pairs (44, 43) in the transmission/reception antenna group 40E includes four transmission antennas TX1, TX2, TX3, and TX4 arranged along the longitudinal direction X of the belt 20 and four reception antennas RX1, RX2, RX3, and RX4 arranged along the longitudinal direction X. The first set of transmission/reception antenna pairs (41, 42) is similarly configured. However, this example does not set any limitation. For example, as illustrated in FIG. 24A, the second set of transmission/reception antenna pairs (44, 43) may include one transmission/reception antenna TX1 and two reception antennas RX1 and RX2 arranged along the longitudinal direction X. These can be used as two transmission/reception antenna pairs (TX1, RX1) and (TX1, RX2). Further, as illustrated in FIG. 24B, the second set of transmission/reception antenna pairs (44, 43) may include one transmission/reception antenna TX1 and three reception antennas RX1, RX2, and RX3 arranged along the longitudinal direction X. These can be used as three transmission/reception antenna pairs (TX1, RX1), (TX1, RX2), and (TX1, RX3). Further, as illustrated in FIG. 24C, the second set of transmission/reception antenna pairs (44, 43) may include one transmission/reception antenna TX1 and four reception antennas RX1, RX2, RX3, and RX4 arranged along the longitudinal direction X. These can be used as four transmission/reception antenna pairs (TX1, RX1), (TX1, RX2), (TX1, RX3) and (TX1, RX4). Further, as illustrated in FIG. 24D, the second set of transmission/reception antenna pairs (44, 43) may include two transmission antennas TX1 and TX2 arranged along the longitudinal direction X and one reception antenna RX1. These can be used as two transmission/reception antenna pairs (TX1, RX1) and (TX2, RX1). Further, as illustrated in FIG. 24E, the second set of transmission/reception antenna pairs (44, 43) may include three transmission antennas TX1, TX2 and TX3 arranged along the longitudinal direction X and one reception antenna RX1. These can be used as three transmission/reception antenna pairs (TX1, RX1), (TX2, RX1), and (TX3, RX1). Further, as illustrated in FIG. 24F, the second set of transmission/reception antenna pairs (44, 43) may include four transmission antennas TX1, TX2, TX3, and TX4 arranged along the longitudinal direction X and one reception antenna RX1. These can be used as four transmission/reception antenna pairs (TX1, RX1), (TX2, RX1), (TX3, RX1), and (TX4, RX1). The same applies to the first set of transmission/reception antenna pairs (41, 42).

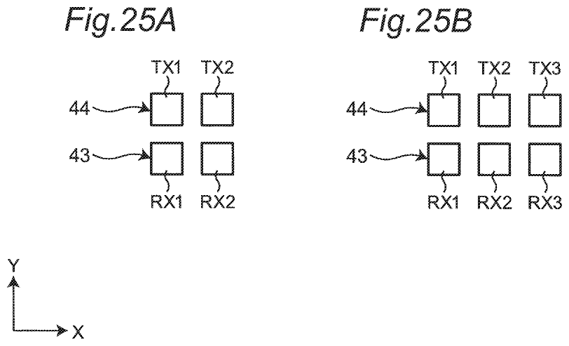

Further, even when the number of transmission antennas arranged along the longitudinal direction X and the number of reception antennas arranged along the longitudinal direction X are the same, and the transmission antenna and the reception antenna arranged along the width direction Y are used as a pair of transmission/reception antennas, as illustrated in FIG. 25A, the second set of transmission/reception antenna pairs (44, 43) may be configured with only two transmission antennas TX1 and TX2 arranged along the longitudinal direction X and two reception antennas RX1 and RX2 arranged along the longitudinal direction X. Further, as illustrated in FIG. 24B, the second set of transmission/reception antenna pairs (44, 43) may be configured with only three transmission/reception antennas TX1, TX2, and TX3 and three reception antennas RX1, RX2, and RX3 arranged along the longitudinal direction X. The same applies to the first set of transmission/reception antenna pairs (41, 42).

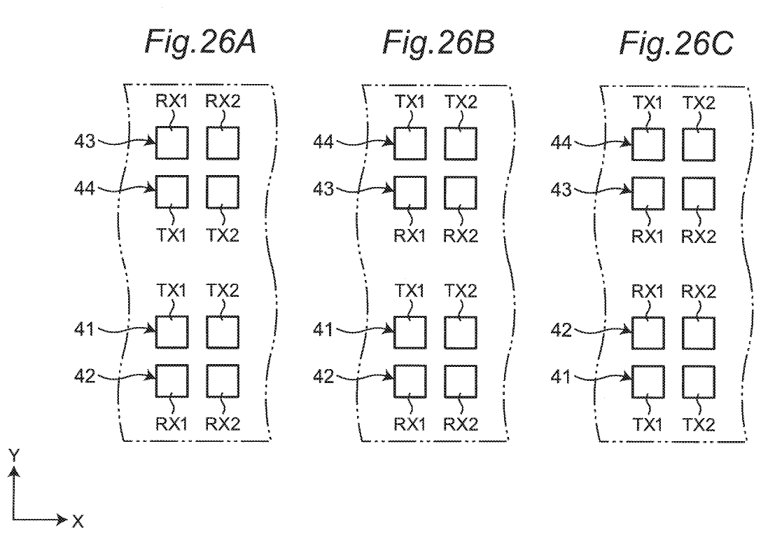

In the example of FIG. 3, as illustrated in a partially enlarged view in FIG. 26C, the transmission antenna arrays 41 and 44 are arranged on opposite sides of the width direction Y in the range where the transmission/reception antenna group 40E is provided, and the reception antenna arrays 42 and 43 are arranged between the transmission antenna arrays 41 and 44. However, this example does not set any limitation. As illustrated in FIG. 26A, the reception antenna arrays 42 and 43 may be arranged on opposite sides within the range where the transmission/reception antenna group 40E is provided, and the transmission antenna arrays 41 and 44 may be arranged between these reception antenna arrays 42 and 43. In this arrangement, the reception antenna array 42 is closer to the transmission antenna array 41 than the reception antenna array 43 with respect to the width direction Y. Further, with respect to the width direction Y, the reception antenna array 43 is closer to the transmission antenna array 44 than the reception antenna array 42. Therefore, interference between the first set of transmission/reception antenna pairs (41, 42) and the second set of transmission/reception antenna pairs (44, 43) can be reduced.

When the distance between the first set of transmission/reception antenna pairs (41, 42) and the second set of transmission/reception antenna pairs (44, 43) is sufficiently secured with respect to the width direction Y, as illustrated in 26B, the arrangement of the transmission antenna array 41 and the reception antenna array 42 in the first set of transmission/reception antenna pairs (41, 42) and the arrangement of the transmission antenna array 44 and the reception antenna array 43 in the second set of transmission/reception antenna pairs (44, 43) may be the same (arrangement that overlaps when moved in parallel).

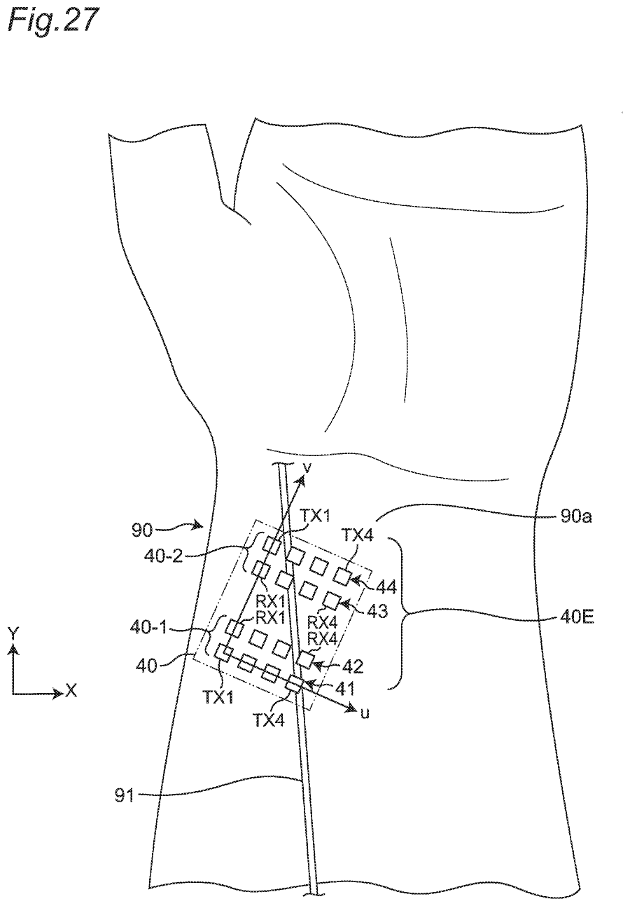

In the above-described embodiment, as illustrated in FIG. 3, along the longitudinal direction X and the width direction Y of the belt 20, the transmission antennas TX1, TX2, . . . and the reception antennas RX1, RX2, . . . as the plurality of antenna elements are arranged spaced apart from each other. However, this example does not set any limitation. As illustrated in FIG. 27, the direction in which the transmission antennas TX1, TX2, . . . and the reception antennas RX1, RX2, . . . as a plurality of antenna elements are arranged may be inclined with respect to the longitudinal direction X and the width direction Y of the belt 20. In this example, four transmission antennas TX1, TX2, TX3, and TX4 forming the first set of transmission antenna array 41 are arranged spaced apart from each other, and four reception antennas RX1, RX2, RX3, and RX4 forming the reception antenna array 42 are arranged spaced apart from each other along one direction u inclined with respect to the longitudinal direction X and the width direction Y in the plane of the belt 20. The four transmission antennas TX1, TX2, TX3, and TX4 and the four reception antennas RX1, RX2, RX3, and RX4 are arranged spaced apart from each other along a direction v orthogonal to the one direction u. The second set of transmission/reception antenna pairs (43, 44) is also arranged in a similar manner. In this manner, even when the direction u and the direction v in which the transmission antennas TX1, TX2, . . . and the reception antennas RX1, RX2, . . . as the plurality of antenna elements are arranged along are inclined with respect to the longitudinal direction X and width direction Y of the belt 20, for example, an appropriate transmission/reception antenna pair can be selected or weighted appropriately. Thereby, the signal-to-noise ratio of the received signal can be increased. As a result, biological information can be measured with high accuracy. Note that the inclination in the direction u and the direction v with respect to the longitudinal direction X and the width direction Y does not have to be the inclination in the direction rotated clockwise as illustrated in FIG. 27, and the inclination may be in the direction rotated counterclockwise.

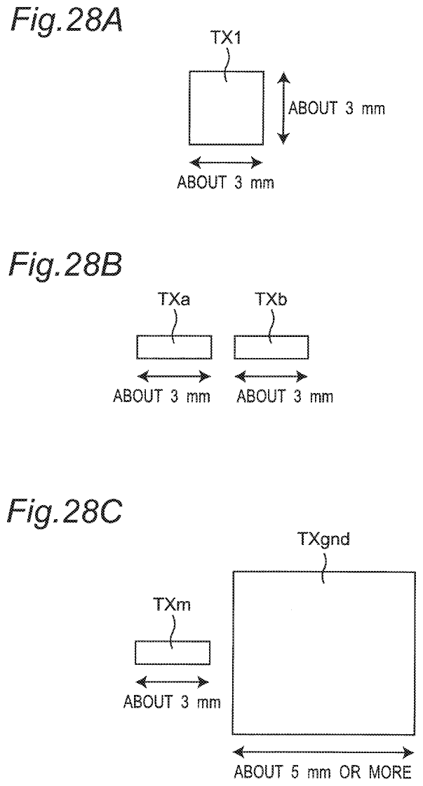

Further, in the above-described embodiment, as illustrated in an enlarged view in FIG. 28A, each antenna element (the transmission antenna TX1 is illustrated in FIG. 28A) is an antenna (patch antenna) that has a square pattern shape of about 3 mm in length and width with respect to the surface direction so as to emit or receive a radio wave having a frequency of 24 GHz band. However, this example does not set any limitation. As illustrated in FIG. 28B, each antenna element may be a dipole antenna in which two portions TXa and TXb each having a length of about 3 mm are arranged in a straight line. As illustrated in FIG. 28C, each antenna element may be a monopole antenna including a rectangular ground portion TXgnd having a length and width of about 5 mm or more and a monopole portion TXm having a length of about 3 mm.

In the above-described embodiment, the antenna element used as the transmission antenna and the antenna element used as the reception antenna are spatially separated from each other. However, this example does not set any limitation. The antenna element constituting the antenna device for biological measurement may be used as a single transmission/reception antenna spatially via a known circulator for the emission and reception of radio waves.

In the above-described embodiment, the sphygmomanometer 1 is to be worn to the left wrist 90 as a measurement target site. However, this example does not set any limitation. The measurement target site only needs to have an artery passing therethrough, and may be a right wrist, an upper limb such as an upper arm other than the wrist, or a lower limb such as an ankle or thigh.

In the above-described embodiment, the CPU 100 mounted on the sphygmomanometer 1 serves as a pulse wave detection unit, first and second blood pressure calculation units to measure blood pressure by the oscillometric method (the operation flow in FIG. 8B) and measure (estimate) blood pressure based on a PTT (the operation flow in FIG. 10). However, this example does not set any limitation. For example, a substantial computer device such as a smartphone provided outside the sphygmomanometer 1 may serve as a pulse wave detection unit and first and second blood pressure calculation units to cause the sphygmomanometer 1 via the network 900 to measure blood pressure by the oscillometric method (the operation flow in FIG. 8B) and measure (estimate) blood pressure based on the PTT (the operation flow in FIG. 10). In this case, the user performs an operation such as an instruction to start or stop blood pressure measurement using the operation unit (touch panel, keyboard, mouse, etc.) of the computer device to cause the display unit (organic EL display, LCD, etc.) of the computer device to display information related to blood pressure measurement such as blood pressure measurement results and other information. In that case, in the sphygmomanometer 1, the display unit 50 and the operation unit 52 may be omitted.

In the above-described embodiment, the sphygmomanometer 1 measures the pulse wave signal, the pulse wave transit time, and the blood pressure as biological information, but this does not set any limitation. Various other biological information such as the pulse rate may be measured.

Moreover, according to the present invention, an apparatus may be configured with the antenna device for biological measurement, pulse wave measuring device, and blood pressure measuring device and further configured with a functional part which performs another function. According to this apparatus, biological information can be measured with high accuracy, a pulse wave signal can be acquired with high accuracy as biological information, or a blood pressure value can be calculated (estimated) with high accuracy. In addition, this apparatus can perform various functions.

In order to achieve the above object, in a first aspect, an antenna device for biological measurement of the present disclosure is a device that emits radio waves toward a measurement target site of a living body or receives radio waves from the measurement target site to measure biological information, the device comprising:

a belt worn as surrounding a measurement target site of a living body;

a transmission/reception antenna group provided to the belt and including a plurality of antenna elements arranged, in an area where the belt is spread in a strip-like manner, being spaced apart from each other in one direction or two orthogonal directions;

a transmission circuit configured to emit a radio wave toward the measurement target site using any one of antenna elements included in the transmission/reception antenna group as a transmission antenna, in a wearing state where the belt is worn as surrounding an outer surface of the measurement target site;

a reception circuit configured to receive a radio wave reflected from the measurement target site using any one of antenna element included in the transmission/reception antenna group as a reception antenna; and

an antenna control unit configured to weight a transmission/reception antenna pair formed of the transmission antenna and the reception antenna among the plurality of antenna elements based on an output of the reception circuit.

In the present specification, the “measurement target site” may be a trunk in addition to a rod-shaped site such as an upper limb (wrist, upper arm, or the like) or a lower limb (ankle, or the like).

Further, the “outer surface” of the measurement target site refers to a surface exposed to the outside. For example, in a case the measurement target site is a wrist, an outer surface refers to the outer peripheral surface of the wrist or a part thereof (for example, the palmar side surface corresponding to the palm side portion of the outer peripheral surface in the circumferential direction).

Further, the “belt” refers to a band-like member for surrounding the measurement target site, and another term such as “band” may be used.

Further, each “antenna element” refers to an element used as a transmission antenna or a reception antenna, or as a transmission/reception shared antenna via a known circulator.

In addition, the “surface” of the belt spreads in a band-like shape does not indicate whether it is an inner peripheral surface or an outer peripheral surface. The “one direction” in the plane typically refers to the “longitudinal direction” or “width direction” of the belt, but may be a direction obliquely inclined with respect to the “longitudinal direction” or “width direction.” In addition, the “two orthogonal directions” in the plane along the measurement target site of the belt refers to two directions, for example, the “one direction” and a direction orthogonal to the “one direction.” The “longitudinal direction” of the belt corresponds to the circumferential direction of the measurement target site in a wearing state to the measurement target site. The “width direction” of the belt refers to a direction crossing the “longitudinal direction” of the belt.

In addition, to “weight” the transmission/reception antenna pair refers to, for example, that a weight of an antenna element used as a certain transmission/reception antenna pair is set relatively heavy among a plurality of antenna elements, and the weights of other antenna elements are set relatively light.

In this specification, “weight” does not refer to physical weight, but refers to a relative degree (large or small) of usage of each element in a case where a plurality of elements (antenna elements) are used in parallel at the same time.

The antenna device for biological measurement according to the present disclosure is worn to the measurement target site by a user (including a subject person, and the same applies hereinafter) by putting the belt around an outer surface of the measurement target site. In this wearing state, the transmission circuit emits radio waves toward the measurement target site using any one of the antenna elements included in the transmission/reception antenna group as a transmission antenna, and the reception circuit receives radio waves reflected by the measurement target site using any one of the antenna elements included in the transmission/reception antenna group as a reception antenna. Based on an output from the reception circuit, the antenna control unit performs a process of weighting the transmission/reception antenna pair formed by the transmission antenna and the reception antenna among the plurality of antenna elements. With this configuration, via the transmission/reception antenna pair weighted by the antenna control unit, the transmission circuit emits radio waves toward the measurement target site and the reception circuit receives radio waves reflected by the measurement target site. Therefore, even in a case where a position displacement of the transmission/reception antenna group occurs with respect to the measurement target site, the transmission/reception antenna pair is appropriately weighted among the plurality of antenna elements. Thereby, the signal-to-noise ratio of the received signal can be increased. As a result, biological information can be measured with high accuracy.

In the antenna device for biological measurement of one embodiment, the antenna control unit acquires a signal-to-noise ratio of received signal and weights the transmission/reception antenna pair among the plurality of antenna elements so that the acquired signal-to-noise ratio becomes larger than α predetermined reference value.

In the antenna device for biological measurement of the one embodiment, the antenna control unit can make the signal-to-noise ratio of received signal larger than the reference value. Therefore, biological information can be reliably obtained from the measurement target site. Also, for example, in a case where a certain signal-to-noise ratio obtained is larger than the reference value in the process of weighting the transmission/reception antenna pairs among the plurality of antenna elements, the switching can be stopped at that time to complete the process. Therefore, weighting process by the antenna control unit can be completed more quickly than α case where all the switching operations are tried.

In the antenna device for biological measurement of one embodiment, the plurality of antenna elements are arranged spaced apart from each other within a predetermined area along a longitudinal direction of the belt.

Here, the “predetermined area” refers to an area on the belt corresponding to a portion of the measurement target site where biological information is acquired. For example, in a case where the measurement target site is a wrist and a pulse wave is measured as biological information, the “predetermined area” is set along the longitudinal direction of the belt so as to correspond to the portion of the wrist including the radial artery.

In the antenna device for biological measurement of the one embodiment, even when the belt is worn to the measurement target site and the transmission/reception antenna group is displaced with respect to the measurement target site in the circumferential direction (corresponding to the longitudinal direction of the belt), some of the plurality of antenna elements may be close to a portion of the measurement target site where biological information is acquired. Therefore, when the antenna control unit performs the weighting process, a transmission/reception antenna pair suitable for use (or a weight suitable for use) is determined among the plurality of antenna elements. Therefore, the signal-to-noise ratio of the received signal can be increased, and as a result, biological information can be measured with high accuracy.

In the antenna device for biological measurement of one embodiment, the plurality of antenna elements are arranged spaced apart from each other along the longitudinal direction of the belt and arranged spaced apart so that the transmission/reception antenna pairs are formed along a width direction of the belt.

In the antenna device for biological measurement of the one embodiment, even when the belt is worn to the measurement target site and the transmission/reception antenna group is displaced with respect to the measurement target site in the circumferential direction (corresponding to the longitudinal direction of the belt), some of the transmission/reception antenna pairs of the plurality of transmission/reception antenna pairs may be close to the portion of the measurement target site where biological information is acquired, in the longitudinal direction of the belt. Therefore, when the antenna control unit performs the weighting process, among the plurality of antenna elements, the transmission/reception antenna pair suitable for use (or weighting suitable for use for the plurality of transmission/reception antenna pairs) in the longitudinal direction of the belt can be determined. Therefore, the signal-to-noise ratio of the received signal can be increased, and as a result, biological information can be measured with high accuracy. Further, since the plurality of antenna elements are arranged spaced apart from each other so as to form a transmission/reception antenna pair along the width direction of the belt, transmission and reception are performed simultaneously by the transmission/reception antenna pair without using a circulator.

In a second aspect, an antenna device for biological measurement according to the present disclosure is an antenna device for biological measurement that measures biological information, the device comprising:

a belt worn as surrounding a measurement target site of a living body;

a transmission/reception antenna group provided to the belt and including a plurality of antenna elements arranged, in an area where the belt is spread in a strip-like manner, being spaced apart from each other in one direction or two orthogonal directions;

a transmission circuit configured to emit a radio wave toward the measurement target site using any one of antenna elements included in the transmission/reception antenna group as a transmission antenna, in a wearing state where the belt is worn as surrounding an outer surface of the measurement target site;

a reception circuit configured to receive a radio wave reflected from the measurement target site using any one of antenna element included in the transmission/reception antenna group as a reception antenna;

an antenna control unit configured to select or to weight by switching a transmission/reception antenna pair formed of the transmission antenna and the reception antenna among the plurality of antenna elements based on an output of the reception circuit; and

a storage unit configured to store a signal-to-noise ratio of received signal corresponding to selection or weighting every time the antenna control unit switches the selection or weighting once,

wherein the antenna control unit determines a next selection or weighting based on a signal-to-noise ratio corresponding to past selection or weighting, which is stored in the storage unit, and a signal-to-noise ratio corresponding to the current selection or weighting.

In the present specification, “by switching” is not limited to switching both a transmission antenna and a reception antenna among a plurality of antenna elements and includes, for example, a case where a certain antenna element is fixedly used as the transmission antenna and the reception antenna is switched among a plurality of antenna elements, and a case where a certain antenna element is fixedly used as the reception antenna and the transmission antenna is switched among a plurality of antenna elements.

Further, to “select” a transmission/reception antenna pair refers to, for example, selecting antenna elements used as a certain transmission/reception antenna pair among a plurality of antenna elements and deselecting other antenna elements.

In the antenna device for biological measurement of the one embodiment, a transmission/reception antenna pair suitable for use can be searched for from the plurality of antenna elements according to the situation of the signal-to-noise ratio (S/N).

In a third aspect, a pulse wave measuring device according to present disclosure is a pulse wave measuring device that measures a pulse wave at a measurement target site of a living body, the device comprising the antenna device for biological measurement of the second aspect, wherein

the area where the transmission/reception antenna group is provided is placed corresponding to an artery that passes through the measurement target site in the wearing state where the belt is worn as surrounding the outer surface of the measurement target site, and

in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit selects by switching or weights the transmission/reception antenna pair formed of the transmission antenna and the reception antenna among the plurality of antenna elements based on an output from the reception circuit,

further comprising a pulse wave detection unit configured to acquire a pulse wave signal indicating a pulse wave at the artery passing through the measurement target site based on the output from the reception circuit received via the selected or weighted transmission/reception antenna pair.

In the pulse wave measuring device according to the present disclosure, the antenna control unit selects or weights the transmission/reception antenna pair among the plurality of antenna elements. Therefore, even in a case where a position displacement of the transmission/reception antenna group occurs with respect to the measurement target site, for example, an appropriate transmission/reception antenna pair is selected, or the transmission/reception antenna pair is appropriately weighted among the plurality of antenna elements. Thereby, the signal-to-noise ratio of the received signal can be increased. As a result, the pulse wave signal as the biological information can be measured with high accuracy.

In a fourth aspect, a blood pressure measuring device according to the present disclosure is a blood pressure measuring device that measures blood pressure at a measurement target site of a living body, the device comprising two sets of pulse wave measuring devices of the third aspect,

wherein the belts of the two sets are integrally formed,

the transmission/reception antenna group of the two sets are arranged spaced apart from each other in a width direction of the belt,

in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,

in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit selects by switching or weights the transmission/reception antenna pair formed of the transmission antenna and the reception antenna among the plurality of antenna elements based on an output from the reception circuit, and

respectively in the two sets, the pulse wave detection unit acquires the pulse wave signal indicating the pulse wave at the artery passing through the measuring site based on the output from the reception circuit received via the selected or weighted transmission/reception antenna pair,

further comprising:

a time difference acquisition unit configured to acquire a time difference between the pulse wave signals respectively acquired by the pulse wave detection unit of the two sets as a pulse wave transit time; and

a first blood pressure calculation unit configured to calculate blood pressure value based on the pulse wave transit time acquired by the time difference acquisition unit using a predetermined correspondence equation between the pulse wave transit time and the blood pressure.

In the blood pressure measuring device according to the present disclosure, respectively in the two sets, the antenna control unit selects or weights the transmission/reception antenna pair among the plurality of antenna elements. Therefore, even in a case where a position displacement of the transmission/reception antenna group of the two sets occurs with respect to the measurement target site, respectively in the two sets, for example, an appropriate transmission/reception antenna pair is selected, or the transmission/reception antenna pair is appropriately weighted among the plurality of antenna elements. Therefore, the signal-to-noise ratio of the received signal can be increased, and the pulse wave detection unit can accurately acquire a pulse wave signal as biological information. As a result, the time difference acquisition unit can acquire the pulse wave transit time with high accuracy, and thus the first blood pressure calculation unit can calculate (estimate) the blood pressure value with high accuracy.

Returning to the first aspect, in the antenna device for biological measurement of one embodiment, the antenna control unit searches for weighting with which a signal-to-noise ratio of received signal becomes large, by setting an antenna element in the plurality of antenna elements with a relatively heavy weight as sequentially switching from an antenna element arranged at an end in one side to an antenna element arranged at an end on other side in the area where the transmission/reception antenna group is provided in the longitudinal direction of the belt.

In this specification, setting “relatively heavy weight” refers to setting a weight to a certain antenna element in the plurality of antenna elements relatively heavy while reducing the weight for antenna elements other than the above antenna element. Further, “sequentially switching from an element arranged at an end on one side to an element arranged at an end on other side” refers to sequentially switching from an element arranged at one end (which is referred to as a first element), to an element adjacent to the first element on the other side (which is referred to as a second element), an element adjacent to the second element on the other side (which is referred to as a third element), an element adjacent to the third element on the other side (which is referred to as a fourth element), and so on.

In the antenna device for biological measurement of the one embodiment, a weight suitable for use is reliably determined from the plurality of antenna elements.

In the antenna device for biological measurement of one embodiment, the antenna control unit searches for weighting with which a signal-to-noise ratio of received signal becomes large, by setting an antenna element in the plurality of antenna elements with a relatively heavy weight as sequentially switching from an antenna element arranged at a central portion to an antenna element arranged at ends in opposite sides alternately in the area where the transmission/reception antenna group is provided in the longitudinal direction of the belt.

Here, “sequentially switching from an element arranged at a central portion to an element arranged at ends in opposite sides alternately” refers to sequentially switching from an element arranged at the central portion (which is referred to as a first element), to an element adjacent to the first element on one side (which is referred to as a second element), an element adjacent to the first element on the other side (which is referred to as a third element), an element adjacent to the second element on the one side (which is referred to as a fourth element), an element adjacent to the third element on the other side (which is referred to as a fifth element), and the like.

When the belt is worn to the measurement target site, the amount of position displacement of the transmission/reception antenna group with respect to the measurement target site is assumed to indicate frequency of normal distribution in a statistical viewpoint centered on the portion of the measurement target site where biological information is acquired. Therefore, in the antenna device for biological measurement of the one embodiment, the antenna control unit searches for weighting with which a signal-to-noise ratio of received signal becomes large, by setting an antenna element in the plurality of antenna elements with a relatively heavy weight as sequentially switching from an antenna element arranged at a central portion to an antenna element arranged at ends in opposite sides alternately in the area where the transmission/reception antenna group is provided in the longitudinal direction of the belt. With this configuration, a weight suitable for use can be reliably and quickly determined from the plurality of antenna elements.

In the antenna device for biological measurement of one embodiment, the transmission/reception antenna group includes the plurality of antenna elements in M rows and N columns arrangement, where M and N are natural numbers of 2 or more, respectively, and includes the antenna elements arranged to form two transmission antennas along the longitudinal direction of the belt and the antenna elements arranged to form two reception antennas along the longitudinal direction of the belt as two rows and two columns arrangement in the M rows and N columns, and

the antenna control unit searches for weighting with which a signal-to-noise ratio of received signal becomes large, by switching
a first setting that sets a first transmission antenna and a first reception antenna, in the two transmission antennas and the two reception antennas, arranged at one side in the longitudinal direction of the belt with a relatively heavy weight,
a second setting that sets a second transmission antenna and a second reception antenna, in the two transmission antennas and the two reception antennas, arranged at other side in the longitudinal direction of the belt with a relatively heavy weight,
a third setting that sets the first transmission antenna and the second reception antenna with a relatively heavy weight, and
a fourth setting that sets the second transmission antenna and the first reception antenna with a relatively heavy weight.

In the antenna device for biological measurement of the one embodiment, the antenna control unit performs switching between a first setting for setting a relatively heavy weight to a first transmission antenna and a first reception antenna arranged at one side with respect to the longitudinal direction of the belt in the two transmission antennas and the two reception antennas, and a second setting for setting a relatively heavy weight to a second transmission antenna and a second reception antenna arranged at other side in the longitudinal direction of the belt in the the two transmission antennas and the two reception antennas. With this configuration, even when the belt is worn to the measurement target site and a position displacement of the transmission/reception antenna group occurs in the circumferential direction with respect to the measurement target site, in the first and second sets of transmission/reception antenna pairs, one of the sets of transmission/reception antenna pairs can increase the signal-to-noise ratio of the received signal, and as a result, the biological information can be measured with high accuracy. Further, the antenna control unit performs switching between a third setting for setting a relatively heavy weight to the first transmission antenna and the second reception antenna, and a fourth setting for setting a relatively heavy weight to the second transmission antenna and the first reception antenna. With this configuration, even when the belt is worn to the measurement target site and the belt intersects obliquely with respect to the artery passing through the measurement target site so that the transmission/reception antenna group is obliquely displaced, the signal-to-noise ratio of the received signal can be increased by any one of the third and fourth transmission/reception antenna pairs, and as a result, the biological information can be accurately measured.

Here, a matrix formed by the transmission/reception antenna group includes the plurality of antenna elements in an arrangement of M rows and N columns, where M and N are natural numbers of 2 or more, respectively. For example, if M=N=2, the matrix formed by the transmission/reception antenna group is only two rows and two columns. However, the matrix formed by the transmission/reception antenna group is not limited to two rows and two columns, and may be in, for example, a multiple rows and multiple columns with M≥3 and N≥3. In this case, the antenna control unit performs the switching described above for one or more sets of two rows and two columns of antenna elements included in the multiple rows and multiple columns. Also, the two rows and two columns of antenna elements to be controlled do not need to be adjacent to each other, and another antenna element may be arranged between these antenna elements.

In the antenna device for biological measurement of one embodiment, every time the weighting is switched once, the antenna control unit shifts a relative phase of radio waves emitted by the transmission antenna formed by the plurality of antenna elements and/or a relative phase of signals received by the reception antenna formed by the plurality of antenna elements, thereby controlling to increase a signal-to-noise ratio of a combined signal obtained by combining the signals.

In the weighting method, it is still need to adjust the relative phase shift among the radio waves emitted by the transmission antennas formed of the plurality of antenna elements or the relative phase shift among the signals respectively received by the reception antennas formed of the plurality of antenna elements. Therefore, in the antenna device for biological measurement of the one embodiment, every time the weighting is switched once, the antenna control unit shifts a relative phase of radio waves emitted by the transmission antenna formed by the plurality of antenna elements and/or a relative phase of signals received by the reception antenna formed by the plurality of antenna elements, thereby controlling to increase a signal-to-noise ratio of a combined signal obtained by combining the signals. Therefore, the phase shift among the received signals is adjusted and the signal-to-noise ratio is further improved.

In the antenna device for biological measurement of one embodiment, every time the weighting is switched once, the antenna control unit changes a relative weight of radio waves emitted by the plurality of transmission antennas and a relative weight of signals respectively received by the plurality of reception antennas, thereby controlling to increase a signal-to-noise ratio of a combined signal obtained by combining the signals.

In the weighting method, it is still need to adjust the relative weighting among the radio waves emitted by the transmission antennas formed of the plurality of antenna elements or the relative weight among the signals respectively received by the reception antennas formed of the plurality of antenna elements. Therefore, in the antenna device for biological measurement of the one embodiment, every time the weighting is switched once, the antenna control unit changes a relative weight of radio waves emitted by the plurality of transmission antennas and a relative weight of signals respectively received by the plurality of reception antennas, thereby controlling to increase a signal-to-noise ratio of a combined signal obtained by combining the signals. Therefore, the relative weighting among the received signals is adjusted and the signal-to-noise ratio is further improved.

In a fifth aspect, a pulse wave measuring device according to the present disclosure is a device that measures a pulse wave at a measurement target site of a living body, the device comprising the antenna device for biological measurement of the first aspect, wherein

the area where the transmission/reception antenna group is provided is placed corresponding to an artery that passes through the measurement target site in the wearing state where the belt is worn as surrounding the outer surface of the measurement target site, and

in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit weights the transmission/reception antenna pair formed of the transmission antenna and the reception antenna among the plurality of antenna elements based on an output from the reception circuit,

further comprising a pulse wave detection unit configured to acquire a pulse wave signal indicating a pulse wave at the artery passing through the measurement target site based on the output from the reception circuit received via the weighted transmission/reception antenna pair.

In the pulse wave measuring device according to the present disclosure, the antenna control unit weights the transmission/reception antenna pair among the plurality of antenna elements. Therefore, even in a case where a position displacement of the transmission/reception antenna group occurs with respect to the measurement target site, the transmission/reception antenna pair is appropriately weighted among the plurality of antenna elements. Thereby, the signal-to-noise ratio of the received signal can be increased. As a result, the pulse wave signal as the biological information can be measured with high accuracy.

In a sixth aspect, a blood pressure measuring device according to the present disclosure is a device that measures blood pressure at a measurement target site of a living body, the device comprising two sets of the pulse wave measuring devices of the fifth aspect,

wherein the belts of the two sets are integrally formed,

the transmission/reception antenna group of the two sets are arranged spaced apart from each other in a width direction of the belt,

in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,

in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit weights the transmission/reception antenna pair formed of the transmission antenna and the reception antenna among the plurality of antenna elements based on an output from the reception circuit, and

respectively in the two sets, the pulse wave detection unit acquires the pulse wave signal indicating the pulse wave at the artery passing through the measuring site based on the output from the reception circuit received via the weighted transmission/reception antenna pair,

further comprising:

a time difference acquisition unit configured to acquire a time difference between the pulse wave signals respectively acquired by the pulse wave detection unit of the two sets as a pulse wave transit time; and

a first blood pressure calculation unit configured to calculate blood pressure value based on the pulse wave transit time acquired by the time difference acquisition unit using a predetermined correspondence equation between the pulse wave transit time and the blood pressure.

In the blood pressure measuring device according to the present disclosure, respectively in the two sets, the antenna control unit weights the transmission/reception antenna pair among the plurality of antenna elements. Therefore, even in a case where a position displacement of the transmission/reception antenna group of the two sets occurs with respect to the measurement target site, respectively in the two sets, the transmission/reception antenna pair is appropriately weighted among the plurality of antenna elements. Therefore, the signal-to-noise ratio of the received signal can be increased, and the pulse wave detection unit can accurately acquire a pulse wave signal as biological information. As a result, the time difference acquisition unit can acquire the pulse wave transit time with high accuracy, and thus the first blood pressure calculation unit can calculate (estimate) the blood pressure value with high accuracy.

In the blood pressure measuring device of one embodiment, a fluid bag for pressing the measurement target site is provided to the belt, and the blood pressure measuring device further comprises:

a pressure control unit configured to control pressure by supplying air in the fluid bag; and

a second blood pressure calculation unit configured to calculate blood pressure by an oscillometric method based on the pressure in the fluid bag.

In the blood pressure measuring device of the one embodiment, blood pressure measurement (estimation) based on the pulse wave transit time and blood pressure measurement by the oscillometric method can be performed using a common belt. Therefore, user convenience is enhanced.

In a seventh aspect, an apparatus according to the present disclosure comprises the above-described antenna device for biological measurement, the above-described pulse wave measuring device, or the above-described blood pressure measuring device.

The apparatus of the present disclosure includes the above-described antenna device for biological measurement, the above-described pulse wave measuring device, or the above-described blood pressure measuring device, and may include a functional unit that performs other functions. According to this apparatus, biological information can be measured with high accuracy, a pulse wave signal can be acquired with high accuracy as biological information, or a blood pressure value can be calculated (estimated) with high accuracy. In addition, this apparatus can perform various functions.

In an eighth aspect, a biological information measuring method according to the present disclosure is a method that measures biological information using a belt to which a transmission/reception antenna group is provided, wherein

the transmission/reception antenna group includes a plurality of antenna elements arranged spaced apart from each other in a longitudinal direction and/or a width direction of the belt,

the biological information measuring method comprising:

wearing the belt as surrounding an outer surface of a measurement target site of the living body into a wearing state so that the transmission/reception antenna group is placed corresponding to an artery passing through the measurement target site; and

in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit.

According to this biological information measuring method, even in a case where a position displacement of the transmission/reception antenna group occurs with respect to the measurement target site, the transmission/reception antenna pair is appropriately weighted among the plurality of antenna elements. Thereby, the signal-to-noise ratio of the received signal can be increased. As a result, biological information can be measured with high accuracy.

In a ninth aspect, a pulse wave measuring method according to the present disclosure is a method that measures a pulse wave of a measurement target site of a living body using a belt to which a transmission/reception antenna group is provided, wherein

the transmission/reception antenna group includes a plurality of antenna elements arranged spaced apart from each other in a longitudinal direction and/or a width direction of the belt,

the pulse wave measuring method comprising:

wearing the belt as surrounding an outer surface of a measurement target site into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit; and

acquiring a pulse wave signal indicating a pulse wave at the artery passing through the measurement target site based on the output from the reception circuit received via the weighted transmission/reception antenna pair.

According to this pulse wave measuring method, even in a case where a position displacement of the transmission/reception antenna group occurs with respect to the measurement target site, the transmission/reception antenna pair is appropriately weighted among the plurality of antenna elements. Thereby, the signal-to-noise ratio of the received signal can be increased. As a result, the pulse wave as the biological information can be measured with high accuracy.

In a tenth aspect, a blood pressure measuring method according to the present disclosure is a method that measures blood pressure at a measurement target site of a living body using a belt to which two sets of transmission/reception antenna groups are integrally provided, wherein

the two sets of the transmission/reception antenna groups are arranged spaced apart from each other in a width direction of the belt and respectively include a plurality of antenna elements arranged spaced apart from each other in a longitudinal direction and/or the width direction of the belt,

the blood pressure measuring method comprising:

wearing the belt as surrounding an outer surface of the measurement target site into a wearing state so that a first set of transmission/reception antenna group of the two sets is placed corresponding to an upstream portion of an artery passing through the measurement target site and a second set of transmission/reception antenna group is placed corresponding to a downstream portion of the artery;

in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, weighting a transmission/reception antenna pair formed of the transmission antenna and the reception antenna among the plurality of antenna elements based on an output from the reception circuit;

respectively in the two sets, acquiring a pulse wave signal indicating a pulse wave at the artery passing through the measurement target site based on the output from the reception circuit received via the weighted transmission/reception antenna pair;

acquiring a time difference between the pulse wave signals respectively received in the two sets as a pulse wave transit time; and

calculating a blood pressure value based on the acquired pulse wave transit time using a predetermined correspondence equation between the pulse wave transit time and the blood pressure.

According to the blood pressure measuring method, even in a case where a position displacement of the transmission/reception antenna group of the two sets occurs with respect to the measurement target site, respectively in the two sets, the transmission/reception antenna pair is appropriately weighted among the plurality of antenna elements. Therefore, the signal-to-noise ratio of the received signal can be increased, and a pulse wave signal as biological information can be accurately acquired. As a result, the pulse wave transit time can be acquired with high accuracy, and the blood pressure value can be calculated (estimated) with high accuracy.

In an eleven aspect, a biological information measuring method of the present disclosure is a biological information measuring method that measures biological information using a belt to which a transmission/reception antenna group is provided, wherein

the transmission/reception antenna group includes a plurality of antenna elements arranged spaced apart from each other in a longitudinal direction and/or a width direction of the belt,

the biological information measuring method comprising:

wearing the belt as surrounding an outer surface of a measurement target site of a living body into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit,

storing a signal-to-noise ratio of received signal corresponding to selection or weighting in a storage unit every time the selection or weighting is switched once, and

determining a next selection or weighting based on a signal-to-noise ratio corresponding to past selection or weighting stored in the storage unit and a signal-to-noise ratio corresponding to current selection or weighting.

In the biological information measuring method according to the present disclosure, a transmission/reception antenna pair suitable for use can be searched for from the plurality of antenna elements according to the situation of the signal-to-noise ratio (S/N).

In a twelve aspect, a pulse wave measuring method according to the present disclosure is a pulse wave measuring method that measures a pulse wave at a measurement target site of a living body using a belt to which a transmission/reception antenna group is provided, wherein

the transmission/reception antenna group includes a plurality of antenna elements arranged spaced apart from each other in a longitudinal direction and/or a width direction of the belt,

the pulse wave measuring method comprising:

wearing the belt as surrounding an outer surface of the measurement target site into a wearing state so that the transmission/reception antenna group is placed corresponding to an artery passing through the measurement target site;

in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;

storing a signal-to-noise ratio of received signal corresponding to selection or weighting in a storage unit every time the selection or weighting is switched once;

determing a next selection or weighting based on a signal-to-noise ratio corresponding to past selection or weighting stored in the storage unit and a signal-to-noise ratio corresponding to current selection or weighting; and

acquiring a pulse wave signal indicating a pulse wave at the artery passing through the measurement target site based on the output from the reception circuit received via the selected or weighted transmission/reception antenna pair.

In the pulse wave measuring method according to the present disclosure, even in a case where a position displacement of the transmission/reception antenna group occurs with respect to the measurement target site, for example, an appropriate transmission/reception antenna pair is selected, or the transmission/reception antenna pair is appropriately weighted among the plurality of antenna elements. Thereby, the signal-to-noise ratio of the received signal can be increased. As a result, the pulse wave as the biological information can be measured with high accuracy.

In a thirteen aspect, a blood pressure measuring method according to the present disclosure is a blood pressure measuring method that measures blood pressure at a measurement target site of a living body using a belt to which two sets of transmission/reception antenna groups are integrally provided, wherein

the two sets of the transmission/reception antenna group are arranged spaced apart from each other in a width direction of the belt and respectively include a plurality of antenna elements arranged spaced apart from each other in a longitudinal direction and/or the width direction of the belt,

the blood pressure measuring method comprising:

wearing the belt as surrounding an outer surface of the measurement target site into a wearing state so that a first set of the transmission/reception antenna group of the two sets is placed corresponding to an upstream portion of an artery passing through the measurement target site and a second set of the transmission/reception antenna group is placed corresponding to a downstream portion of the artery;

in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;

storing a signal-to-noise ratio of received signal corresponding to selection or weighting in a storage unit every time the selection or weighting is switched once;

determining a next selection or weighting based on a signal-to-noise ratio corresponding to past selection or weighting stored in the storage unit and a signal-to-noise ratio corresponding to current selection or weighting;

respectively in the two sets, acquiring a pulse wave signal indicating a pulse wave at the artery passing through the measurement target site based on the output from the reception circuit received via the selected or weighted transmission/reception antenna pair;

acquiring a time difference between the pulse wave signals respectively acquired in the two sets as a pulse wave transit time; and

calculating a blood pressure value based on the acquired pulse wave transit time using a predetermined correspondence equation between the pulse wave transit time and the blood pressure.

In the blood pressure measuring method according to the present disclosure, even in a case where a position displacement of the transmission/reception antenna group of the two sets occurs with respect to the measurement target site, respectively in the two sets, for example, an appropriate transmission/reception antenna pair is selected, or the transmission/reception antenna pair is appropriately weighted among the plurality of antenna elements. Therefore, the signal-to-noise ratio of the received signal can be increased, and a pulse wave signal as biological information can be accurately acquired. As a result, the pulse wave transit time can be acquired with high accuracy, and the blood pressure value can be calculated (estimated) with high accuracy.

As is clear from the above, according to the antenna device for biological measurement and the biological information measuring method of the present disclosure, even when the position of the transmission/reception antenna group is displaced with respect to the measurement target site, biological information can be measured with high accuracy. Moreover, according to the pulse wave measuring device and the pulse wave measuring method of the present disclosure, the pulse wave signal as biological information can be obtained with high accuracy. Moreover, according to the blood pressure measuring device and the blood pressure measuring method of the present disclosure, the blood pressure value can be calculated (estimated) with high accuracy. In addition, according to the apparatus of the present disclosure, biological information can be measured with high accuracy, a pulse wave signal as biological information can be acquired with high accuracy, or a blood pressure value can be calculated (estimated) with high accuracy, and other various functions can be executed.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. An antenna device for biological measurement that measures biological information, the device comprising:
a belt worn as surrounding a measurement target site of a living body;
a transmission/reception antenna group provided to the belt and including a plurality of antenna elements arranged, in an area where the belt is spread in a strip-like manner, being spaced apart from each other in one direction or two orthogonal directions;
a transmission circuit configured to emit a radio wave toward the measurement target site using any one of antenna elements included in the transmission/reception antenna group as a transmission antenna, in a wearing state where the belt is worn as surrounding an outer surface of the measurement target site;
a reception circuit configured to receive a radio wave reflected from the measurement target site using any one of antenna element included in the transmission/reception antenna group as a reception antenna; and
an antenna control unit configured to weight a transmission/reception antenna pair formed of the transmission antenna and the reception antenna among the plurality of antenna elements based on an output of the reception circuit.

2. The antenna device for biological measurement according to claim 1, wherein the antenna control unit acquires a signal-to-noise ratio of received signal and weights the transmission/reception antenna pair among the plurality of antenna elements so that the acquired signal-to-noise ratio becomes larger than α predetermined reference value.

3. The antenna device for biological measurement according to claim 1, wherein the plurality of antenna elements are arranged spaced apart from each other within a predetermined area along a longitudinal direction of the belt.

4. The antenna device for biological measurement according to claim 3, wherein the plurality of antenna elements are arranged spaced apart from each other along the longitudinal direction of the belt and arranged spaced apart so that the transmission/reception antenna pairs are formed along a width direction of the belt.

5. An antenna device for biological measurement that measures biological information, the device comprising:
a belt worn as surrounding a measurement target site of a living body;
a transmission/reception antenna group provided to the belt and including a plurality of antenna elements arranged, in an area where the belt is spread in a strip-like manner, being spaced apart from each other in one direction or two orthogonal directions;
a transmission circuit configured to emit a radio wave toward the measurement target site using any one of antenna elements included in the transmission/reception antenna group as a transmission antenna, in a wearing state where the belt is worn as surrounding an outer surface of the measurement target site;
a reception circuit configured to receive a radio wave reflected from the measurement target site using any one of antenna element included in the transmission/reception antenna group as a reception antenna;
an antenna control unit configured to select or to weight by switching a transmission/reception antenna pair formed of the transmission antenna and the reception antenna among the plurality of antenna elements based on an output of the reception circuit; and
a storage unit configured to store a signal-to-noise ratio of received signal corresponding to selection or weighting every time the antenna control unit switches the selection or weighting once,
wherein the antenna control unit determines a next selection or weighting based on a signal-to-noise ratio corresponding to past selection or weighting, which is stored in the storage unit, and a signal-to-noise ratio corresponding to the current selection or weighting.

6. A pulse wave measuring device that measures a pulse wave at a measurement target site of a living body, the device comprising the antenna device for biological measurement according to claim 5, wherein
the area where the transmission/reception antenna group is provided is placed corresponding to an artery that passes through the measurement target site in the wearing state where the belt is worn as surrounding the outer surface of the measurement target site, and
in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit selects by switching or weights the transmission/reception antenna pair formed of the transmission antenna and the reception antenna among the plurality of antenna elements based on an output from the reception circuit,
further comprising a pulse wave detection unit configured to acquire a pulse wave signal indicating a pulse wave at the artery passing through the measurement target site based on the output from the reception circuit received via the selected or weighted transmission/reception antenna pair.

7. A blood pressure measuring device that measures blood pressure at a measurement target site of a living body, the device comprising two sets of pulse wave measuring devices according to claim 6,
wherein the belts of the two sets are integrally formed,
the transmission/reception antenna group of the two sets are arranged spaced apart from each other in a width direction of the belt,
in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,
in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit selects by switching or weights the transmission/reception antenna pair formed of the transmission antenna and the reception antenna among the plurality of antenna elements based on an output from the reception circuit, and
respectively in the two sets, the pulse wave detection unit acquires the pulse wave signal indicating the pulse wave at the artery passing through the measuring site based on the output from the reception circuit received via the selected or weighted transmission/reception antenna pair,
further comprising:
a time difference acquisition unit configured to acquire a time difference between the pulse wave signals respectively acquired by the pulse wave detection unit of the two sets as a pulse wave transit time; and
a first blood pressure calculation unit configured to calculate blood pressure value based on the pulse wave transit time acquired by the time difference acquisition unit using a predetermined correspondence equation between the pulse wave transit time and the blood pressure.

8. The antenna device for biological measurement according to claim 3, wherein the antenna control unit searches for weighting with which a signal-to-noise ratio of received signal becomes large, by setting an antenna element in the plurality of antenna elements with a relatively heavy weight as sequentially switching from an antenna element arranged at an end in one side to an antenna element arranged at an end on other side in the area where the transmission/reception antenna group is provided in the longitudinal direction of the belt.

9. The antenna device for biological measurement according to claim 3, wherein the antenna control unit searches for weighting with which a signal-to-noise ratio of received signal becomes large, by setting an antenna element in the plurality of antenna elements with a relatively heavy weight as sequentially switching from an antenna element arranged at a central portion to an antenna element arranged at ends in opposite sides alternately in the area where the transmission/reception antenna group is provided in the longitudinal direction of the belt.

10. The antenna device for biological measurement according to claim 3, wherein
the transmission/reception antenna group includes the plurality of antenna elements in M rows and N columns arrangement, where M and N are natural numbers of 2 or more, respectively, and includes the antenna elements arranged to form two transmission antennas along the longitudinal direction of the belt and the antenna elements arranged to form two reception antennas along the longitudinal direction of the belt as two rows and two columns arrangement in the M rows and N columns, and
the antenna control unit searches for weighting with which a signal-to-noise ratio of received signal becomes large, by switching
a first setting that sets a first transmission antenna and a first reception antenna, in the two transmission antennas and the two reception antennas, arranged at one side in the longitudinal direction of the belt with a relatively heavy weight,
a second setting that sets a second transmission antenna and a second reception antenna, in the two transmission antennas and the two reception antennas, arranged at other side in the longitudinal direction of the belt with a relatively heavy weight,
a third setting that sets the first transmission antenna and the second reception antenna with a relatively heavy weight, and
a fourth setting that sets the second transmission antenna and the first reception antenna with a relatively heavy weight.

11. The antenna device for biological measurement according to claim 8, wherein every time the weighting is switched once, the antenna control unit shifts a relative phase of radio waves emitted by the transmission antenna formed by the plurality of antenna elements and/or a relative phase of signals received by the reception antenna formed by the plurality of antenna elements, thereby controlling to increase a signal-to-noise ratio of a combined signal obtained by combining the signals.

12. The antenna device for biological measurement according to claim 8, wherein every time the weighting is switched once, the antenna control unit changes a relative weight of radio waves emitted by the plurality of transmission antennas and a relative weight of signals respectively received by the plurality of reception antennas, thereby controlling to increase a signal-to-noise ratio of a combined signal obtained by combining the signals.

13. A pulse wave measuring device that measures a pulse wave at a measurement target site of a living body, the device comprising the antenna device for biological measurement according to claim 1, wherein
the area where the transmission/reception antenna group is provided is placed corresponding to an artery that passes through the measurement target site in the wearing state where the belt is worn as surrounding the outer surface of the measurement target site, and
in the wearing state, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of antenna element included in the transmission/reception antenna group as the reception antenna, the antenna control unit weights the transmission/reception antenna pair formed of the transmission antenna and the reception antenna among the plurality of antenna elements based on an output from the reception circuit,
further comprising a pulse wave detection unit configured to acquire a pulse wave signal indicating a pulse wave at the artery passing through the measurement target site based on the output from the reception circuit received via the weighted transmission/reception antenna pair.

14. A blood pressure measuring device that measures blood pressure at a measurement target site of a living body, the device comprising two sets of pulse wave measuring devices according to claim 13,
wherein the belts of the two sets are integrally formed,
the transmission/reception antenna group of the two sets are arranged spaced apart from each other in a width direction of the belt,
in the wearing state that the belt is worn as surrounding the outer surface of the measurement target site, an area where a first set of the transmission/reception antenna group of the two sets is provided is placed corresponding to an upstream portion of the artery passing through the measurement target site, while an area where a second set of transmission/reception antenna group is provided is placed corresponding to a downstream portion of the artery,
in the wearing state, respectively in the two sets, while emitting, by the transmission circuit, a radio wave toward the measurement target site using any one of the antenna elements included in the transmission/reception antenna group as the transmission antenna, and receiving, by the reception circuit, a radio wave reflected by the measurement target site using any one of the antenna elements included in the transmission/reception antenna group as the reception antenna, the antenna control unit weights the transmission/reception antenna pair formed of the transmission antenna and the reception antenna among the plurality of antenna elements based on an output from the reception circuit, and
respectively in the two sets, the pulse wave detection unit acquires the pulse wave signal indicating the pulse wave at the artery passing through the measuring site based on the output from the reception circuit received via the weighted transmission/reception antenna pair,
further comprising:
a time difference acquisition unit configured to acquire a time difference between the pulse wave signals respectively acquired by the pulse wave detection unit of the two sets as a pulse wave transit time; and
a first blood pressure calculation unit configured to calculate blood pressure value based on the pulse wave transit time acquired by the time difference acquisition unit using a predetermined correspondence equation between the pulse wave transit time and the blood pressure.

15. The blood pressure measuring device according to claim 7, wherein a fluid bag for pressing the measurement target site is provided to the belt, further comprising:
a pressure control unit configured to control pressure by supplying air in the fluid bag; and
a second blood pressure calculation unit configured to calculate blood pressure by an oscillometric method based on the pressure in the fluid bag.

16. An apparatus comprising the antenna device for biological measurement according to claim 1.

17. A biological information measuring method that measures biological information using a belt to which a transmission/reception antenna group is provided, wherein
the transmission/reception antenna group includes a plurality of antenna elements arranged spaced apart from each other in a longitudinal direction and/or a width direction of the belt,
the biological information measuring method comprising:
wearing the belt as surrounding an outer surface of a measurement target site of the living body into a wearing state so that the transmission/reception antenna group is placed corresponding to an artery passing through the measurement target site; and
in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit.

18. A pulse wave measuring method that measures a pulse wave of a measurement target site of a living body using a belt to which a transmission/reception antenna group is provided, wherein
the transmission/reception antenna group includes a plurality of antenna elements arranged spaced apart from each other in a longitudinal direction and/or a width direction of the belt,
the pulse wave measuring method comprising:
wearing the belt as surrounding an outer surface of a measurement target site into a wearing state so that the transmission/reception antenna group is placed corresponding to an artery passing through the measurement target site;
in the wearing state, while emitting, by a transmission circuit, a radio wave toward the measurement target site using any one of antenna elements included in the transmission/reception antenna group as a transmission antenna and receiving, by a reception circuit, a radio wave reflected by the the measurement target site using any one of antenna elements included in the transmission/reception antenna group as a reception antenna, weighting the transmission/reception antenna pair formed of the transmission antenna and the reception antenna among the plurality of antenna elements based on an output from the reception circuit; and
acquiring a pulse wave signal indicating a pulse wave at the artery passing through the measurement target site based on the output from the reception circuit received via the weighted transmission/reception antenna pair.

19. A blood pressure measuring method that measures blood pressure at a measurement target site of a living body using a belt to which two sets of transmission/reception antenna groups are integrally provided, wherein
the two sets of the transmission/reception antenna groups are arranged spaced apart from each other in a width direction of the belt and respectively include a plurality of antenna elements arranged spaced apart from each other in a longitudinal direction and/or the width direction of the belt,
the blood pressure measuring method comprising:
wearing the belt as surrounding an outer surface of the measurement target site into a wearing state so that a first set of transmission/reception antenna group of the two sets is placed corresponding to an upstream portion of an artery passing through the measurement target site and a second set of transmission/reception antenna group is placed corresponding to a downstream portion of the artery;
in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, weighting a transmission/reception antenna pair formed of the transmission antenna and the reception antenna among the plurality of antenna elements based on an output from the reception circuit;
respectively in the two sets, acquiring a pulse wave signal indicating a pulse wave at the artery passing through the measurement target site based on the output from the reception circuit received via the weighted transmission/reception antenna pair;
acquiring a time difference between the pulse wave signals respectively received in the two sets as a pulse wave transit time; and
calculating a blood pressure value based on the acquired pulse wave transit time using a predetermined correspondence equation between the pulse wave transit time and the blood pressure.

20. A biological information measuring method that measures biological information using a belt to which a transmission/reception antenna group is provided, wherein
the transmission/reception antenna group includes a plurality of antenna elements arranged spaced apart from each other in a longitudinal direction and/or a width direction of the belt,
the biological information measuring method comprising:
wearing the belt as surrounding an outer surface of a measurement target site of a living body into a wearing state so that the transmission/reception antenna group is placed corresponding to an artery passing through the measurement target site;
in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit,
storing a signal-to-noise ratio of received signal corresponding to selection or weighting in a storage unit every time the selection or weighting is switched once, and
determining a next selection or weighting based on a signal-to-noise ratio corresponding to past selection or weighting stored in the storage unit and a signal-to-noise ratio corresponding to current selection or weighting.

21. A pulse wave measuring method that measures a pulse wave at a measurement target site of a living body using a belt to which a transmission/reception antenna group is provided, wherein
the transmission/reception antenna group includes a plurality of antenna elements arranged spaced apart from each other in a longitudinal direction and/or a width direction of the belt,
the pulse wave measuring method comprising:
wearing the belt as surrounding an outer surface of the measurement target site into a wearing state so that the transmission/reception antenna group is placed corresponding to an artery passing through the measurement target site;
in the wearing state, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;
storing a signal-to-noise ratio of received signal corresponding to selection or weighting in a storage unit every time the selection or weighting is switched once;
determing a next selection or weighting based on a signal-to-noise ratio corresponding to past selection or weighting stored in the storage unit and a signal-to-noise ratio corresponding to current selection or weighting; and
acquiring a pulse wave signal indicating a pulse wave at the artery passing through the measurement target site based on the output from the reception circuit received via the selected or weighted transmission/reception antenna pair.

22. A blood pressure measuring method that measures blood pressure at a measurement target site of a living body using a belt to which two sets of transmission/reception antenna groups are integrally provided, wherein
the two sets of the transmission/reception antenna group are arranged spaced apart from each other in a width direction of the belt and respectively include a plurality of antenna elements arranged spaced apart from each other in a longitudinal direction and/or the width direction of the belt,
the blood pressure measuring method comprising:
wearing the belt as surrounding an outer surface of the measurement target site into a wearing state so that a first set of the transmission/reception antenna group of the two sets is placed corresponding to an upstream portion of an artery passing through the measurement target site and a second set of the transmission/reception antenna group is placed corresponding to a downstream portion of the artery;
in the wearing state, respectively in the two sets, while emitting, by a transmission circuit, a radio wave toward the measurement target site using any one of antenna elements included in the transmission/reception antenna group as a transmission antenna and receiving, by a reception circuit, a radio wave reflected by the measurement target site using any one of antenna elements included in the transmission/reception antenna group as a reception antenna, selecting by switching, or weighting a transmission/reception antenna pair formed of the transmission antenna and the reception antenna among the plurality of antenna elements based on an output from the reception circuit;
storing a signal-to-noise ratio of received signal corresponding to selection or weighting in a storage unit every time the selection or weighting is switched once;
determing a next selection or weighting based on a signal-to-noise ratio corresponding to past selection or weighting stored in the storage unit and a signal-to-noise ratio corresponding to current selection or weighting;
respectively in the two sets, acquiring a pulse wave signal indicating a pulse wave at the artery passing through the measurement target site based on the output from the reception circuit received via the selected or weighted transmission/reception antenna pair;
acquiring a time difference between the pulse wave signals respectively acquired in the two sets as a pulse wave transit time; and
calculating a blood pressure value based on the acquired pulse wave transit time using a predetermined correspondence equation between the pulse wave transit time and the blood pressure.
